(19) United States
(12) Patent Application Publication
(10) Pub. No.: US 2007/0042350 A1
(43) Pub. Date: Feb. 22, 2007

(54) Methods and compositions for detecting sars virus and other infectious agents

(76) Inventors: Ze Li, Beijing (CN); Shengee Tao, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 10/564,378

(22) PCT Filed: Jul. 14, 2003
(86) PCT No.: PCT/CN03/00561

§ 371 Date: Jun. 19, 2006

(51) Int. Cl. C12Q 1/70; C12Q 1/68; C12M 3/00

(52) U.S. Cl. 435/5; 435/6; 435/287.2; 977/802; 977/924

(57) ABSTRACT

This invention relates generally to the field of virus detection. In particular, the invention provides chips, probes, primers, kits and methods for amplifying and detecting SARS-CoV nucleotides sequence. The clinical and other uses of the present chips, probes, primers, kits and methods are also contemplated.

### BACKGROUND OF THE INVENTION

Since November of 2002, a disease called severe acute respiratory syndrome (SARS) has been reported in twenty two countries around the world. WHO has reported 6,054 cumulative cases of SARS and 417 death among infected people as of May 2, 2003. For the same period, China has reported 3,788 cumulative cases of SARS and 181 deaths among infected people.

The main symptoms for SARS patients include fever (greater than 38° C.), headache, body aches. After 2-7 days of illness, patients may develop a dry, nonproductive cough that may be accompanied with breathing difficulty.

Based on findings from Hong Kong, Canada, and U.S., a previously unrecognized coronaviruse has been identified as the cause of SARS. Researchers have found that SARS coronaviruse is a positive chain RNA virus which replicates without DNA intermediate step and uses standard codon (Marra et al., Science 2003 May 1; (epub ahead of print); and Rota et al., Science 2003 May 1, (epub ahead of print)).

SARS coronaviruse is a newly discovered virus which has not been previously detected in human or animals. The genome structure of SARS coronaviruse is very similar to other coronaviruse. The genome of SARS coronaviruse is 30 K base pairs in length and the genome is considered very large for a virus. The genome of SARS coronaviruse encodes RNA polymerase (polymerase 1a and 1b), S protein (spike protein), M protein (membrane protein), and N protein (nucleocapsid protein), etc.

Currently, there are three types of detection methods for SARS coronaviruse: immunological methods (e.g., ELISA), reverse transcriptase polymerase chain reaction (RT-PCR) tests, and cell culture methods.

There are significant drawbacks of the above three detection methods. For example, ELISA can reliably detect antibodies from serum of SARS patients. However, those antibodies can only be detected twenty one days after development of symptoms. Cell culture methods have a relative long detection cycle and can be applied only to limited conditions. In addition, cell culture methods can only detect existence of alive virus.

The key step of preventing the spread of SARS coronaviruse is early diagnosis and early quarantine and treatment. RT-PCR is the only existing method that allows detection of nucleic acid of SARS coronaviruse. However, RT-PCR cannot eliminate infected patient before SARS virus expression, and detection rate for RT-PCR is low. The detection process requires expensive real time PCR equipment. Thus, RT-PCR cannot satisfy the need of early clinical screening and diagnosis. There exists a need in the art for a quick, sensitive and accurate diagnosis of the severe acute respiratory syndrome (SARS). The present invention address this and other related needs in the art.

### BRIEF SUMMARY OF THE INVENTION

The current method for clinical diagnosis is mainly based on symptoms such as fever, shadows on patient's lung, dry cough, and weakness in patient's arms and legs. However, these symptoms are not specific for SARS; other pathogens can cause the same or similar symptoms. For example, regular pneumonia caused by Chlamydia pneumoniae and Mycoplasma pneumoniae also generates shadows on patient's lung; fever and cough are also associated with influenza; and similar symptoms are also associated with infection of the upper respiratory tract caused by human coronaviruse 229E and OC43. Thus, diagnosis for SARS solely based on the symptoms of the patient is problematic.

Current clinical data indicate that many suspected SARS cases actually did not have infection by SARS virus, and instead, had infection by other pathogens. Thus, there is a need to develop a method for simultaneous detection of SARS and other pathogens that cause symptoms similarly to SARS. Such method would provide quick screening of suspected cases in order to reduce probability of diagnostic errors, to allow timely and adequate treatment, and to avoid unnecessary panic and medical waste. Patients infected with SARS virus are more susceptible to other pathogens due to decreased immunity caused by SARS virus. It is possible that SARS patients are also infected with other pathogens that generate symptoms similar to SARS. For example, if a patient is infected with both SARS and Mycoplasma pneumoniae, treatment with medicine only for SARS will not make symptoms disappear immediately. In this situation, a simultaneous detection of infection by both pathogens would allow immediate and effective treatment of patients for both pathogens. A biochip-based diagnosis is a fast and low cost method for high throughput simultaneous screening of multiple samples. Thus, one objective of the invention is to provide a biochips for simultaneous detection of SARS virus and other pathogens that cause SARS-like symptoms.

Clinical data also indicate that those SARS patients infected with other pathogens (pathogens that severely interfere and obstruct immunity, such as hepatitis B and HIV) have aggravated symptoms and high probability of infecting others (these patients are called “super-spreaders”). Proper detection of such patients would allow adequate treatment and timely quarantine of patients. Thus, another objective of the invention is to provide a nucleic acid microarray for simultaneous detection of SARS virus and other pathogens that aggravates symptoms of SARS.

In one aspect, the present invention is directed to a chip for assaying for a coronaviruse causing the severe acute respiratory syndrome (SARS-CoV) and a non-SARS-CoV infectious organism, which chip comprises a support suitable for use in nucleic acid hybridization having immobilized thereon an oligonucleotide probe complementary to a nucleotide sequence of SARS-CoV genome, said nucleotide sequence comprising at least 10 nucleotides, and one or more of the following oligonucleotide probe(s): a) an oligonucleotide probe complementary to a nucleotide sequence of a non-SARS-CoV infectious organism causing SARS-like symptoms, said nucleotide sequence comprising at least 10 nucleotides; b) an oligonucleotide probe complementary to a nucleotide sequence of a non-SARS-CoV infectious organism damaging an infectious host's immune system, said nucleotide sequence comprising at least 10 nucleotides; or c) an oligonucleotide probe complementary to a nucleotide sequence of a non-SARS-CoV coronaviridae virus, said nucleotide sequence comprising at least 10 nucleotides.

In some embodiments, the chip of the invention comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

In some embodiments, the non-SARS-CoV infectious organism causing SARS-like symptoms is selected from the group consisting of a human coronaviruse 229E, a human coronaviruse OC43, a human enteric coronaviruse, an influenza virus, a parainfluenza virus, a respiratory sncytical virus, a human metapneumovirus, a rhinovirus, an adenoviruse, a mycoplasma pneumoniae, a chlamydia pneumoniae, a measles virus and a rubella virus.

In some embodiments, the non-SARS-CoV infectious organism damaging an infectious host's immune system is selected from the group consisting of a hepatitis virus, a transfusion transmitting virus (TTV), a human immunodeficiency virus (HI), a parvovirus, a human cytomegalovirus (HCMV), an Epstein-Barr virus (EBV) and a tre-ponema palidum.

In another aspect, the present invention is directed to a method for assaying for a SARS-CoV and a non-SARS-CoV infectious organism in a sample, which methods comprises: a) providing an above-described chip; b) contacting said chip with a sample containing or suspected of containing a nucleotide sequence of a SARS-CoV and a non-SARS-CoV infectious organism under conditions suitable for nucleic acid hybridization; and c) assessing hybrids formed between said nucleotide sequence of said SARS-CoV or said non-SARS-CoV infectious organism, if present in said sample, and said oligonucleotide probe complementary to a nucleotide sequence of said SARS-CoV genome or said oligonucleotide probe complementary to a nucleotide sequence of said non-SARS-CoV infectious organism genome, whereby detection of one or both of said hybrids indicates the presence of said SARS-CoV and/or said non-SARS-CoV infectious organism in said sample.

In some embodiments, the SARS-CoV is assayed by: a) providing a chip comprising a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotide; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample, whereby detection of one or both said hybrids indicates the presence of said SARS-CoV in said sample.

By using multiple hybridization probes, the present methods reduce the occurrence of false negative results compared to a test based on a single hybridization probe as the chance of simultaneous mutations of the multiple hybridization targets is much smaller than the chance of a mutation in the single hybridization target. When other preferred embodiments are used, e.g., a negative control probe and a blank spot on the chip, the chance of a false positive result can also be reduced. The inclusion of more preferred embodiments, e.g., an immobilization control probe and a positive control probe, on the chip can provide further validation of the assay results. The use of preferred sample preparation procedures, RNA extraction procedures and amplification procedures can further enhance the sensitivity of the present methods.

In still another aspect, the present invention is directed to an oligonucleotide primer for amplifying a nucleotide sequence of an influenza A virus, an influenza B virus, a human metapneumovirus, a human adenovirus, a human coronaviruse 229E or a human coronaviruse OC43, which oligonucleotide primer comprises a nucleotide sequence that: a) hybridizes, under high stringency, with a target nucleotide sequence of influenza A virus, influenza B virus, human metapneumovirus, human adenovirus, human coronaviruse 229E or human coronaviruse OC43, or a complementary strand thereof, that is set forth in Tables 1-6; or b) has at least 90% identity to a target nucleotide sequence of influenza A virus, influenza B virus, human metapneumovirus, human adenovirus, human coronaviruse 229E or human coronaviruse OC43 comprising a nucleotide sequence, or a complementary strand thereof, that is set forth in Tables 1-6.

TABLE 1
Exemplary Influenza A Virus Primers

| | |
|---|---|
| Id | Sequence |

| | |
|---|---|
| PMIA_00001 | TTTGTGCGACAATGCTTCA |
| PMIa_00002 | GACATTTGAGAAAGCTTGCC |
| PMia_00003 | AGGGACAACCTNGAACCTGG |
| PMIA_00004 | AGGAGTTGMCCAAGACGCATT |
| PMIA_00005 | ACCACATTCCCTTATACTGGAG |
| PMIA_00006 | TTAGTCATCATCTTTCTCACAACA |
| PMIA_00007 | ACAAATTGCTTCMATGAGAAC |
| PMIA_00008 | TGTCTCCGAAGAAATAAGATCC |
| PMIA_00009 | GCGCAGAGACTTGAAGATGT |
| PMIA_00010 | CCTTCCGTAGAAGGCCCT |

TABLE 2
Exemplary Influenza B Virus Primers

| | |
|---|---|
| Id | Sequence |

| | |
|---|---|
| PMIB_00001 | CACAATGGCAGAATTTAGTGA |
| PMIB_00002 | GTCAGTTTGATCCCGTAGTG |
| PMIB_00003 | CAGATCCCAGAGTGGACTCA |
| PMIB_00004 | TGTATTACCCAAGGGTTGTTAC |
| PMIB_00005 | GATCAGCATGACAGTAACAGGA |
| PMIB_00006 | ATGTTCGGTAAAAGTCGTTTAT |
| PMIB_00007 | CCACAGGGGAGATTCCAAAG |
| PMIB_00008 | GACATTCTTCCTGATTCATAATC |
| PMIB_00009 | CAAACAACGGTAGACCAATATA |
| PMIB_00010 | AGGTTCAGTATCTATCACAGTCTT |
| PMIB_00011 | ATGTCCAACATGGATATTGAC |
| PMIB_00012 | GCTCTTCCTATAAATCGAATG |
| PMIB_00013 | TGATCAAGTGATCGGAAGTAG |
| PMIB_00014 | GATGGTCTGCTTAATTGGAA |
| PMIB_00015 | ACAGAAGATGGAGAAGGCAA |
| PMIB_00016 | ATTGTTTCTTTGGCCTGGAT |

TABLE 3
Exemplary Human Metapneumovirus Primers

| | |
|---|---|
| Id | Sequence |

| | |
|---|---|
| PMM_00001 | CATCCCAAAAATTGCCAGAT |
| PMM_00002 | TTTGGGCT1TGCCTTAAATG |
| PMM_00003 | ACACCCTCATCATTGCAACA |
| PMM_00004 | GCCCTTCTGACTGTGGTCTC |
| PMM_00005 | CGACACAGCAGCAGGAATTA |
| PMM_00006 | TCAAAGCTGCTTGACACTGG |
| PMM_00007 | CAAGTGCGACATTGATGACC |
| PMM_00008 | TAATTCCTGCTGCTGTGTCG |
| PMM_00009 | GCGACTGTAGCACTTGACGA |
| PMM_000010 | TCATGATCAGTCCCGCATAA |
| PMM_000011 | TGTTTCAGGCCAATACACCA |
| PMM_000012 | TCATGATCAGTCCCGCATAA |
| PMM_000013 | TCATGGGTAATGAAGCAGCA |
| PMM_000014 | GGAGTTTTCCCATCACTGGA |
| PMM_000015 | TCCAGTGATGGGAAAACTCC |
| PMM_000016 | TGTTGAGCTCCTTTGCCTTT |

TABLE 4
Exemplary Human Adenovirus Primers

| | |
|---|---|
| Id | Sequence |

| | |
|---|---|
| PMAd1_00001 | TGGCGGTATAGGGGTAACTG |
| PMAd1_00002 | ATTGCGGTGATGGTTAAAGG |
| PMAd1_00003 | TTTTGCCGATCCCACTTATC |
| PMAd1_00004 | GCAAGTCTACCACGGCATTT |
| PMAd2_00001 | CTCCGTTATCGCTCCATGTT |
| PMAd2_00002 | AAGGACTGGTCGTTGGTGTC |
| PMAd2_00003 | AAATGCCGTGGTAGATTTGC |
| PMAd2_00004 | GTTGAAGGGGTTGACGTTGT |
| PMAd3_00001 | TCCTCTGGATGGCATAGGAC |
| PMAd3_00002 | TGTTGGTGTTAGTGGGCAAA |
| PMAd3_00003 | ACATGGTCCTGCAAAGTTCC |
| PMAd3_00004 | GCATTGTGCCACGTTGTATC |
| PMAd4_00001 | CGCTTCGGAGTACCTCAGTC |
| PMAd4_00002 | CTGCATCATTGGTGTCAACC |
| PMAd4_00003 | GGCAGCTTTTACCTCAACCA |
| PMAd4_00004 | TCTGGACCAAGAACCAGTCC |
| PMAd5_00001 | GGCCTACCCTGCTAACTTCC |
| PMAd5_00002 | ATAAAGAAGGGTGGGCTCGT |
| PMAd5_00003 | ATCGCAGTTGAATGCTGTTG |
| PMAd5_00004 | GTTGAAGGGGTTGACGTTGT |
| PMAd7_00001 | ACATGGTCCTGCAAAGTTCC |
| PMAd7_00002 | GATCGAACCCTGATCCAAGA |
| PMAd7_00003 | AACACCAACCGAAGGAGATG |
| PMAd7_00004 | CCTATGCCATCCAGAGGAAA |
| PMAd11_00001 | CAGATGCTCGCCAACTACAA |
| PMAd11_00002 | AGCCATGTAACCCACAAAGO |
| PMAd11_00003 | ACGGACGTTATGTGCCTTTC |
| PMAd11_00004 | GGGAATATTGGTTGCATTGG |
| PMAd21_00001 | ACTGGTTCCTGGTCCAGATG |
| PMAd21_00002 | AGCCATGTAACCCACAAAGC |
| PMAd21_00003 | CTGGATATGGCCAGCACTTT |
| PMAd21_00004 | CACCTGAGGTTCTGGTTGGT |
| PMAd23_00001 | TAATGAAAAGGGCGGACAAG |
| PMAd23_90002 | GGCAATGTAGTTTGGCCTGT |
| PMAd23_00003 | AACTCCGCGGTAGACAGCTA |
| PMAd23_00004 | CGTAGGTGTTGGTGTTGGTG |

TABLE 5
Exemplary HCoV-OC229E Primers

| | |
|---|---|
| Id | Sequence |

| | |
|---|---|
| PMV_a0053 | TCACTTGCTTCCGTTGAGGTTGGGCTGGCGGTTTAGAGTTG |
| | A |
| PMV_a0054 | GGTTTCGGATGTTACAGCGTGTGCGACCGCCCTTGTTTATG |
| | G |
| PMV_a0055 | TCACTTGCTTCCGTTGAGGGCGTTGTTGGCCTTTTTCTTGT |
| | CT |
| PMV_a0056 | GGTTTCGGATGTTACAGCGTGCCCGGCATTATTTCATTGTT |
| | CTG |
| PMV_a0057 | TCACTTGCTTCCGTTGAGGACAAAAGCCGCTGGTGGTAAAG |
| PMV_a0058 | GGTTTCGGATGTTACAGCGTCAGAAATCATAACGGGCAAAC |
| | TCA |
| PMV_a0059 | TCACTTGCTTCCGTTGAGGAAGAGTTATTGCTGGCGTTGTT |
| | GG |
| PMV_a0060 | GGTTTCGGATGTTACAGCGTGCCCGGCATTATTTCATTGTT |
| | CTG |
| PMV_b0053 | TTGGGCTGGCGGTTTAGAGTTGA |
| PMV_b0054 | GTGCGACCGCCCTTGTTTATGG |
| PMV_b0055 | GCGTTGTTGGCCTTTTTCTTGTCT |
| PMV_b0056 | GCCCGGCATTATTTCATTGTTCTG |
| PMV_b0057 | ACAAAAGCCGCTGGTGGTAAAG |
| PMV_b0058 | CAGAAATCATAACGGGCAAACTCA |
| PMV_b0059 | AAGAGTTATTGCTGGCGTTGTTGG |
| PMV_b0060 | GCCCGGCATTATTTCATTGTTCTG |

TABLE 6
Exemplary HCoV-OC43 Primers

| | |
|---|---|
| Id | Sequence |

| | |
|---|---|
| PMV_a0061 | TCACTTGCTTCCGTTGAGGTTGGGGTGATGGGTTTCAGATT |
| | AA |
| PMV_a0062 | GGTTTCGGATGTTACAGCGTCTCGGGGAAGATCGCCTTCTT |
| | CTA |
| PMV_b0061 | TTGGGGTGATGGGTTTCAGATTAA |
| PMV_b0062 | CTCGGGAAGATCGCCTTCTTCTA |

In yet another aspect, the present invention is directed to a kit for amplifying a nucleotide sequence of an influenza A virus, an influenza B virus, a human metapneumovirus, a human adenovirus, a human coronaviruse 229E or a human coronaviruse OC43, which kit comprises: a) a primer described above; and b) a nucleic acid polymerase that can amplify a nucleotide sequence of an influenza A virus, an influenza B virus, a human metapneumovirus, a human adenovirus, a human coronaviruse 229E or a human coronaviruse OC43 using said primer.

In yet another aspect, the present invention is directed to an oligonucleotide probe for hybridizing to a nucleotide sequence of an influenza A virus, an influenza B virus, a human metapneumovirus, a human adenovirus, a human coronaviruse 229E or a human coronaviruse OC43, which oligonucleotide probe comprises a nucleotide sequence that: a) hybridizes, under high stringency, with a target nucleotide sequence of influenza A virus, influenza B virus, human metapneumovirus, human adenovirus, human coronaviruse 229E or human coronaviruse OC43, or a complementary strand thereof, that is set forth in Tables 7-12; or b) has at least 90% identity to a target nucleotide sequence of influenza A virus, influenza B virus, human metapneumovirus, human adenovirus, human coronaviruse 229E or human coronaviruse OC43, or a complementary strand thereof, that is set forth in Tables 7-12.

TABLE 7
Exemplary Influenza A Virus Probes

| | |
|---|---|
| Id | Sequence |

| | |
|---|---|
| PBIA_00001 | TTTAGAGCCTATGTGGATGGATTCRAACCGAACGGCTGC |
| | ATTGAGGGCAAGCTTTCTCAAATGTC |
| PBIA_00002 | ACAATTGAAGAAAGATTTGAAATCACTGGAACCATGCGC |
| | AGGCTTGCCGACCAAAGTCTCCCACCGAACT |
| PBIA_00003 | AGCAATNGAGGAGTGCCTGATTAANGATCCCTGGGTTTT |
| | GCTNAATGC |
| PBIA_00004 | CCATACAGCCATGGAACAGGAACAGGATACACCATGGAC |
| | ACAGTCAACAGAACACANCAATATTCAGAAA |
| PBIA_00005 | GGGCGGGGAGTCTTCGAGCTCTCNGACGAAAAGGCAACG |
| | AACCCGATCGTGCC |
| PBIA_00006 | GATCTNGAGGCTCTCATGGAATGGCTAAAGACAAGACCA |
| | ATCCTGTCACCTCTGACTAA |

TABLE 8
Exemplary Influenza B Virus Probes

| | |
|---|---|
| Id | Sequence |

| | |
|---|---|
| PBIB_00001 | GCTGGGAAATAGCATGGAACTGATGATATTCAGCTACAA |
| | TCAAGACTATTCGTTAAGTAATGAATCCTCA |
| PBIB_00002 | TCTGTTCCAGCTGGTTTCTCCAATTTTGAAGGAATGAGG |
| | AGCTACATAGACAATATAGATCCTAAAGGAG |
| PBIB_00003 | TTACAACCATGAGCTACCAGAAGTTCCATATAATGCCTT |
| | TCTTCTAATGTCTGATGAATTGGGGCTGGCC |
| PBIB_00004 | ACAAATAAGATCCAAATGAAATGGGGAATGGAAGCTAGA |
| | AGATGTCTGCTTCAATCAATGCAACAAATGG |
| PBIB_00005 | GAGGGAATGTATTCTGGAATAGANGAATGTATTAGTAAC |
| | AACCCTTGGGTAATACAGAGTGCATACTGGT |
| PBIB_00006 | CTACCGTGTTGGGAGTAGCCGCACTAGGTATCAAAAACA |
| | TTGGAAACAAAGAATACTTATGGGATGGACT |
| PBIB_00007 | GGCTATGACTGAAAGAATAACCAGAGACAGCCCAATTTG |
| | GTTCCGGGATTTTTGTAGTATAGCACCGGTC |
| PBIB_00008 | ACTGATCAGAGGAACATGATTCTTGAGGAACAATGCTAC |
| | GCTAAGTGTTGCAACCTTTTTGAGGCCTGTT |
| PBIB_00009 | AAAATCCCTTTGTNGGACATTTGTCTATTGAGGGCATCA |
| | AAGANGCAGATATAACCCCAGCACATGGTCC |
| PBIB_00010 | CTTGGAATACAAGGGAATACAACTTAAAACAAATGCTGA |
| | AGACATAGGAACCAAAGGCCAAATGTGCTCA |
| PBIB_00011 | GTGGCAGGAGCAACATCAGCTGAGTTCATAGAAATGCTA |
| | CACTGCTTACAAGGTGAAATTGGAGACAAA |
| PBIB_00012 | GGAACCCATCCCCGGAAAGAGCAACCACAAGCAGTGAAG |
| | CTGATGTCGGAAGGAAAACCCAAAAGAAACA |
| PBIB_00013 | CTGTTTCCAAAGATCAAAGGCACTAAAAAGAGTTGGACT |
| | TGACCCTTCATTAATCAGTACCTTTGCAGGA |
| PBIB_00014 | AGAGTTTTGTCTGCATTAACAGGCACAGAATTCAAGCCT |
| | AGATCAGCATTAAAATGCAAGGGTTTCCATG |
| PBIB_00015 | GAGGGACGTGATGCAGATGTCAAAGGAAATCTACTCAAG |
| | ATGATGAATGACTCAATGGCTAAGAAAACCA |
| PBIB_00016 | CCTATCAGGAATGGGAACAACAGCAACAAAAAAGAAAGG |
| | CCTGATTCTAGCTGAGAGAAAAATGAGAAGA |
| PBIB_00017 | GGAAGTCAAAAGAATGGGGAAGGAATTGCAAAGGATGTA |
| | ATGGAAGTGCTAAAGCAGAGCTCTATGGGAA |

TABLE 9
Exemplary Human Metapneumovirus Probes

| | |
|---|---|
| Id | Sequence |

| | |
|---|---|
| PBM_00001 | AAAAGTGTATCACAGAAGTTTGTTCATTGAGTATGGCAAAG |
| | CATTAGGCTCATCATCTACAGGCAGCAAA |
| PBM_00002 | GAAAGTCTATTTGTTATATATATTCATGCAAGCTTATGGAG |
| | CCGGTCAAACAATGCTAAGGTGGGGGGTCA |
| PBM_00003 | ACGCTGTTGTGTGGAGAAATTGTGTATGCTAAACATGCTGA |
| | TTACAAATATGCTGCAGAAATAGGAATAC |
| PBM_00004 | TTAAGGAATCATCAGGTAATATCCCACAAAATCAGAGGCCC |
| | TCAGCACCAGACACACCCATAATCTTATT |
| PBM_00005 | TGAGCAATCAAAGGAGTGCAACATCAACATATCCACTACAA |
| | ATTACCCATGCAAAGTCAGCACAGGAAGA |
| PBM_00006 | CTGTTCCATTGGCAGCAACAGAGTAGGGATCATCAAGCAGC |
| | TGAACAAAGGTTGCTCCTATATAACCAAC |
| PBM_00007 | ACTTAATGACAGATGCTGAACTAGCCAGGGCCGTTTCTAAC |
| | ATGCCGACATCTGCAGGACAAATAAAATT |
| PBM_00008 | AAAAAAAGGGAAACTATGCTTGCCTCTTAAGAGAAGACCAA |
| | GGGTGGTATTGTGAGAATGCAGGGTCAAC |
| PBM_00009 | GAAAAGAACACACCAGTTACAATACCAGCATTTATCAAATC |
| | GGTTTCTATCAAAGAGAGTGAATCAGCCA |
| PBM_00010 | CAAATCAGTTGGCAAAAAAACACATGATCTGATCGCATTAT |
| | GTGATTTTATGGATCTAGAAAAGAACACA |
| PBM_00011 | CAGCTAAAGACACTGACTATAACTACTCTGTATGCTGCATC |
| | ACAAAGTGGTCCAATACTAAAAGTGAKTG |
| PBM_00012 | AAAAGAACACACCAGTTACAATACCAGCATTTATCAAATCG |
| | GTTTCTATCAAAGAGAGTGAATCAGCCAC |
| PBM_00013 | CTATTATAGGAGAAAAAGTGAACACTGTATCTGAAACATTG |
| | GAATTACCTACTATCAGTAGACCCACCAA |
| PBM_00014 | AAGTTAGCATGGACAGACAAAGGTGGGGCAATCAAAACTGA |
| | AGCAAAGCAAACAATCAAAGTTATGGATC |
| PBM_00015 | CAGGAAAATACACAAAGTTGGAGAAAGATGCTCTAGACTTG |
| | CTTTCAGACAATGAAGAAGAAGATGCAGA |
| PBM_00016 | CTAATAGCAGACATAATAAAAGAAGCCAAGGGAAAAGCAGC |
| | AGAAATGATGGAAGAAGAAATGAACCAGC |

TABLE 10
Exemplary Human Adenovirus Probes

| | |
|---|---|
| Id | Sequence |

| | |
|---|---|
| PBAd_00001 | CTGACACCTACCAAGGTATAAAATCAAACGGAAACGGTA |
| | ATCCTCAAAACTGGACCAAAAATGACGATTT |
| PBAd_00002 | TCCTCTACTCCAACATTGCACTGTACCTGCCTGACAAGC |
| | TAAAATACACTCCTACAAATGTGGAAATATC |
| PBAd_00003 | GCTATCGGAGGCAGAGTACTAAAAAAGACTACTCCCATG |
| | AAACCATGCTACGGATCGTATGCCAGACCTA |
| PBAd_00004 | AGTATTGTTTTGTACAGTGAGGATGTTAATATGGAAACT |
| | CCTGATACTCACATTTCATACAAACCAAGCA |
| PBAd_00005 | GGGAAACGATCTTAGAGTTGACGGGGCTAGCATTAAGTT |
| | TGACAGCATTTGTCTTTACGCCACCTTCTTC |
| PBAd_00006 | TTGCCATTAAAAACCTCCTCCTCCTGCCAGGCTCATATA |
| | CATATGAATGGAACTTCAGGAAGGATGTTAA |
| PBAd_00007 | TTGCAACACGTAATGAAATAGGAGTGGGTAACAACTTTG |
| | CCATGGAAATTAACCTAAATGCCAACCTATG |
| PBAd_00008 | TTGGGGTAACTGACACCTATCAAGCTATTAAGGCTAATG |
| | GCAATGGCTCAGGCGATAATGGAGATATTAC |
| PBAd_00009 | AGGTATCAAGGCATTAAAGTTAAAACCGATGACGCTAAT |
| | GGATGGGAAAAATGCTAATGTTGATACAG |
| PBAd_00010 | GAGAAGTTTTCTGTACTCCAATGTGGCTTTGTACCTTCC |
| | AGATGTTTACAAGTACACGCCACCTAACATT |
| PBAd_00011 | ATCAGTCATTTAACGACTACCTCTCTGCAGCTAACATGC |
| | TTTACCCCATTCCTGCCAATGCAACCAACAT |
| PBAd_00012 | CTACTTCGTATATTCTGGATCTATTCCCTACCTGGATGG |
| | CACCTTTTACCTTAACCACACTTTCAAGAAG |
| PBAd_00013 | ACCTGCCAGTGGAAGGATGCTAACAGCAAAATGCATACC |
| | TTTGGGGTAGCTGCCATGCCAGGTGTTACTG |
| PBAd_00014 | ATAGAAGCTGATGGGCTGCCTATTAGAATAGATTCAACT |
| | TCTGGAACTGACACAGTAATTTATGCTGATA |
| PBAd_00015 | TTGAAATTAAGCGCACCGTGGACGGCGAGGGGTACAACG |
| | TGGCCCAGTGCAACATGACCAAGGACTGGTT |
| PBAd_00016 | CGGCAACGACCGGCTCCTGACGCCCAACGAGTTTGAAAT |
| | TAAGCGCACCGTGGACGGCGAGGGGTACAAC |
| PBAd_00017 | CTCCAGTAACTTTATGTCCATGGGCGCACTCACAGACCT |
| | GGGCCAAAACCTTCTCTACGCCAACTCCGCC |
| PBAd_00018 | GCTAACTTCCCCTATCCGCTTATAGGCAAGACCGCAGTT |
| | GACAGCATTACCCAGAAAAAGTTTCTTTGCG |
| PBAd_00019 | ACAGTCCTTCCAACGTAAAAATTTCTGATAACCCAAACA |
| | CCTACGACTACATGAACAAGCGAGTGGTGGC |
| PBAd_00020 | AAGATGAACTTCCAAATTACTGCTTTCCACTGGGAGGTG |
| | TGATTAATACAGAGACTCTTACCAAGGTAAA |
| PBAd_00021 | AGCTAACATGCTTTACCCCATCCCTGCCAATGCAACCAA |
| | CATTCCAATTTCCATCCCATCTCGCAACTGG |
| PBAd_00022 | TTCAACTCTTGAAGCCATGCTGCGCAACGATACCAATGA |
| | TCAGTCATTCAACGACTACCTCTCTGCAGCT |
| PBAd_00023 | AGGCTGTGGACAGCTATGATCCCGATGTTCGTATTATTG |
| | AAAATCATGGCGTCGAGGATGAACTGCCTAA |
| PBAd_00024 | TGAAATTGTGCTTTACACGGAAAATGTCAATTTGGAAAC |
| | TCCAGACAGCCATGTGGTATACAAGCCAGGA |
| PBAd_00025 | CATCGGCTATCAGGGCTTCTACATTCCAGAAGGATACAA |
| | AGATCGCATGTATTCATTTTTCAGAAACTTC |
| PBAd_00026 | GCTGCTTCTCCCAGGCTCCTACACTTATGAGTGGAACTT |
| | TAGGAAGGATGTGAACATGGTTCTACAGAGT |
| PBAd_00027 | ATGACACCAATGATCAGTCATTCAACGACTACCTATCTGC |
| | AGCTAACATGCTCTACCCCATTCCTGCCAA |
| PBAd_00028 | CTTGCCAACTACAACATTGGATACCAGGGCTTCTACGTT |
| | CCTGAGGGTTACAAGGATCGCATGTACTCCT |
| PBAd_00029 | GATCGCATGTACTCCTTCTTCAGAAACTTCCAGCCCATG |
| | AGTAGACAGGTGGTTGATGAGATTAACTACA |
| PBAd_00030 | CCCCTAAGGGCGCTCCCAATACATCTCAGTGGATTGCTG |
| | AAGGCGTAAAAAAAGAAGATGGGGGATCTGA |
| PBAd_00031 | AGAAAATGTAAATTTGGAAACTCCAGATTCCCATGTTGT |
| | TTACAAAGCAGGAACTTCAGACGAAAGCTCT |
| PBAd_00032 | TGTGGCTACCAATACTGTTTACCAAGGTGTTAAGTTACA |
| | AACTGGTCAAACTGACAAATGGCAGAAAGAT |
| PBAd_00033 | CCFGAATTGGGAAGGGTAGCGTATTCGCCATGGAAATCA |
| | ATCTCCAGGCCAACCTGTGGAAGAGTTTTCTG |
| PBAd_00034 | TTGATGAGGTCAATTACAAAGACTTCAAGGCCGTCGCCA |
| | TACCCTACCAACACAACAACTCTGGCTTTGT |
| PBAd_00035 | TGACGAAGAGGAAGAGAAAAATCTCACCACTTACACTTT |
| | TGGAAATGCCCCAGTGAAAGCAGAAGGTGGT |
| PBAd_00036 | AGAAGATTTTGACATTGACATGGCTTTCTTTGATTCCAA |
| | CACTATTAACACACCAGATGTTGTGCTGTAT |

TABLE 11
Exemplary HCoV-OC229E Probes

| | |
|---|---|
| Id | Sequence |

| | |
|---|---|
| PBS10049 | AATGGGGTTATGTTGGTTCACTCTCCACTAATCACCATGCAA |
| | TTTGTAATGTTCATAGAAATGAGCATGT |
| PBS10050 | GTGTATGACTGCTTTGTTAAGAATGTGGATTGGTCAATTACC |
| | TACCCTATGATAGCTAATGAAAATGCCA |
| PBS10051 | TTGCATCTTCTTTTGTTGGTATGCCATCTTTTGTTGCATATG |
| | AAACAGCAAGACAAGAGTATGAAAATGC |
| PBS10052 | AAATGGTTCCTCACCACAAATAATCAAACAATTGAAGAAGGC |
| | TATGAATGTTGCAAAAGCTGAGTTTGAC |
| PBS10053 | CTGCTGCAGCTATGTACAAAGAAGCACGTGCTGTTAATAGAA |
| | AATCAAAAGTTGTTAGTGCCATGCATAG |
| PBS10054 | ACGTTTGGACATGTCTAGTGTTGACACTATCCTTAATATGGC |
| | ACGTAATGGTGTTGTCCCTCTTTCCGTT |
| PBS10055 | CTGGTGGTAAAGTTTCATTTTCTGATGACGTTGAAGTAAAAG |
| | ACATTGAACCTGTTTACAGAGTCAAGCT |
| PBS10056 | TTTACAGAGTCAAGCTTTGCTTTGAGTTTGAAGATGAAAAAC |
| | TTGTAGATGTTTGTGAAAAGGCAATTGG |
| PBS10057 | GATGTTTGTGAAAAGGCAATTGGCAAGAAAATTAAACATGAA |
| | GGTGACTGGGATAGCTTTTGTAAGACTA |
| PBS10058 | GCGTTGTTGGCCTTTTTCTTGTCTAAGCATAGTGATTTTGGT |
| | CTTGGTGATCTTGTCGATTCTTATTTTG |
| PBS10059 | AGCAAGACAAGAGTATGAAAATGCTGTTGCAAATGGTTCCTC |
| | ACCACAAATAATCAAACAATTGAAGAAG |
| PBS10060 | TTGAAGAAGGCTATGAATGTTGCAAAAGCTGAGTTTGACAGG |
| | GAATCATCTGTTCAAAAGAAAATTAACA |
| PBS10061 | CTGCTGCAGCTATGTACAAAGAAGCACGTGCTGTTAATAGAA |
| | AATCAAAAGTTGTTAGTGCCATGCATAG |

TABLE 12
Exemplary HCoV-OC43 Probes

| | |
|---|---|
| Id | Sequence |

| | |
|---|---|
| PBS10062 | CTCACATCCTAGGAAGATGCATAGTTTTAGATGTTAAAGGTG |
| | TAGAAGAATTGCATGACGATTTAGTTAA |
| PBS10063 | GGATTGGCCATTGCACCATAGCTCAACTCACGGATGCAGCAC |
| | TGTCCATTAAGGAAAATGTTGATTTTAT |
| PBS10064 | GCATGCAATTCAATTATAAAATCACCATCAACCCCTCATCAC |
| | CGGCTAGACTTGAAATAGTTAAGCTCGG |
| PBS10065 | ATAGTTAGTCACTGGATGGGAATTCGTTTTGAATACACATCA |
| | CCCACTGATAAGCTAGCTATGATTATGG |

In yet another aspect, the present invention is directed to a kit for hybridization analysis of a nucleotide sequence of an influenza A virus, an influenza B virus, a human metapneumovirus, a human adenovirus, a human coronaviruse 229E or a human coronaviruse OC43, which kit comprises: a) a above-described probe; and b) a means for assessing a hybrid formed between a nucleotide sequence of an influenza A virus, an influenza B virus, a human metapneumovirus, a human adenovirus, a human coronaviruse 229E or a human coronaviruse OC43 and said probe.

### BRIEF DESCRIPTION OF THE SEVERAL VIEWS OF THE DRAWING(S)

FIGS. 1A and 1B illustrate exemplary SARS-CoV genome structures (See Figure 2 of Marra et al., Science 2003 May 1; [epub ahead of print]; and GenBank Accession No. NC_{—}004718).

FIG. 2 illustrates an exemplary sample preparation procedure.

FIG. 3 illustrates an exemplary probe labeling to be used in PCR. The sequence of the universal primer is complementary to the common sequence of the specific primer. The universal primers and the specific primers are added into the PCR master mix before the amplification are performed. The specificity of the amplification is ensured by the specific part of the specific primer. After one or a few thermal cycles, the universal primer can be incorporated into the amplicon efficiently. Then the universal primer can anneal to the complementary sequence of the common sequence of the specific primer The PCR can further proceed with the fluorescence dye incorporated in the universal primer. 1 and 6 depict a fluorescence dye; 2 depicts an upstream universal primer; 3 depicts an upstream specific primer with a common sequence; 4 depicts a template; 5 depicts a downstream specific primer with a common sequence; and 7 depicts a downstream universal primer.

FIG. 4 illustrates probe immobilization on a glass slide surface modified with an amino group, e.g., poly-L-lysine treated. Amine Coupling Chemistry: Amine Substrates contain primary amine groups (NH3+) attached covalently to the glass surface (rectangles). The amines carry a positive charge at neutral pH, allowing attachment of natively charged DNA (double helix) through the formation of ionic bonds with the negatively charged phosphate backbone (middle panel). Electrostatic attachment is supplemented by treatment with an ultraviolet light or heat, which induces covalent attachment of the DNA to the surface through the covalent binding between the primary amine and thymine (right panel). The combination of electrostatic binding and covalent attachment couples the DNA to the substrate in a highly stable manner.

FIG. 5 illustrates an exemplary array format of SARS-CoV detection chip.

FIGS. 6A and 6B illustrate SARS-CoV detection from a SARS patient blood sample (sample No. 3).

FIGS. 7A and 7B illustrate SARS-CoV detection from a SARS patient blood sample (sample No. 4).

FIGS. 8A and 8B illustrate SARS-CoV detection from a SARS patient sputum sample (sample No. 5).

FIGS. 9A and 9B illustrate SARS-CoV detection from a SARS patient sputum sample (sample No. 6).

FIG. 10 illustrates another exemplary array format of SARS-CoV detection chip.

FIG. 11 illustrates all possible positive results on the SARS SARS-CoV detection chip illustrated in FIG. 10.

FIG. 12 illustrates another exemplary array format of SARS-CoV detection chip.

FIG. 13 illustrates all possible positive results on the SARS SARS-CoV detection chip illustrated in FIG. 12.

FIG. 14 illustrates all possible positive and negative results on the SARS SARS-CoV detection chip illustrated in FIG. 12.

### DETAILED DESCRIPTION OF THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “coronaviridae” refers to a family of single-stranded RNA viruses responsible for respiratory diseases. The outer envelope of the virus has club-shaped projections that radiate outwards and give a characteristic corona appearance to negatively stained virions.

As used herein, “polymerase chain reaction (PCR)” refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and a heat-stable DNA polymerase, e.g., Taq DNA polymerase. In a series, e.g., 30, of temperature cycles, the target DNA is repeatedly denatured (e.g., around 90° C.), annealed to the primers (e.g., at 50-60° C.) and a daughter strand extended from the primers (e.g., 72° C.). As the daughter strands themselves act as templates for subsequent cycles, DNA fragments matching both primers are amplified exponentially, rather than linearly. The original DNA need thus be neither pure nor abundant, and the PCR reaction has accordingly become widely used not only in research, but in clinical diagnostics and forensic science.

As used herein, “nested PCR” refers to a PCR in which specificity is improved by using two sets of primers sequentially. An initial PCR is performed with the “outer” primer pairs, then a small aliquot is used as a template for a second round of PCR with the “inner” primer pair.

As used herein, “reverse transcription PCR or RT-PCR” refers to PCR in which the starting template is RNA, implying the need for an initial reverse transcriptase step to make a DNA template. Some thermostable polymerases have appreciable reverse transciptase activity; however, it is more common to perform an explicit reverse transcription, inactivate the reverse transcriptase or purify the product, and proceed to a separate conventional PCR.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection. The term “target sequence” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “the concentration of said 5′ and 3′ universal primers equals to or is higher than the concentration of said 5′ and 3′ specific primers, respectively” means that the concentration of the 5′ universal primer equals to or is higher than the concentration of the 5′ specific primers and the concentration of the 3′ universal primer equals to or is higher than the concentration of the 3′ specific primers.

As used herein, “hairpin structure” refers to a polynucleotide or nucleic acid that contains a double-stranded stem segment and a single-stranded loop segment wherein the two polynucleotide or nucleic acid strands that form the double-stranded stem segment is linked and separated by the single polynucleotide or nucleic acid strand that forms the loop segment. The “hairpin structure” can further comprise 3′ and/or 5′ single-stranded region(s) extending from the double-stranded stem segment.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “complementary or matched” means that two nucleic acid sequences have at least 50% sequence identity. Preferably, the two nucleic acid sequences have at least 60%, 70,%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows:

1) high stringency: 0.1×SSPE (or 0.1×SSC), 0.1% SDS, 65° C.;

2) medium stringency: 0.2×SSPE (or 1.0×SSC), 0.1% SDS, 50° C. (also referred to as moderate stringency); and

3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “melting temperature” (“Tm”) refers to the midpoint of the temperature range over which nucleic acid duplex, i.e., DNA:DNA, DNA:RNA, RNA:RNA, PNA:DNA, LNA:RNA and LNA:DNA, etc., is denatured.

As used herein, “sample” refers to anything which may contain a target SARS-CoV to be assayed or amplified by the present chips, primers, probes, kits and methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “assessing PCR products” refers to quantitative and/or qualitative determination of the PCR products, and also of obtaining an index, ratio, percentage, visual or other value indicative of the level of the PCR products. Assessment may be direct or indirect and the chemical species actually detected need not of course be the PCR products themselves but may, for example, be a derivative thereof, or some further substance.

B. Chips for Assaying for a SARS-CoV and a Non-SARS-CoV Infectious Organism

In one aspect, the present invention is directed to a chip for assaying for a coronaviruse causing the severe acute respiratory syndrome (SARS-CoV) and a non-SARS-CoV infectious organism, which chip comprises a support suitable for use in nucleic acid hybridization having immobilized thereon an oligonucleotide probe complementary to a nucleotide sequence of SARS-CoV genome, said nucleotide sequence comprising at least 10 nucleotides, and one or more of the following oligonucleotide probe(s): a) an oligonucleotide probe complementary to a nucleotide sequence of a non-SARS-CoV infectious organism causing SARS-like symptoms, said nucleotide sequence comprising at least 10 nucleotides; b) an oligonucleotide probe complementary to a nucleotide sequence of a non-SARS-CoV infectious organism damaging an infectious host's immune system, said nucleotide sequence comprising at least 10 nucleotides; or c) an oligonucleotide probe complementary to a nucleotide sequence of a non-SARS-CoV coronaviridae virus, said nucleotide sequence comprising at least 10 nucleotides.

In some embodiments, the chip comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

The at least two different nucleotide sequences can be any suitable combinations. For example, the at least two different nucleotide sequences of SARS-CoV genome can comprise a nucleotide sequence of at least 10 nucleotides located within a conserved region of SARS-CoV genome and a nucleotide sequence of at least 10 nucleotides located within a variable region of SARS-CoV genome. In another example, the at least two different nucleotide sequences of SARS-CoV genome can comprise a nucleotide sequence of at least 10 nucleotides located within a structural protein coding gene of SARS-CoV genome and a nucleotide sequence of at least 10 nucleotides located within a non-structural protein coding gene of SARS-CoV genome.

If desired, the present chips can comprise other types of probes or other features. For example, the chip can further comprise: a) at least one of the following three oligonucleotide probes: an immobilization control probe that is labeled and does not participate in any hybridization reaction when a sample containing or suspected of containing of a SARS-CoV or a non-SARS-CoV infectious organism is contacted with the chip, a positive control probe that is not complementary to any SARS-CoV or non-SARS-CoV infectious organism sequence but is complementary to a sequence contained in the sample not found in the SARS-CoV or the non-SARS-CoV infectious organism and a negative control probe that is not complementary to any nucleotide sequence contained in the sample; and b) a blank spot.

In a specific embodiment, the present chips can comprise at least two oligonucleotide probes complementary to two different nucleotide sequences of at least 10 nucleotides, respectively, located within a conserved region of SARS-CoV genome, located within a structural protein coding gene of SARS-CoV genome or located within a non-structural protein coding gene of SARS-CoV genome.

Any conserved region of SARS-CoV genome can be used as assay target. For example, the conserved region of SARS-CoV genome can be a region located within the Replicase 1A, 1B gene or the Nucleocapsid (N) gene of SARS-CoV.

Any variable region of SARS-CoV genome can be used as assay target. For example, the variable region of SARS-CoV genome can be a region located within the Spike glycoprotein (S) gene of SARS-CoV.

Any structural protein coding gene of SARS-CoV genome can be used as assay target. For example, the structural protein coding gene of SARS-CoV genome can be a gene encoding the Spike glycoprotein (S), the small envelope protein (E) or the Nucleocapsid protein (N).

Any non-structural protein coding gene of SARS-CoV genome can be used as assay target. For example, the non-structural protein coding gene of SARS-CoV genome can be a gene encoding the Replicase 1A or 1B.

In another specific embodiment, the present chips can comprise at least two of the following four oligonucleotide probes: two oligonucleotide probes complementary to two different nucleotide sequences of at least 10 nucleotides located within the Replicase 1A or 1B gene of SARS-CoV, an oligonucleotide probe complementary to a nucleotide sequence of at least 10 nucleotides located within the N gene of SARS-CoV and an oligonucleotide probe complementary to a nucleotide sequence of at least 10 nucleotides located within the S gene of SARS-CoV.

Preferably, one or both of the different nucleotide sequences located within the Replicase 1A or 1B gene of SARS-CoV can comprise a nucleotide sequence that: a) hybridizes, under high stringency, with a Replicase 1A or 1B nucleotide sequence, or a complementary strand thereof, that is set forth in Table 13; or b) has at least 90% identity to a Replicase 1A or 1B nucleotide sequence comprising a nucleotide sequence, or a complementary strand thereof, that is set forth in Table 13. More preferably, one or both of the different nucleotide sequences located within the Replicase 1A or 1B gene of SARS-CoV comprises a nucleotide sequence that is set forth in Table 13.

TABLE 13
Exemplary SARS-CoV probes

| | | |
|---|---|---|
| probe_id | Sequence 5′-3′ | region |

| | | |
|---|---|---|
| PBS00001 | TTACCCTAATATGTTTATCACCCGCGAAGAAGCTATTCGTCACGTTCGTGCGTGGA | SARS-CoV Replicase 1B |
| PBS00002 | CTGACAAGTATGTCCGCAATGTACAACACAGGCTCTATGAGTGTCTCTATAGAAAT | SARS-CoV Replicase 1B |
| PBS00003 | CATAACACTTGCTGTAACTTATCACACCGTTTCTACAGGTTAGCTAACGAGTGTGC | SARS-CoV Replicase 1B |
| PBS00004 | TTACCCTAATATGTTTATCACCCGCGAAGAAGCTATTCGTCACGTTCGTG | SARS-CoV Replicase 1B |
| PBS00009 | GCGTTCTCTTAAAGCTCCTGCCGTAGTGTCAGTATCATCACCAGATGCTGTTACTACATATAATGGATAC | SARS-CoV Replicase 1A |
| PBS00010 | CTTTGGCTGGCTCTTACAGAGATTGGTCCTATTCAGGACAGCGTACAGAGTTAGGTGTTGAATTTCTTAA | SARS-CoV Replicase 1A |
| PBS00011 | CTACGTAGTGAAGCTTTCGAGTACTACCATACTCTTGATGAGAGTTTTCTTGGTAGGTACATGTCTGCTT | SARS-CoV Replicase 1A |
| PBS00012 | TGCCAATTGGTTATGTGACACATGGTTTTAATCTTGAAGAGGCTGCGCCCTGTATGCGTTCTCTTAAAGC | SARS-CoV Replicase 1A |
| PBS00013 | TATAAAGTTACCAAGGGAAAGCCCGTAAAAGGTGCTTGGAACATTGGACAACAGAGATCAGTTTTAACAC | SARS-CoV Replicase 1A |
| PBS00014 | TGCTTCATTGATGTTGTTAACAAGGCACTCGAAATGTGCATTGATCAAGTCACTATCGCTGGCGCAAAG | SARS-CoV Replicase 1A |
| PBS00015 | TGTCGACGCCATGGTTTATACTTCAGACCTGCTCACCAACAGTGTCATTATTATGGCATATGTAACTGGT | SARS-CoV Replicase 1A |
| PBS00016 | TACTGTTGAAAAACTCAGGCCTATCTTTGAATGGATTGAGGCGAAACTTAGTGCACCAGTTGAATTTCTC | SARS-CoV Replicase 1A |
| PBS00017 | ACCTATTCTGTTGCTTGACCAAGCTCTTGTATCAGACGTTGGAGATAGTACTGAAGTTTCC | SARS-CoV Replicase 1A |
| PBS00018 | GCCTATTAATGTCATAGTTTTTGATGGCAAGTCCAAATGCGACGAGTCTGCTTCTAAGTCTGCTTCTGTG | SARS-CoV Replicase 1A |
| PBS00019 | TGAGAGCTAACAACACTAAAGGTTCACTGCCTATTAATGTCATAGTTTTTGATGGCAAGTCCAAATGCGA | SARS-CoV Replicase 1A |
| PBS00020 | ACTTGCATGATGTGCTATAAGCGCAATCGTGCCACACGCGTTGAGTGTACAACTATTGTTAATGGCATGA | SARS-CoV Replicase 1A |
| PBS00021 | GGCGATGTAGTGGCTATTGACTATAGACACTATTCAGCGAGTTTCAAGAAAGGTGCTAAATTACTGCATA | SARS-CoV Replicase 1A |
| PBS00022 | TCAAACCAAACACTTGGTGTTTACGTTGTCTTTGGAGTACAAAGCCAGTAGATACTTCAAATTCATTTGA | SARS-CoV Replicase 1A |
| PBS00023 | TAGTGCTGTTGGCAACATTTGCTACACACCTTCCAAACTCATTGAGTATAGTGATTTTGCTAC | SARS-CoV Replicase 1A |
| PBS00024 | TCATAGCTAACATCTTTACTCCTCTTGTGCAACCTGTGGGTGCTTTAGATGTCTCTGCTTCAGTAGTGG | SARS-CoV Replicase 1A |
| PBS00025 | GGTATTATTGCCATATTGGTGACTTGTGCTGCCTACTACTTTATGAAATTCAGACGTCTTTTTGGTCAGT | SARS-CoV Replicase 1A |
| PBS00026 | GTGATGTCAGAGAAACTATGACCCATCTTCTACAGCATGCTAATTTGGAATCTGCAAAGCGAGTTCTTAA | SARS-CoV Replicase 1A |
| PBS00027 | AACCATCAAGCCTGTGTCGTATAAACTCGATGGAGTTACTTACACAGAGATTGAACCAAAATTGGATGGG | SARS-CoV Replicase 1A |
| PBS00028 | GTTTTCTACAAGGAAACATCTTACACTACAACCATCAAGCCTGTGTCCTATAAACTCGATGGAGTTACTT | SARS-CoV Replicase 1A |
| PBS00029 | CCTTGAATGAGGATCTCCTTGAGATACTGAGTCGTGAACCTGTTAACATTAACATTGTTGGCGATTTTCA | SARS-CoV Replicase 1A |
| PBS00031 | GCCATGGTTTATACTTCAGACCTGCTCACCAACAGTGTCATTATTATGGCATATGTAACTGGTGGTCTTG | SARS-CoV Replicase 1A |
| PBS00032 | CAACAGACTTCTCAGTGGTTGTCTAATCTTTTGGGCACTACTGTTGAAAAACTCAGGCCTATCTTTGAAT | SARS-CoV Replicase 1A |
| PBS00033 | TTCCCGTCAGGCAAAGTTGAAGGGTGCATGGTACAAGTAACCTGTGGAACTACAAC | SARS-CoV Replicase 1A |
| PBS00034 | GGTTCACCATCTGGTGTTTATCAGTGTGCCATGAGACCTAATCATACCATTAAAGG | SARS-CoV Replicase 1A |
| PBS00035 | AGATCATGTTGACATATTGGGACCTCTTTCTGCTCAAACAGGAATTGCCGTC | SARS-CoV Replicase 1A |
| PBS00036 | TAAAAAGGACAAAAAGAAAAAGACTGATGAAGCTCAGCCTTTGCCCGCAGAGACAAAAGAAGCAGCCCACT | SARS-CoV Nucleocapsid |
| | | gene |
| PBS00037 | ACGGCAAAATGAAAGAGCTCAGCCCCAGATGGTACTTCTATTACCTAGGAACTGGCCCAGAAGCTTCACT | SARS-CoV Nucleocapsid |
| | | gene |
| PBS00038 | GGCGCTAACAAAGAAGGCATCGTATGGGTTGCAACTGAGGGAGCCTTGAATACACCCAAAGACCACATTG | SARS-CoV Nucleocapsid |
| | | gene |
| PBS00039 | GTCCAGATGACCAAATTGGCTACTACCGAAGAGCTACCCGACGAGTTCGTGGTGGTGACGGCAAAAATGAA | SARS-CoV Nucleocapsid |
| | | gene |
| PBS00040 | GAGGTGGTGAAACTGCCCTCGCGCTATTGCTGCTAGACAGATTGAACCAGCTTGAGAGCAAAGTTTCTGG | SARS-CoV Nucleocapsid |
| | | gene |
| PBS00041 | AAAAAGAAAAAGACTGATGAAGCTCAGCCTTTGCCGCAGAGACAAAAGAAGCAGCCCACTGTGACTCTTCT | SARS-CoV Nucleocapsid |
| | | gene |
| PBS00042 | AAATTGCACAATTTGCTCCAAGTGCCTCTGCATTCTTTGGAATGTCACGCATTGGCATGGAAGTCACACC | SARS-CoV Nucleocapsid |
| | | gene |
| PBS00043 | ACCAATTTAACAAGGCGATTAGTCAAATTCAAGAATCACTTACAACAACATCAACTGCATTGGGCAAGCT | SARS-Cov Spike glyco- |
| | | protein gene |
| PBS00044 | CACCTGGAACAAATGCTTCATCTGAAGTTGCTGTTCTATATCAAGATGTTAACTGCACTGATGTTTCTAC | SARS-Cov Spike glyco- |
| | | protein gene |
| PBS00045 | AAAGGGCTACCACCTTATGTCCTTCCCACAAGCACCCCGCATGGTGTTGTCTTCCTACATGTCACGTAT | SARS-Cov Spike glyco- |
| | | protein gene |
| PBS00046 | TCAGGAAATTGTGATGTCGTTATTGGCATCATTAACAACACAGTTTATGATCCTCTGCAACCTGAGCTTG | SARS-Cov Spike glyco- |
| | | protein gene |
| PBS00047 | TTGATCTTGGCGACATTTCAGGCATTAACGCTTCTGTCGTCAACATTCAAAAAGAAATTGACCGCCTCAA | SARS-Cov Spike glyco- |
| | | protein gene |
| PBS00048 | GAGGAACTTCACCACAGCGCCAGCAATTTGTCATGAAGGCAAAGCATACTTCCCTCGTGAAGGTGTTTTT | SARS-Cov Spike glyco- |
| | | protein gene |

Also preferably, the nucleotide sequence located within the N gene of SARS-CoV can comprise a nucleotide sequence that: a) hybridizes, under high stringency, with a N nucleotide sequence, or a complementary strand thereof, that is set forth in Table 13; or b) has at least 90% identity to a N nucleotide sequence comprising a nucleotide sequence, or a complementary strand thereof, that is set forth in Table 13. More preferably, the nucleotide sequence located within the N gene of SARS-CoV comprises a nucleotide sequence that is set forth in Table 13.

Also preferably, the nucleotide sequence located within the S gene of SARS-CoV can comprise a nucleotide sequence that: a) hybridizes, under high stringency, with a S nucleotide sequence, or a complementary strand thereof, that is set forth in Table 13; or b) has at least 90% identity to a S nucleotide sequence comprising a nucleotide sequence, or a complementary strand thereof, that is set forth in Table 13. More preferably, the nucleotide sequence located within the S gene of SARS-CoV comprises a nucleotide sequence that is set forth in Table 13.

Any suitable label can be used in the immobilization control probe, e.g., a chemical, an enzymatic, an immunogenic, a radioactive, a fluorescent, a luminescent or a FRET label.

Any suitable non-SARS-CoV-sequence can be used. For example, the non-SARS-CoV-sequence can be an endogenous component of a sample to be assayed. Alternatively, the non-SARS-CoV-sequence is spiked in the sample to be assayed. In another example, the spiked non-SARS-CoV-sequence can be a sequence of Arabidopsis origin.

In still another specific embodiment, the present chips can comprise two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe that is labeled and does not participate in any hybridization reaction when a sample containing or suspected of containing of a SARS-CoV or a non-SARS-CoV infectious organism is contacted with the chip, a positive control probe that is not complementary to any SARS-CoV sequence but is complementary to any sequence contained in the sample not found in the SARS-CoV or the non-SARS-CoV infectious organism and a negative control probe that is not complementary to any nucleotide sequence contained in the sample.

Preferably, the chip comprises multiple spots of the described probes, e.g., multiple spots of the two oligonucleotide probes complementary to two different nucleotide sequences located within the Replicase 1A or 1B gene of SARS-CoV, the oligonucleotide probe complementary to a nucleotide sequence located within the N gene of SARS-CoV, the oligonucleotide probe complementary to a nucleotide sequence located within the S gene of SARS-CoV, the immobilization control probe, the positive control probe and the negative control probe.

The present chips can further comprise an oligonucleotide probe complementary to a nucleotide sequence of a coronaviruse not related to the SARS-CoV. For example, the coronaviruse not related to the SARS can be the Group I, II or III coronaviruse or is a coronaviruse that infects an avian species, e.g., Avian infectious bronchitis virus and Avian infectious laryngotracheitis virus, an equine species, e.g., Equine coronaviruse, a canine species, e.g., Canine coronaviruse, a feline species, e.g., Feline coronaviruse and Feline infectious peritonitis virus, a porcine species, e.g., Porcine epidemic diarrhea virus, Porcine transmissible gastroenteritis virus and Porcine hemagglutinating encephalomyelitis virus, a calf species, e.g., Neonatal calf diarrhea coronaviruse, a bovine species, e.g., Bovine coronaviruse, a murine species, e.g., Murine hepatitis virus, a puffinosis species, e.g., Puffinosis virus, a rat species, e.g., Rat coronaviruse and a Sialodacryoadenitis virus of rat, e.g., a turkey species e.g., Turkey coronaviruse, or a human species, e.g., Human enteric coronaviruse. The present chips can further comprise an oligonucleotide probe complementary to a nucleotide sequence of other types of virus or pathogens. An exemplary list of viruses and pathogens that can be assayed using the present chips is set forth in the following Table 14.

TABLE 14
Exemplary viruses and pathogens

| | | | Sample | |
|---|---|---|---|---|
| | | | nucleic | |
| No. | Virus name | Genome | acid | Structure |
| 1 | Coronaviridae | Single-stranded, | RNA | Having capsid |
| | | linear RNA | | |
| 2 | SARS-CoV | Single-stranded, | RNA | Having capsid |
| | | linear RNA | | |
| 3 | Human | Single-stranded, | RNA | Having capsid |
| | coronaviruse | linear RNA | | |
| | 229E | | | |
| 4 | Human | Single-stranded, | RNA | Having capsid |
| | coronaviruse | linear RNA | | |
| | OC43 | | | |
| 5 | Influenzavirus | Single-stranded, | RNA | Having capsid |
| | A, B, C | linear RNA, | | |
| | | fragmented | | |
| 6 | Parainfluenza | Single-stranded, | RNA | Having capsid |
| | virus | linear RNA | | |
| 7 | Respiratory | Single-stranded, | RNA | Having capsid |
| | sncytical virus | linear RNA | | |
| 8 | Human | Single-stranded, | RNA | Having capsid |
| | metapneumovirus | linear RNA | | |
| 9 | Rhinovirus | Single-stranded | RNA | No capsid |
| | | RNA | | |
| 10 | Adenoviruse | Double-stranded, | DNA | No capsid |
| | | linear DNA | | |
| 11 | Mycoplasma | Double-stranded, | DNA and | Having cell |
| | pneumoniae | linear DNA | RNA | wall |
| 12 | Chlamydia | Double-stranded, | DNA and | No cell wall |
| | pneumoniae | linear DNA | RNA | |

The various probes, e.g., the oligonucleotide probe complementary to a nucleotide sequence located within a conserved region of SARS-CoV genome, the oligonucleotide probe complementary to a nucleotide sequence located within a variable region of SARS-CoV genome, the immobilization control probe, the positive control probe or the negative control probe the oligonucleotide probe complementary to a nucleotide sequence of a non-SARS-CoV infectious organism causing SARS-like symptoms, the oligonucleotide probe complementary to a nucleotide sequence of a non-SARS-CoV infectious organism damaging an infectious host's immune system, and the oligonucleotide probe complementary to a nucleotide sequence of a non-SARS-CoV coronaviridae virus, can comprise, at its '5 end, a poly dT region to enhance its immobilization on the support.

In a specific embodiment, the at least one of the oligonucleotide probes is complementary to a highly expressed nucleotide sequence of SARS-CoV genome. Such a chip is particularly useful in detecting early-stage SARS-CoV infection.

In some embodiments, the non-SARS-CoV infectious organism is an infectious organism causing SARS-like symptoms. Such organism includes, but not limited to, a human coronaviruse 229E, a human coronaviruse OC43, a human enteric coronaviruse, an influenza virus, a parainfluenza virus, a respiratory sncytical virus, a human metapneumovirus, a rhinovirus, an adenoviruse, a mycoplasma pneumoniae, a chlamydia pneumoniae, a measles virus and a rubella virus. The influenza virus can be influenza virus A or influenza virus B. The parainfluenza virus can be parainfluenza virus 1, parainfluenza virus 2, parainfluenza virus 3, or parainfluenza virus 4. Exemplary probes for these organisms are set forth in Table 15.

TABLE 15

Exemplary probes for non-SARS-CoV infectious
organisms causing SARS-like symptoms

| | | |
|---|---|---|
| seqid | sequence (5′-3′) | species |

| | | |
|---|---|---|
| PBIA_00001 | TTTAGAGCCTATGTGGATGGA | Influenza A virus |
| | TTCRAACCGAACGGCTGCATT | |
| | GAGGGCAAGCTTTCTCAAATG | |
| | TC | |
| PBIA_00002 | ACAATTGAAGAAAGATTTGAA | Influenza A virus |
| | ATCACTGGAACCATGCGCAGG | |
| | CTTGCCGACCAAAGTCTCCCA | |
| | CCGAACT | |
| PBIA_00003 | AGCAATNGAGGAGTGCCTGAT | Influenza A virus |
| | TAANGATCCCTGGGTTTTGCT | |
| | NAATGC | |
| PBIA_00004 | CCATACAGCCATGGAACAGGA | Influenza A virus |
| | ACAGGATACACCATGGACACA | |
| | GTCAACAGAACACANCAATAT | |
| | TCAGAAA | |
| PBIA_00005 | GGGCGGGGAGTCTTCGAGCTC | Influenza A virus |
| | TCNGACGAAAAGGCAACGAAC | |
| | CCGATCGTGCC | |
| PBIA_00006 | GATCTNGAGGCTCTCATGGAA | Influenza A virus |
| | TGGCTAAAGACAAGACCAATC | |
| | CTGTCACCTCTGACTAA | |
| PBIB_00001 | GCTGGGAAATAGCATGGAACT | Influenza B virus |
| | GATGATATTCAGCTACAATCA | |
| | AGACTATTCGTTAAGTAATGA | |
| | ATCCTCA | |
| PBIB_00002 | TCTGTTCCAGCTGGTTTCTCC | Influenza B virus |
| | AATTTTGAAGGAATGAGGAGC | |
| | TACATAGACAATATAGATCCT | |
| | AAAGGAG | |
| PBIB_00003 | TTACAACCATGAGCTACCAGA | Influenza B virus |
| | AGTTCCATATAATGCCTTTCT | |
| | TCTAATGTCTGATGAATTGGG | |
| | GCTGGCC | |
| PBIB_00004 | ACAAATAAGATCCAAATGAAA | Influenza B virus |
| | TGGGGAATGGAAGCTAGAAGA | |
| | TGTCTGCTTCAATCAATGCAA | |
| | CAAATGG | |
| PBIB_00005 | GAGGGAATGTATTCTGGAATA | Influenza B virus |
| | GANGAATGTATTAGTAACAAC | |
| | CCTTGGGTAATACAGAGTGCA | |
| | TACTGGT | |
| PBIB_00006 | CTACCGTGTTGGGAGTAGCCG | Influenza B virus |
| | CACTAGGTATCAAAAACATTG | |
| | GAAACAAAGAATACTTATGGG | |
| | ATGGACT | |
| PBIB_00007 | GGCTATGACTGAAAGAATAAC | Influenza B virus |
| | CAGAGACAGCCCAATTTGGTT | |
| | CCGGGATTTTTGTAGTATAGC | |
| | ACCGGTC | |
| PBIB_00008 | ACTGATCAGAGGAACATGATT | Influenza B virus |
| | CTTGAGGAACAATGCTACGCT | |
| | AAGTGTTGCAACCTTTTTGAG | |
| | GCCTGTT | |
| PBIB_00009 | AAAATCCCTTTGTNGGACATT | Influenza B virus |
| | TGTCTATTGAGGGCATCAAAG | |
| | ANGCAGATATAACCCCAGCAC | |
| | ATGGTCC | |
| PBIB_00010 | CTTGGAATACAAGGGAATACA | Influenza B virus |
| | ACTTAAAACAAATGCTGAAGA | |
| | CATAGGAACCAAAGGCCAAAT | |
| | GTGCTCA | |
| PBIB_00011 | GTGGCAGGAGCAACATCAGCT | Influenza B virus |
| | GAGTTCATAGAAATGDCTACA | |
| | CTGCTTACAAGGTGAAAATTG | |
| | GAGACAAA | |
| PBIB_00012 | GGAACCCATCCCCGGAAAGAG | Influenza B virus |
| | CAACCACAAGCAGTGAAGCTG | |
| | ATGTCGGAAGGAAAACCCAAA | |
| | AGAAACA | |
| PBIB_00013 | CTGTTTCCAAAGATCAAAGGC | Influenza B virus |
| | ACTAAAAAGAGTTGGACTTGA | |
| | CCCTTCATTAATCAGTACCTT | |
| | TGCAGGA | |
| PBIB_00014 | AGAGTTTTGTCTGCATTAACA | Influenza B virus |
| | GGCACAGAATTCAAGCCTAGA | |
| | TCAGCATTAAAATGCAAGGGT | |
| | TTCCATG | |
| PBIB_00015 | GAGGGACGTGATGCAGATGTC | Influenza B virus |
| | AAAGGAAATCTACTCAAGATG | |
| | ATGAATGACTCAATGGCTAAG | |
| | AAAACCA | |
| PBIB_00016 | CCTATCAGGAATGGGAACAAC | Influenza B virus |
| | AGCAACAAAAAAGAAAGGCCT | |
| | GATTCTAGCTGAGAGAAAAAT | |
| | GAGAAGA | |
| PBIB_00017 | GCAAGTCAAAAGAATGGGGAA | Influenza B virus |
| | GGAATTGCAAAGGATGTAATG | |
| | GAAGTGCTAAAGCAGAGCTCT | |
| | ATGGGAA | |
| PBAd_00001 | CTGACACCTACCAAGGTATAA | Human adenovirus |
| | AATCAAACGGAAACGGTAATC | |
| | CTCAAAACTGGACCAAAAATG | |
| | ACGATTT | |
| PBAd_00002 | TCCTCTACTCCAACATTGCAC | Human adenovirus |
| | TGTACCTGCCTGACAAGCTAA | |
| | AATACACTCCTACAAATGTGG | |
| | AAATATC | |
| PBAd_00003 | GCTATCGGAGGCAGAGTACTA | Human adenovirus |
| | AAAAAGACTACTCCCATGAAA | |
| | CCATGCTACGGATCGTATGCC | |
| | AGACCTA | |
| PBAd_00004 | AGTATTGTTTTGTACAGTGAG | Human adenovirus |
| | GATGTTAATATGGAAACTCCT | |
| | GATACTCACATTTCATACAAA | |
| | CCAAGCA | |
| PBAd_00005 | GGGAAACGATCTTAGAGTTGA | Human adenovirus |
| | CGGGGCTAGCATTAAGTTTGA | |
| | CAGCATTTGTCTTTACGCCAC | |
| | CTTCTTC | |
| PBAd_00006 | TTGCCATTAAAAACCTCCTCC | Human adenovirus |
| | TCCTGCCAGGCTCATATACAT | |
| | ATGAATGGAACTTCAGGAAGG | |
| | ATGTTAA | |
| PBAd_00007 | TTGCAACACGTAATGAAATAG | Human adenovirus |
| | GAGTGGGTAACAACTTTGCCA | |
| | TGGAAATTAACCTAAATGCCA | |
| | ACCTATG | |
| PBAd_00008 | TTGGGGTAACTGACACCTATC | Human adenovirus |
| | AAGCTATTAAGGCTAATGGCA | |
| | ATGGCTCAGGCGATAATGGAG | |
| | ATATTAC | |
| PBAd_00009 | AGGTATCAAGGCATTAAAGTT | Human adenovirus |
| | AAAACCGATGACGCTAATGGA | |
| | TGGGAAAAAGATGCTAATGTT | |
| | GATACAG | |
| PBAd_00010 | GAGAAGTTTTCTGTACTCCAA | Human adenovirus |
| | TGTGGCTTTGTACCTTCCAGA | |
| | TGTTTACAAGTACACGCCACC | |
| | TAACATT | |
| PBAd_00011 | ATCAGTCATTTAACGACTACC | Human adenovirus |
| | TCTCTGCAGCTAACATGCTTT | |
| | ACCCCATTCCTGCCAATGCAA | |
| | CCAACAT | |
| PBAd_00012 | CTACTTCGTATATTCTGGATC | Human adenovirus |
| | TATTCCCTACCTGGATGGCAC | |
| | CTTTTACCTTAACCACACTTT | |
| | CAAGAAG | |
| PBAd_00013 | ACCTGCCAGTGGAAGGATGCT | Human adenovirus |
| | AACAGCAAAATGCATACCTTT | |
| | GGGGTAGCTGCCATGCCAGGT | |
| | GTTACTG | |
| PBAd_00014 | ATAGAAGCTGATGGGCTGCCT | Human adenovirus |
| | ATTAGAATAGATTCAACTTCT | |
| | GGAACTGACACAGTAATTTAT | |
| | GCTGATA | |
| PBAd_00015 | TTGAAATTAAGCGCACCGTGG | Human adenovirus |
| | ACGGCGAGGGGTACAACGTGG | |
| | CCCAGTGCAACATGACCAAGG | |
| | ACTGGTT | |
| PBAd_00016 | CGGCAACGACCGGCTCCTGAC | Human adenovirus |
| | GCCCAACGAGTTTGAAATTAA | |
| | GCGCACCGTGGACGGCGAGGG | |
| | GTACAAC | |
| PBAd_00017 | CTCCAGTAACTTTATGTCCAT | Human adenovirus |
| | GGGCGCACTCACAGACCTGGG | |
| | CCAAAACCTTCTCTACGCCAA | |
| | CTCCGCC | |
| PBAd_00018 | GCTAACTTCCCCTATCCGCTT | Human adenovirus |
| | ATAGGCAAGACCGCAGTTGAC | |
| | AGCATTACCCAGAAAAAGTTT | |
| | CTTTGCG | |
| PBAd_00019 | ACAGTCCTTCCAACGTAAAAA | Human adenovirus |
| | TTTCTGATAACCCAAACACCT | |
| | ACGACTACATGAACAAGCGAG | |
| | TGGTGGC | |
| PBAd_00020 | AAGATGAACTTCCAAATTACT | Human adenovirus |
| | GCTTTCCACTGGGAGGTGTGA | |
| | TTAATACAGAGACTCTTACCA | |
| | AGGTAAA | |
| PBAd_00021 | AGCTAACATGCTTTACCCCAT | Human adenovirus |
| | CCCTGCCAATGCAACCAACAT | |
| | TCCAATTTCCATCCCATCTCG | |
| | CAACTGG | |
| PBAd_00022 | TTCAACTCTTGAAGCCATGCT | Human adenovirus |
| | GCGCAACGATACCAATGATCA | |
| | GTCATTCAACGACTACCTCTC | |
| | TGCAGCT | |
| PBAd_00023 | AGGCTGTGGACAGCTATGATC | Human adenovirus |
| | CCGATGTTCGTATTATTGAAA | |
| | ATCATGGCGTCGAGGATGAAC | |
| | TGCCTAA | |
| PBAd_00024 | TGAAATTGTGCTTTACACGGA | Human adenovirus |
| | AAATGTCAATTTGGAAACTCC | |
| | AGACAGCCATGTGGTATACAA | |
| | GCCAGGA | |
| PBAd_00025 | CATCGGCTATCAGGGCTTCTA | Human adenovirus |
| | CATTCCAGAAGGATACAAAGA | |
| | TCGCATGTATTCATTTTTCAG | |
| | AAACTTC | |
| PBAd_00026 | GCTGCTTCTCCCAGGCTCCTA | Human adenovirus |
| | CACTTATGAGTGGAACTTTAG | |
| | GAAGGATGTGAACATGGTTCT | |
| | ACAGAGT | |
| PBAd_00027 | ATGACACCAATGATCAGTCAT | Human adenovirus |
| | TCAACGACTACCTATCTGCAG | |
| | CTAACATGCTCTACCCCATTC | |
| | CTGCCAA | |
| PBAd_00028 | CTTGCCAACTACAACATTGGA | Human adenovirus |
| | TACCAGGGCTTCTACGTTCCT | |
| | GAGGGTTACAAGGATCGCATG | |
| | TACTCCT | |
| PBAd_00029 | GATCGCATGTACTCCTTCTTC | Human adenovirus |
| | AGAAACTTCCAGCCCATGAGT | |
| | AGACAGGTGGTTGATGAGATT | |
| | AACTACA | |
| PBAd_00030 | CCCCTAAGGGCGCTCCCAATA | Human adenovirus |
| | CATCTCAGTGGATTGCTGAAG | |
| | GCGTAAAAAAAGAAGATGGGG | |
| | GATCTGA | |
| PBAd_00031 | AGAAAATGTAAATTTGGAAAC | Human adenovirus |
| | TCCAGATTCCCATGTTGTTTA | |
| | CAAAGCAGGAACTTCAGACGA | |
| | AAGCTCT | |
| PBAd_00032 | TGTGGCTACCAATACTGTTTA | Human adenovirus |
| | CCAAGGTGTTAAGTTACAAAC | |
| | TGGTCAAACTGACAAATGGCA | |
| | GAAAGAT | |
| PBAd_00033 | CCGAATTGGGAAGGGTAGCGT | Human adenovirus |
| | ATTCGCCATGGAAATCAATCT | |
| | CCAGGCCAACCTGTGGAAGAG | |
| | TTTTCTG | |
| PBAd_00034 | TTGATGAGGTCAATTACAAAG | Human adenovirus |
| | ACTTCAAGGCCGTCGCCATAC | |
| | CCTACCAACACAACAACTCTG | |
| | GCTTTGT | |
| PBAd_00035 | TGACGAAGAGGAAGAGAAAAA | Human adenovirus |
| | TCTCACCACTTACACTTTTGG | |
| | AAATGCCCCAGTGAAAGCAGA | |
| | AGGTGGT | |
| PBAd_00036 | AGAAGATTTTGACATTGACAT | Human adenovirus |
| | GGCTTTCTTTGATTCCAACAC | |
| | TATTAACACACCAGATGTTGT | |
| | GCTGTAT | |
| PBS10062 | CTCACATCCTAGGAAGATGCA | HCoV-OC43 |
| | TAGTTTTAGATGTTAAAGGTG | |
| | TAGAAGAATTGCATGACGATT | |
| | TTAGTTAA | |
| PBS10063 | GGATTGGCCATTGCACCATAG | HCoV-OC43 |
| | CTCAACTCACGGATGCAGCAC | |
| | TGTCCATTAAGGAAAATGTTG | |
| | GATTTTAT | |
| PBS10064 | GCATGCAATTCAATTATAAAA | HCoV-OC43 |
| | TCACCATCAACCCCTCATCAC | |
| | CGGCTAGACTTGAAATAGTTA | |
| | AAGCTCGG | |
| PBS10065 | ATAGTTAGTCACTGGATGGGA | HCoV-OC43 |
| | ATTCGTTTTGAATACACATCA | |
| | CCCACTGATAAGCTAGCTATG | |
| | ATTATGG | |
| PBS10049 | AATGGGGTTATGTTGGTTCAC | HCoV-229E |
| | TCTCCACTAATCACCATGCAA | |
| | TTTGTAATGTTCATAGAAATG | |
| | AGCATGT | |
| PBS10050 | GTGTATGACTGCTTTGTTAAG | HCoV-229E |
| | AATGTGGATTGGTCAATTACC | |
| | TACCCTATGATAGCTAATGAA | |
| | AATGCCA | |
| PBS10051 | TTGCATCTTCTTTTGTTGGTA | HCoV-229E |
| | TGCCATCTTTTGTTGCATATG | |
| | AAACAGCAAGACAAGAGTATG | |
| | AAAATGC | |
| PBS10052 | AAATGGTTCCTCACCACAAAT | HCoV-229E |
| | AATCAAACAATTGAAGAAGGC | |
| | TATGAATGTTGCAAAAGCTGA | |
| | GTTTGAC | |
| PBS10053 | CTGCTGCAGCTATGTACAAAG | HCoV-229E |
| | AAGCACGTGCTGTTAATAGAA | |
| | AATCAAAAGTTGTTAGTGCCA | |
| | TGCATAG | |
| PBS10054 | ACGTTTGGACATGTCTAGTGT | HCoV-229E |
| | TGACACTATCCTTAATATGGC | |
| | ACGTAATGGTGTTGTCCCTCT | |
| | TTCCGTT | |
| PBS10055 | CTGGTGGTAAAGTTTCATTTT | HCoV-229E |
| | CTGATGACGTTGAAGTAAAAG | |
| | ACATTGAACCTGTTTACAGAG | |
| | TCAAGCT | |
| PBS10058 | TTTACAGAGTCAAGCTTTGCT | HCoV-229E |
| | TTGAGTTTGAAGATGAAAAAC | |
| | TTGTAGATGTTTGTGAAAAGG | |
| | CAATTGG | |
| PBS10057 | GATGTTTGTGAAAAGGCAATT | HCoV-229E |
| | GGCAAGAAAATTAAACATGAA | |
| | GGTGACTGGGATAGCTTTTGT | |
| | AAGACTA | |
| PBS10058 | GCGTTGTTGGCCTTTTTCTTG | HCoV-229E |
| | TCTAAGCATAGTGATTTTGGT | |
| | CTTGGTGATCTTGTCGATTCT | |
| | TATTTTG | |
| PBS10059 | AGCAAGACAAGAGTATGAAAA | HCoV-229E |
| | TGCTGTTGCAAATGGTTCCTC | |
| | ACCACAAATAATCAAACAATT | |
| | GAAGAAG | |
| PBS10060 | TTGAAGAAGGCTATGAATGTT | HCoV-229E |
| | GCAAAAGCTGAGTTTGACAGG | |
| | GAATCATCTGTTCAAAAGAAA | |
| | ATTAACA | |
| PBS10061 | CTGCTGCAGCTATGTACAAAG | HCoV-229E |
| | AAGCACGTGCTGTTAATAGAA | |
| | AATCAAAAGTTGTTAGTGCCA | |
| | TGCATAG | |
| PBHE_00001 | CGGGATAAGGCACTCTCTATC | Human enteric |
| | AGAATGGATGTCTTGCTGCTA | coronaviruse |
| | TAATAGATAGAGAAGGTTATA | |
| | GCAGACT | |
| PBHE_00002 | CCCTCGCAGGAAAGTCGGGAT | Human enteric |
| | AAGGCACTCTCTATCAGAATG | coronaviruse |
| | GATGTCTTGCTGCTATAATAG | |
| | ATAGAGA | |
| PBHE_00003 | ATGGATGTTTGAGGACGCAGA | Human enteric |
| | GGAGAAGTTGGACAACCCTAG | coronaviruse |
| | TAGTTCAGAGGTGGATATAGT | |
| | ATGCT | |
| PBHE_00004 | CCTTGGGTTATGTACTTGCGT | Human enteric |
| | AAGTGTGGCGAAAAGGGTGCC | coronaviruse |
| | TACAATAAAGATCATAAACGT | |
| | GTCGG | |
| PBHE_00005 | GGGGATGCTGGTTTTACTAGC | Human enteric |
| | ATACTCAGTGGTTTGTTATAT | coronaviruse |
| | GATTCACCCTGTTTTTCACAG | |
| | CAAGG | |
| PBHE_00006 | CATGACGGCAGTTGCTTGTCA | Human enteric |
| | ACCCCCGTACTGTTATTTTCG | coronaviruse |
| | TAATTCTACTACCAACTATGT | |
| | TGGTG | |
| PBRh_00001 | GGCTGAGTGATTACATCACAG | Human rhinovirus |
| | GTTTGGGTAGAGCTTTTGGTG | |
| | TCGGGTTCACTGACCAAATCT | |
| | CAACAAA | |
| PBRh_00002 | GAAAAGCTATTAGCTTGGTAG | Human rhinovirus |
| | ACAGAACTACCAACGTTAGGT | |
| | ATAGTGTGGATCAACTGGTCA | |
| | CGGCTAT | |
| PBRh_00003 | GGCCAAGTAATAGCTAGACAT | Human rhinovirus |
| | AAGGTTAGGGAGTTTAACATA | |
| | AATCCAGTCAACACGGCAACT | |
| | AAGTCAA | |
| PBRh_00004 | GATAACAAGGGCATGTTATTC | Human rhinovirus |
| | ACCAGTAATTTTGTTCTAGCC | |
| | TCCACAAATTCTAACACACTA | |
| | AGCCCCC | |
| PBRh_00005 | GGCCAAGAAGTAAGGTTGTGT | Human rhinovirus |
| | TTAGTACCACTCAGGGTTTAC | |
| | CAGTTATGTTAACACCTGGAT | |
| | CTGGGCA | |
| PBRh_00006 | GTAATGCGTAAGTGCGGGATG | Human rhinovirus |
| | GGACCAACTACTTTGGGTGTC | |
| | CGTGTTTCCTGTTTTTCTTTT | |
| | GATTGCA | |
| PBRh_00007 | TAAAAGAGGATTCAGAGCTGA | Human rhinovirus |
| | TGAGCGCCACTCTTTCCTTAT | |
| | ACACCCTACCTTTCCTGTGGC | |
| | TGAGATT | |
| PBRh_00008 | GCAAGTTTCATCAGGGTTTAT | Human rhinovirus |
| | TAATAGTTGCCGCCATCCCAG | |
| | AACATCAATTGGCATCTGCAA | |
| | CAAGTGG | |
| PBMP_00001 | ATATATGAAGGAACACCAGTG | Mycoplasma |
| | GCGAAGGCGAAACTTAGGCCA | pneumoniae |
| | TTACTGACGCTTAGGCTTGAA | |
| | AGTGTG | |
| PBMP_00002 | GCAGTAGGGAATTTTTCACAA | Mycoplasma |
| | TGAGCGAAAGCTTGATGGAGC | pneumoniae |
| | AATGCCGCGTGAACGATGAAG | |
| | GTCTTTA | |
| PBMP_00003 | AACACATTAAGTATCTCGCCT | Mycoplasma |
| | GGGTAGTACATTCGCAAGAAT | pneumoniae |
| | GAAACTCAAACGGAATTGACG | |
| | GGGACCC | |
| PBMP_00004 | ACACCGTAAACGATAGATACT | Mycoplasma |
| | AGCTGTCGGGGCGATCCCCTC | pneumoniae |
| | GGTAGTGAAGTTAACACATTA | |
| | AGTATCT | |
| PBMP_00005 | ACATCCTTGGCAAAGTTATGG | Mycoplasma |
| | AAACATAATGGAGGTTAACCG | pneumoniae |
| | AGTGACAGGTGGTGCATGGTT | |
| | GTCGTCA | |
| PBR_00001 | TTATAACTTAACCGTCGGCAG | Rubella virus |
| | TTGGGTAAGAGACCACGTCCG | |
| | ATCAATTGTCGAGGGCGCGTG | |
| | GGAAGTG | |
| PBR_00002 | ATACCCAGACCTGTGTTCACG | Rubella virus |
| | CAGATGCAGGTCAGTGATCAC | |
| | CCAGCACTCCACGCAATTTCG | |
| | CGGTATA | |
| PBR_00003 | AGAAACTCCTAGATGAGGTTC | Rubella virus |
| | TTGCCCCCGGTGGGCCTTATA | |
| | ACTTAACCGTCGGCAGTTGGG | |
| | TAAGAGA | |
| PBR_00004 | ATACCCAGACCTGTGTTCACG | Rubella virus |
| | CAGATGCAGGTCAGTGATCAC | |
| | CCAGCACTCCACGCAATTTCG | |
| | CGGTATA | |
| PBR_00005 | TCTTACTTCAACCCTGGCGGC | Rubella virus |
| | AGCTACTACAAGCAGTACCAC | |
| | CCTACCGCGTGCGAGGTTGAA | |
| | CCT | |
| PBM_00001 | AAGGCTTGTTTCAGAGATTGC | Measles virus |
| | AATGCATACTACTGAGGACAG | |
| | GATCAGTAGAGCAGTTGGACC | |
| | CAGACAA | |
| PBM_00002 | AGGATCAGTAGAGCAGTTGGA | Measles virus |
| | CCCAGACAAGCCCAAGTGTCA | |
| | TTCCTACACGGTGATCAAAGT | |
| | GAGAATG | |
| PBM_00003 | TCAGTAGAGCAGTTGGACCCA | Measles virus |
| | GACAAGCCCAAGTGTCATTCC | |
| | TACACGGTGATCAAAGTGAGA | |
| | ATG | |
| PBM_00004 | CCCAGGGAATGTACGGGGGAA | Measles virus |
| | CTTACCTAGTTGAAAAGCCTA | |
| | ATCTGAGCAGCAAAGGATCAG | |
| | AATTATC | |
| PBM_00005 | CCCAGGGGAATGTACGGGGGA | Measles virus |
| | ACTTACCTAGTTGAAAAGCCT | |
| | AATCTGAGCAGCAAAGGATCA | |
| | GAATTATC | |
| PBRSV_00001 | CAAACCCACAAACAAACCAAC | Human respiratory |
| | CACCAAAACCACAAACAAAAG | syncytial virus |
| | AGACCCAAAAACACCAGCCAA | |
| | AACGACG | |
| PBRSV_00002 | GCAGCACTTGTAATAACCAAA | Human respiratory |
| | TTAGCAGCAGGAGACAGATCA | syncytial virus |
| | GGTCTTACAGCAGTAATTAGG | |
| | AGGGCAA | |
| PBRSV_00003 | CAAGAGGGGGTAGTAGAGTTG | Human respiratory |
| | AAGGAATCTTTGCAGGATTGT | syncytial virus |
| | TTATGAATGCCTATGGTTCAG | |
| | GGCAAGT | |
| PBRSV_00004 | GACTTAACAGCAGAAGAATTG | Human respiratory |
| | GAAGCCATAAAGAATCAACTC | syncytial virus |
| | AACCCTAAAGAAGATGATGTA | |
| | GAGCTTT | |
| PBRSV_00005 | TCACAATCCACTGTGCTCGAC | Human respiratory |
| | ACAACCACATTAGAACACACA | syncytial virus |
| | ATCCAACAGCAATCCCTCCAC | |
| | TCAACCA | |
| PBRSV_00006 | GACTTAACAGCAGAAGAATTG | Human respiratory |
| | GAAGCCATAAAGAATCAACTC | syncytial virus |
| | AACCCTAAAGAAGATGATGTA | |
| | GAGCTTT | |
| PBPI_00001 | GCCGACGACCATCAAGCGTAG | Parainfluenza |
| | CCAAACAAGATCAGAGAGAAC | |
| | ACAGAATTCAGAACTCCACAA | |
| | ATCAACA | |
| PBPI_00002 | CGACCCAAGATCATAGATCAA | Parainfluenza |
| | GTGAGGAGAGTGGAATCTCTA | |
| | GGAGAACAGGTGAGTCAAAAA | |
| | CTGAGAC | |
| PBPI_00003 | CGCAAATGAAGAGGGAACCAG | Parainfluenza |
| | CAACACATCAGTCGATGAGAT | |
| | GGCCAAGTTACTAGTAAGTCT | |
| | TGGTGTA | |
| PBPI_00004 | CTCCTTGCAATGGCCATACGT | Parainfluenza |
| | AGTCCGGAATTATATCTCACT | |
| | ACAAACGGTGTCAATGCTGAT | |
| | GTCAAGT | |
| PBPI_00005 | GAACAAAAACAGATGGGTTCA | Parainfluenza |
| | TTGTCAAAACGAGAGACATGG | |
| | AGTATGAAAGAACCACAGAGT | |
| | GGTTGTT | |
| PBPI_00006 | TGTTCCAAGGGCAAAGAGAGA | Parainfluenza |
| | ATGCGGATCTAGAGGCATTGC | |
| | TTCAGACATATGGATATCCTG | |
| | CATGTCT | |
| PBPI_00007 | GGTATATCCCTCTTCCCAGCC | Parainfluenza |
| | ACATCATGACAAAAGGGGCAT | |
| | TTCTAGGTGGAGCAGATATCA | |
| | AAGAATG | |
| PBPI_00008 | GTATAACAACCACATGTACAT | Parainfluenza |
| | GCAACGGTATTGGCAATAGAA | |
| | TCAATCAACCACCTGATCAAG | |
| | GAGTAAA | |
| PBPI_00009 | CCCAACCCATTCAAAACGAAA | Parainfluenza |
| | ATCTCAAAAGAGATTGGCAAC | |
| | ACAACAAACACTGAACATCAT | |
| | GCCAACC | |
| PBME_00001 | AAAAGTGTATCACAGAAGTTT | Human |
| | GTTCATTGAGTATGGCAAAGC | metapneumovirus |
| | ATTAGGCTCATCATCTACAGG | |
| | CAGCAAA | |
| PBME_00002 | GAAAGTCTATTTGTTAATATA | Human |
| | TTCATGCAAGCTTATGGAGCC | metapneumovirus |
| | GGTCAAACAATGCTAAGGTGG | |
| | GGGGTCA | |
| PBME_00003 | ACGCTGTTGTGTGGAGAAATT | Human |
| | CTGTATGCTAAACATGCTGAT | metapneumovirus |
| | TACAAATATGCTGCAGAAATA | |
| | GGAATAC | |
| PBME_00004 | TTAAGGAATCATCAGGTAATA | Human |
| | TCCCACAAAATCAGAGGCCCT | metapneumovirus |
| | CAGCACCAGACACACCCATAA | |
| | TCTTATT | |
| PBME_00005 | TGAGCAATCAAAGGAGTGCAA | Human |
| | CATCAACATATCCACTACAAA | metapneumovirus |
| | TTACCCATGCAAAGTCAGCAC | |
| | AGGAAGA | |
| PBME_00006 | CTGTTCCATTGGCAGCAACAG | Human |
| | AGTAGGGATCATCAAGCAGCT | metapneumovirus |
| | GAACAAAGGTTGCTCCTATAT | |
| | AACCAAC | |
| PBME_00007 | ACTTAATGACAGATGCTGAAC | Human |
| | TAGCCAGGGCCGTTTCTAACA | metapneumovirus |
| | TGCCGACATCTGCAGGACAAA | |
| | TAAAATT | |
| PBME_00008 | AAAAAAGGGAAACTATGCTTG | Human |
| | CCTCTTAAGAGAAGACCAAGG | metapneumovirus |
| | GTGGTATTGTCAGAATGCAGG | |
| | GTCAAC | |
| PBME_00009 | GAAAAGAACACACCAGTTACA | Human |
| | ATACCAGCATTTATCAAATCG | metapneumovirus |
| | GTTTCTATCAAAGAGAGTGAA | |
| | TCAGCCA | |
| PBME_00010 | CAAATCAGTTGGCAAAAAAAC | Human |
| | ACATGATCTGATCGCATTATG | metapneumovirus |
| | TGATTTTATGGATCTAGAAAA | |
| | GAACACA | |
| PBME_00011 | CAGCTAAAGACACTGACTATA | Human |
| | ACTACTCTGTATGCTGCATCA | metapneumovirus |
| | CAAAGTGGTCCAATACTAAAA | |
| | GTGAATG | |
| PBME_00012 | AAAAGAACACACCAGTTACAA | Human |
| | TACCAGCATTTATCAAATCGG | metapneumovirus |
| | TTTCTATCASAAGAGAGTGAA | |
| | TCAGCCAC | |
| PBME_00013 | CTATTATAGGAGAAAAAGTGA | Human |
| | ACACTGTATCTGAAACATTGG | metapneumovirus |
| | AATTACCTACTATCAGTAGAC | |
| | CCACCAA | |
| PBME_00014 | AAGTTAGCATGGACAGACAAA | Human |
| | GGTGGGGCAATCAAAACTGAA | metapneumovirus |
| | GCAAAGCAAACAATCAAAGTT | |
| | ATGGATC | |
| PBME_00015 | CAGGAAAATACACAAAGTTGG | Human |
| | AGAAAGATGCTCTAGACTTGC | metapneumovirus |
| | TTTCAGACAATGAAGAAGAAG | |
| | ATGCAGA | |
| PBME_00016 | CTAATAGCAGACATAATAAAA | Human |
| | GAAGCCAAGGGAAAAGCAGCA | metapneumovirus |
| | GAAATGATGGAAGAAGAAATG | |
| | AACCAGC | |
| PBCP_00001 | ACCCTTATCGTTAGTTGCCAG | Chlamydophila |
| | CACTTAGGGTGGGAACTCTAA | pneumoniae |
| | CGAGACTGCCTGGGTTAACCA | |
| | GGAGGAA | |
| PBCP_00002 | ATAAGAGAGGTTGGCTAATAT | Chlamydophila |
| | CCAATTGATTTGAGCGTACCA | pneumoniae |
| | GGTAAAGAAGCACCGGCTAAC | |
| | TCCGTGC | |
| PBCP_00003 | CATGGGATCTTAAGTTTTAGT | Chlamydophila |
| | TGAATACTTCTGGAAAGTTGA | pneumoniae |
| | ACGATACAGGGTGATAGTCCC | |
| | GTAAACG | |
| PBCP_00004 | GGGTGCTAGCGTTAATCGGAT | Chlamydophila |
| | TTATTGGGCGTAAAGGGCGTG | pneumoniae |
| | TAGGCGGAAAGGAAAGTTAGA | |
| | TGTTAAA | |
| PBCP_00005 | GCCAGGGAGTTAAGTTAAACG | Chlamydophila |
| | GCGAGATTAAGGGATTTACAT | pneumoniae |
| | TCCGGAGTCGAAGCGAAAGCG | |
| | AGTTTTA | |
| PBCP_00006 | GCCAGGGAGTTAAGTTAAACG | Chlamydophila |
| | GCGAGATTAAGGGATTTACAT | pneumoniae |
| | TCCGGAGTCGAAGCGAAAGCG | |
| | AGTTTTA | |

In some embodiments, the non-SARS-CoV infectious organism is an infectious organism damaging an infectious host's immune system. Such organism includes, but not limited to, a hepatitis virus, a transfusion transmitting virus (TTV), a human immunodeficiency virus (HIV), a parvovirus, a human cytomegalovirus (HCMV), an Epstein-Barr virus (EBV) and a tre-ponema palidum. The hepatitis virus can be hepatitis virus A (HAV), hepatitis virus B (HBV), hepatitis virus C (HCV), hepatitis virus D (HDV), hepatitis virus E (HEV), or hepatitis virus G (HGV). The HIV can be HIV I. The parvovirus can be parvovirus B19. Exemplary probes are set forth in Table 16.

TABLE 16

Exemplary probes for Non-SARS-CoV infectious
organisms damaging host's immune system

| | | |
|---|---|---|
| Id | sequence (5′-3′) | species |

| | | |
|---|---|---|
| PBHAV_00001 | GGTGTTGAACCTGAGAAAAATATTTACAC | HAV |
| | CAAACCTGTGGCCTCAGATTATTGGGATG | |
| | GATATAGTGGAC | |
| PBHAV_00002 | ACTGAGGAGCATGAAATAATGAAGTTTTC | HAV |
| | TTGGAGAGGAGTGACTGCTGATACTAGGG | |
| | CTTTGAGAAGAT | |
| PBHAV_00003 | CATGGCGTGACTAAGCCCAAACAAGTGAT | HAV |
| | TAAATTGGATGCAGATCCAGTAGAGTCCC | |
| | AGTCAACTCTAG | |
| PBHAV_00004 | GTGCAGTGATGGACATTACAGGAGTGCAG | HAV |
| | TCAACCTTGAGATTTCGTGTTCCTTGGAT | |
| | TTCTGATACACC | |
| PBHAV_00005 | CCAAAAGAGATTTAATTTGGTTGGATGAA | HAV |
| | AATGGTTTGCTGTTAGGAGTTCACCCAAG | |
| | ATTGGCCCAGAG | |
| PBHAV_00006 | AGAGATGCTTTGGATAGGGTAACAGCGGC | HAV |
| | GGATATTGGTGAGTTGTTAAGACAAAAAC | |
| | CATTCAACGCCG | |
| PBHBV_00001 | GCTGGATGTGTCTGCGGCGTTTTATCATA | HBV |
| | TTCCTCTTCAATCCTGCTGCTATGCCTCA | |
| | TCTTCTTATTGGT | |
| PBHBV_00002 | ATATACATCCTTTCCATAGCTGCTAGGTT | HBV |
| | GTACTGCCAACTAGATTCTTCGCGGGACG | |
| | TCCTTTGTCTAC | |
| PBHBV_00003 | ATTCTTTCCCGATCATCAGTTGGACCCTG | HBV |
| | CATTCGGAGCCAATTCAAACAATCCAGAT | |
| | TGGGACTTCAAC | |
| PBHBV_00004 | CTCATGTTGCTGTACAAAACCTACGGATG | HBV |
| | GAAATTGCACCTGTATTCCCATCCCATCA | |
| | TCTTGGGCTTTC | |
| PBHBV_00005 | AGAGTCTAGACTCGTGGTGGACTTCTCTC | HBV |
| | AATTTTCTAGGGGGAGCACCCGTGTGTCT | |
| | TGGCCAAAATTC | |
| PBHBV_00006 | TGGGAGACAGCAAGACACACTCCAGTCAA | HBV |
| | TTCCTGGCTAGGCAACATAATCATGTTTG | |
| | CCCCCACACTGT | |
| PBHCV_00001 | TGGGAGACAGCAAGACACACTCCAGTCAA | HCV |
| | TTCCTGGCTAGGCAACATAATCATGTTTG | |
| | CCCCCACACTGT | |
| PBHCV_00002 | TGAGCGACTTTAAGACCTGGCTGAAAGCC | HCV |
| | AAGCTCATGCCACAACTGCCTGGGATTCC | |
| | CTTTGTGT | |
| PBHCV_00003 | TATAGATGCCCACTTTCTATCCCAGACAA | HCV |
| | AGCAGAGTGGGGAGAACTTTCCTTACCTG | |
| | GTAGCGTACCAA | |
| PBHCV_00004 | TAACAACACCAGGCCACCGCTGGGCAATT | HCV |
| | GGTTCGGTTGTACCTGGATGAACTCAACT | |
| | GGATTCACCAAA | |
| PBHCV_00005 | TTTATCCCTGTGGAGAACCTAGAGACAAC | HCV |
| | CATGAGATCCCCGGTGTTCACGGACAACT | |
| | CCTCTCCACCAG | |
| PBHCV_00006 | TTTATCCCTGTGGAGAACCTAGAGACAAC | HCV |
| | CATGAGATCCCCGGTGTTCACGGACAACT | |
| | CCTCTCCACCAG | |
| PBHDV_00001 | TTCCCTTCTCTCGTCTTCCTCGGTCAACC | HDV |
| | TCTTAAGTTCCTCTTCTTCTTCCTTGCTG | |
| | AGGTGCTTCCCT | |
| PBHDV_00002 | TAAGCCCATAGCGATAGGGAGAGATGCTA | HDV |
| | GGAGTTAGAGGAGACCGAAGCGAGGAGGA | |
| | AAGCAAAGAGAG | |
| PBHDV_00003 | TTGGAGAGCACTCCGGCCGAAAGGTCGAG | HDV |
| | GTACCCAGAAGGAGGAATCTCACGGAGAA | |
| | AAGCAGACAAAT | |
| PBHDV_00004 | TTAAGTTCCTCTTCTTCTTCCTTGCTGAG | HDV |
| | GTGCTTCCCTCCCGCGGCCAGCTGCTTTC | |
| | TCTTGTTCTCGA | |
| PBHDV_00005 | AAAAAGAGAAAGCAAGAGACGGACGATTT | HDV |
| | CCCCATGACTCTGGAGACATCCTGGAAGG | |
| | GGAAAGAAGGAA | |
| PBHDV_00006 | AAGTTCCTCTTCTTCTTCCTTGCTGAGGT | HDV |
| | GCTTCCCTCCCGCGGCCAGCTGCTTTCTC | |
| | TTGTTCTCGAGG | |
| PBHGV_00001 | TCATATCATGCATCATTGGACACGGCCCC | HGV |
| | CTTCTGCTCCACTTGGCTTGCTGAGTGCA | |
| | ATGCAGAT | |
| PBHGV_00002 | TAAAGTGGGAAAGTGAGTTTTGGAGATGG | HGV |
| | ACTGAACAGCTGGCCTCCAACTACTGGAT | |
| | TCTGGAATACCT | |
| PBHGV_00003 | TAGGTCGTAAATCCCGGTCACCTTGGTAG | HGV |
| | CCACTATAGGTGGGTCTTAAGAGAAGGTT | |
| | AAGATTCCTCTT | |
| PBHGV_00004 | TTCTTGGTTTGCCTCCACCAGTGGTCGCG | HGV |
| | ACTCGAAGATAGATGTGTGGAGTTTAGTG | |
| | CCAGTTGG | |
| PBHGV_00005 | TCCAACTACTGGATTCTGGAATACCTCTG | HGV |
| | GAAGGTCCCATTTGATTTCTGGAGAGGCG | |
| | TGATAAGCCTGA | |
| PBHGV_00006 | ACGTTACCAAGGTCTTCATGTATCCCGGA | HGV |
| | CAGTTACTTTCAGCAAGTTGACTATTGCG | |
| | ACAAGGTCTCAG | |
| PBTTV_00001 | TGTCAGTAACAGGGGTCGCCATAGACTTC | TTV |
| | GGCCTCCATTTTACCTTGTAAAAACTACC | |
| | AAAATGGCCGTT | |
| PBTTV_00002 | ATGTCATCCATTTCCTGGGCCGGGTCTAC | TTV |
| | GTCCTCATATAAGTAACTGCACTTCCGAA | |
| | TGGCTGAGTTTT | |
| PBTTV_00003 | GGGATCTAGCATCCTTATTTCAAATAGCA | TTV |
| | CCATAAACATGTTTGGTGACCCCAAACCT | |
| | TACAACCCTTCC | |
| PBTTV_00004 | TGTTAGAAATCCCTGCAAAGAAACCCACT | TTV |
| | CCTCGGGCAATAGAGTCCCTAGAAGCTTA | |
| | CAAATCGTTGAG | |
| PBTTV_00005 | TCAAGGATTGACGTAAAGGTTAAAGGTCA | TTV |
| | TCCTCGGCGGAAGCTACACAAAATGGTGG | |
| | ACAACATCTTCC | |
| PBB19_00001 | GGCATGGTTAACTGGAATAATGAAAACTT | B19 |
| | TCCATTTAATGATGTAGCAGGGAAAAGCT | |
| | TGGTGGTCTGGG | |
| PBB19_00002 | GGCAAGAAAAATACACTGTGGTTTTATGG | B19 |
| | GCCGCCAAGTACAGGAAAAACAAACTTGG | |
| | CAATGGCCATTG | |
| PBB19_00003 | GCCATTTCTCATGGTCAGACCACTTATGG | B19 |
| | TAACGCTGAAGACAAAGAGTATCAGCAAG | |
| | GAGTGGGTAGAT | |
| PBB19_00004 | AATTTCGAGAATTTACCCCAGATTTGGTG | B19 |
| | CGGTGTAGCTGCCATGTGGGAGCTTCTAA | |
| | TCCCTTTTCTGT | |
| PBHCMV_00001 | AGGTGCGCAACGCTTTTATGAAGGTAAAG | HCMV |
| | CCCGTGGCCCAGGAGATTATCCGTATCTG | |
| | CATACTCGCTAA | |
| PBHCMV_00002 | TAAACGACATGTATCTGTTGTTGACGCTG | HCMV |
| | CGACACTTGCAGCTGCGACACGCGCTGGA | |
| | GCTACAAATGAT | |
| PBHCMV_00003 | CAAAGCAGCGTCAACAACAGCCACACAGA | HCMV |
| | AACCTACGTGGAGACGACACGGGACTTTT | |
| | TATTGACGGAGA | |
| PBHCMV_00004 | TGCTCCAAAGCAGCGTCAACAACAGCCAC | HCMV |
| | ACAGAAACCTACGTGGAGACGACACGGGA | CTTTTTATTGAC |
| PBEBV_00001 | GAGTTAAAAGCAACTACTGTTTATTTTCC | EBV |
| | AAAATGAGCTGGGTATAGTTGATGATCTG | |
| | TAGGCGCAGCTC | |
| PBEBV_00002 | ACAGTGACAGTGGGAGAAACACGGCCTCT | EBV |
| | GAGACATGTATGGGGGTGTTCATCTCACG | |
| | CAGAAAATCTTT | |
| PBEBV_00003 | TGAAGAAGTCCCGTAGTGAAAAATGGGAT | EBV |
| | CTGTCTACACCATGTCTGGTGTGCCGGGA | |
| | ACATATTGATCG | |
| PBEBV_00004 | TGAAGAAGTCCCGTAGTGAAAAATGGGAT | EBV |
| | CTGTCTACACCATGTCTGGTGTGCCGGGA | |
| | ACATATTGATCG | |
| PBHIV1_00001 | ATTATTGTCTGGTATAGTGCAGCAGCAGA | HIV1 |
| | ACAATTTGCTGAGGGCTATTGAGGCGCAA | |
| | CAGCATCTGTTG | |
| PBHIV1_00002 | GCAACCCTCTATTGTGTGCATCAAAGGAT | HIV1 |
| | AGAGATAAAAGACACCAAGGAAGCTTTAG | |
| | ACAAGATAGAGG | |
| PBHIV1_00003 | TGTATGTAGGATCTGACTTAGAAATAGGG | HIV1 |
| | CAGCATAGAACAAAAATAGAGGAGCTGAG | |
| | ACAACATCTGTT | |
| PBHIV1_00004 | GGAATGCTAGTTGGAGTAATAAATCTCTG | HIV1 |
| | GAACAGATTTGGAATCACACGACCTGGAT | |
| | GGAGTGGGACAG | |
| PBTP_00001 | TACCTTGAAAGACGTTACCGCCAAAATGC | TP |
| | TCATCAAAAGAACGAGGACCATGCTGACA | |
| | GCACCCGCGACA | |
| PBTP_00002 | TTTCGTGATCCTTTTCCTTTTCCTGTAGC | TP |
| | TCAGCGTCCTTTTTATCTAATTCCTCTGC | |
| | ACGCTCCCCGAG | |
| PBTP_00003 | TCTTTCTGACTCGCGCAAAAGGCATTACT | TP |
| | GGAACACTATTTTAGCCATGTGGTGGCTC | |
| | CCTGCTATCTTA | |
| PBTP_00004 | ACCTTGAAAGACGTTACCGCCAAAATGCT | TP |
| | CATCAAAAGAACGAGGACCATGCTGACAG | |
| | CACCCGCGACAA | |
| PBHEV_00001 | AATAATTCACGCCGTCGCTCCTGATTATA | HEV |
| | GGTTGGAACATAACCCAAAGATGCTTGAG | |
| | GCTGCCTACCGG | |
| PBHEV_00002 | TTTGTTGACGGGGCGGTTTTAGAGACTAA | HEV |
| | TGGCCCAGAGCGCCACAATCTCTCTTTTG | |
| | ATGCCAGTCAGA | |
| PBHEV_00003 | ATTTTACTAGTACTAATGGTGTCGGTGAG | HEV |
| | ATCGGCCGCGGGATAGCGCTTACCCTGTT | |
| | TAACCTTGCTGA | |
| PBHEV_00004 | AGTCCACTTACGGCTCTTCGACCGGCCCA | HEV |
| | GTCTATGTCTCTGACTCTGTGACCTTGGT | |
| | TAATGTAG | |

In some embodiments, the non-SARS-CoV infectious organism is a non-SARS-CoV coronaviridae virus. Such virus includes, but not limited to, an avian infectious bronchitis virus, an avian infectious laryngotracheitis virus, a murine hepatitis virus, an equine coronaviruse, a canine coronaviruse, a feline coronaviruse, a porcine epidemic diarrhea virus, a porcine transmissible gastroenteritis virus, a bovine coronaviruse, a feline infectious peritonitis virus, a rat coronaviruse, a neonatal calf diarrhea coronaviruse, a porcine hemagglutinating encephalomyelitis virus, a puffinosis virus, a turkey coronaviruse and a sialodacryoadenitis virus of rat. Exemplary probes for these viruses are set forth in Table 17.

TABLE 17

Exemplary probes for non-SARS-CoV
coronaviridae virus

| | |
|---|---|
| seqid | sequence (5′-3′) |

| | |
|---|---|
| PBIBV_00001 | GGTATAGTGTGGGTTGCTGCTAAGGGTGCTGATACTA |
| | AATCTAGATCCAATCAGGGTACAAGAGATCCTG |
| PBIBV_00002 | GGTATAGTGTGGGTTGCTGCTAAGGGTGCTGATACTA |
| | AATCTAGATCCAATCAGGGTACAAGAGATCCTG |
| PBMHV_00001 | CCAGCCCAAGCAAGTAACGAAGCAAAGTGCCAAAGAA |
| | GTCAGGCAGAAAATTTTAAACAAGCCTCGCCAA |
| PBMHV_00002 | TCTAAACTTTAAGGATGTCTTTTGTTCCTGGGCAAGA |
| | AAATGCCGGTGGCAGAAGCTCCTCTGTAAACCG |
| PBEQ_00001 | AGGATCAAGAAATAGATCCAATTCCGGCACTAGAACA |
| | CCCACCTCTGGTGTGACATCTGATATGGCTGAT |
| PBEQ_00002 | TTTAAAACAGCCGATGGCAATCAACGCCAATTGTTGC |
| | CACGCTGGTATTTTTACTACTTGGGAACAGGCC |
| PBCA_00001 | TTGGAACTTATGTCCGAGAGACTTTGTACCCAAAGGA |
| | ATAGGTAACAAGGATCAACAGATTGGTTATTGG |
| PBCA_00002 | GCTGAATGTGTTCCATCTGTATCTAGCATTCTGTTTG |
| | GAAGCTATTGGACTGCAAAGGAAGATGGCGACC |
| PBFE_00001 | CACCACCCTCGAACAAGGAGCTAAATTTTGGTATGTA |
| | TGTCCGAGAGACTTTGTTCCCAAGGGAATAGGT |
| PBFE_00002 | GGCACTCGTGGAACCAACAATGAATCCGAACCATTGA |
| | GATTTGATGGTAAGATACCACCACAATTCCAGC |
| PBPEDV_00001 | CTGATCCAAATGTTGAGCTTCTTGTTGCACAGGTGGA |
| | TGCATTTAAAACTGGGAATGCAAAACCCCAGAG |
| PBPEDV_00002 | ATGAGCAAATTCGCTGGCGTATGCGCCGTGGTGAGCG |
| | AATTGAACAACCTTCAAATTGGCATTTCTACTA |
| PBPTGV_00001 | GAGAGACTTTGTACCCAAAGGAATAGGTAACAGGGAT |
| | CAACAGATTGGTTATTGGAATAGACAAACTCGC |
| PBPTGV_00002 | GATGGTGACCAGATAGAAGTCACGTTCACACACAAAT |
| | ACCACTTGCCAAAGGATGATCCTAAAACTGGAC |
| PBBOV_00001 | TATTTTTACTATCTTGGAACAGGACCGCATGCCAAAG |
| | ACCAGTATGGCACCGACATTGACGGAGTCTACT |
| PBBOV_00002 | AGAACCCCTACCTCTGGTGTAACACCTGATATGGCTG |
| | ATCAAATTGCTAGTCTTGTTCTGGCTAAACTTG |
| PBFIPV_00001 | GAGTGTGGTTAATCAACAGGGTGAAGCGCTGAGTCAA |
| | CTTACCAGTCAGTTACAGAAAAACTTCCAGGCT |
| PBFIPV_00002 | CCGGCATTGTAGATGGTAATAAGATGGCCATGTACAC |
| | AGCATCTTTAATTGGAGGTATGGCTTTGGGCTC |
| PBR_00001 | AAATGTTAAAACTTGGAACTAGTGATCCACAGTTCCC |
| | CATTCTTGCAGAGTTGGCCCCAACACCTGGTGC |
| PBR_00002 | CCCATTACTCTTGGTTTTCGGGCATTACCCAATTTCA |
| | AAAGGGAAAGGAGTTCCAGTTTGCAGATGGGCA |
| PBPHEV_00001 | TAGTAACCAGGCTGATATTAATACCCCGGCTGACATT |
| | GTCGATCGGGATCCAAGTAGCGATGAGGCTATT |
| PBPHEV_00002 | TTCTTTTAAAACAGCCGATGGCAATCAGCGTCAACTG |
| | CTGCCACGATGGTACTTTTACTACCTGGGAACA |
| PBPV_00001 | GTGGTTCCCCATTACTCCTGGTTTTCTGGCATTACCC |
| | AATTCCAGAAGGGAAAGGAGTTTAAGTTTGCAG |
| PBPV_00002 | AAGAAGTCAGGCAGAAAATTTTAAACAAGCCTCGCCA |
| | AAAGAGGACTCCAAACAAGCAGTGCCCAGTGCA |
| PBTK_00001 | TTTGGTGATGACAAGATGAATGAGGAAGGTATTAAGG |
| | ATGGGCGTGTTACGGCAATGCTCAACCTAGTCC |
| PBTK_00002 | TTTGGTGATGACAAGATGAATGAGGAAGGTATTAAGG |
| | ATGGGCGTGTTACGGCAATGCTCAACCTAGTCC |
| PBSDAV_00001 | AGCCTGCCTCTACTGTAAAACCTGATATGGCCGAAGA |
| | AATTGCTGCTCTTGTTTTGGCTAAGCTAGGCAA |
| PBSDAV_00002 | CCCCATTCTTGCAGAGTTGGCCCCAACACCTGGTGCC |
| | TTCTTCTTTGGATCTAAATTAGAATTGGTCAAA |

The oligonucleotide probes and the target SARS-CoV and any non-SARS-CoV infectious organism nucleotide sequences can be any suitable length. Preferably, the oligonucleotide probes and the target SARS-CoV and any non-SARS-CoV infectious organism nucleotide sequences have a length of at least 7, 10, 20, 30, 40, 50, 60, 80, 90, 100 or more than 100 nucleotides.

The oligonucleotide probes and primers can be prepared by any suitable methods, e.g., chemical synthesis, recombinant methods and/or both (See generally, Ausubel et al., (Ed.), Current Protocols in Molecular Biology, John Wiley & Sons, Inc. (2000)).

Any suitable support can be used in the present chips. For example, the support can comprise a surface that is selected from the group consisting of a silicon, a plastic, a glass, a ceramic, a rubber, and a polymer surface.

C. Methods for Assaying for a SARS-CoV and a Non-SARS-CoV Infectious Organism

In another aspect, the present invention is directed to a method for assaying for a SARS-CoV and a non-SARS-CoV infectious organism in a sample, which methods comprises: a) providing an above-described chip; b) contacting said chip with a sample containing or suspected of containing a nucleotide sequence of a SARS-CoV and a non-SARS-CoV infectious organism under conditions suitable for nucleic acid hybridization; and c) assessing hybrids formed between said nucleotide sequence of said SARS-CoV or said non-SARS-CoV infectious organism, if present in said sample, and said oligonucleotide probe complementary to a nucleotide sequence of said SARS-CoV genome or said oligonucleotide probe complementary to a nucleotide sequence of said non-SARS-CoV infectious organism genome, whereby detection of one or both of said hybrids indicates the presence of said SARS-CoV and/or said non-SARS-CoV infectious organism in said sample.

In some embodiments, the SARS-CoV is assayed by: a) providing a chip comprising a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotide; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample, whereby detection of one or both said hybrids indicates the presence of said SARS-CoV in said sample.

In a specific embodiment, the present methods comprise: a) providing a chip comprising a nucleotide sequence of at least 10 nucleotides located within a conserved region of SARS-CoV genome and a nucleotide sequence of at least 10 nucleotides located within a variable region of SARS-CoV genome, or a nucleotide sequence of at least 10 nucleotides located within a structural protein coding gene of SARS-CoV genome and a nucleotide sequence of at least 10 nucleotides located within a non-structural protein coding gene of SARS-CoV genome; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and i) said oligonucleotide probe complementary to a nucleotide sequence located within a conserved region of SARS-CoV genome and an oligonucleotide probe complementary to a nucleotide sequence located within a variable region of SARS-CoV genome, respectively; or ii) said oligonucleotide probe complementary to a nucleotide sequence located within a structural protein coding gene of SARS-CoV genome and an oligonucleotide probe complementary to a nucleotide sequence located within a non-structural protein coding gene of SARS-CoV genome, to determine the presence, absence or amount of said SARS-CoV in said sample, whereby detection of one or both said hybrids indicates the presence of said SARS-CoV in said sample.

In another specific embodiment, the present methods comprise: a) providing a chip comprising an oligonucleotide probe complementary to a nucleotide sequence within a conserved region of SARS-CoV genome, an oligonucleotide probe, complementary to a nucleotide sequence located within a variable region of SARS-CoV genome, at least one of the following three oligonucleotide probes: an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample, and a blank spot; b) contacting said chip with a sample containing or suspected of containing a SARS-CoV nucleotide sequence under conditions suitable for nucleic acid hybridization; and c) assessing: (i) hybrids formed between said SARS-CoV nucleotide sequence, if present in the sample, and the oligonucleotide probe complementary to a nucleotide sequence within a conserved region of SARS-CoV genome and an oligonucleotide probe complementary to a nucleotide sequence located within a variable region of SARS-CoV genome, respectively; (ii) a label comprised in the immobilization control probe, or a hybrid(s) involving the positive control probe and/or the negative control probe; and (iii) a signal at said blank spot to determine the presence, absence or amount of said SARS-CoV in a sample.

Preferably, the present chips comprise two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe, a positive control probe and a negative control probe and the presence of the SARS-CoV is determined when: a) a positive hybridization signal is detected using at least one of the two different nucleotide sequences located within the Replicase 1 A or 1B gene of SARS-CoV, the oligonucleotide probe complementary to a nucleotide sequence located within the N gene of SARS-CoV and the oligonucleotide probe complementary to a nucleotide sequence located within the S gene of SARS-CoV; b) a positive signal is detected from the immobilization control probe; c) a positive hybridization signal is detected using the positive control probe; d) a positive hybridization signal is not detected using the negative control probe; and e) a positive hybridization signal is not detected at the blank spot.

The inclusion of a target sequence in a variable region of SARS-CoV enables an assessment of possible mutation of the SARS-CoV. For example, detecting a positive hybridization signal using at least one of the two different nucleotide sequences located within the Replicase 1A or 1B gene of SARS-CoV, or the oligonucleotide probe complementary to a nucleotide sequence located within the N gene of SARS-CoV, while not detecting a positive hybridization signal using the oligonucleotide probe complementary to a nucleotide sequence located within the S gene of SARS-CoV indicates a mutation(s) of the SARS-CoV.

The present methods can be used for any suitable prognosis and diagnosis purpose. In one example, the present method is used to positively identify SARS-CoV infected patients from a population of patients who have SARS-like symptoms, e.g., fever or elevated temperature, nonproductive cough, myalgia, dyspnea, elevated lactate dehydrogenase, hypocalcemia, and lymphopenia (Booth et al., JAMA, 2003 May 6; [epub ahead of print]). The present chips, methods and kits can further comprise assaying for elevated lactate dehydrogenase, hypocalcemia, and lymphopenia, etc.

In another example, a chip further comprising an oligonucleotide probe complementary to a nucleotide sequence of a coronaviruse not related to the SARS-CoV is used and the method is used to positively identify SARS-CoV infected patients from patients who have been infected with a coronaviruse not related to the SARS, e.g., a coronaviruse that infects an avian species, e.g., Avian infectious bronchitis virus and Avian infectious laryngotracheitis virus, an equine species, e.g., Equine coronaviruse, a canine species, e.g., Canine coronaviruse, a feline species, e.g., Feline coronaviruse and Feline infectious peritonitis virus, a porcine species, e.g., Porcine epidemic diarrhea virus, Porcine transmissible gastroenteritis virus and Porcine hemagglutinating encephalomyelitis virus, a calf species, e.g., Neonatal calf diarrhea coronaviruse, a bovine species, e.g., Bovine coronaviruse, a murine species, e.g., Murine hepatitis virus, a puffinosis species, e.g., Puffinosis virus, a rat species, e.g., Rat coronaviruse and a Sialodacryoadenitis virus of rat, e.g., a turkey species e.g., Turkey coronaviruse, or a human species, e.g., Human enteric coronaviruse.

In still another example, a chip comprising an oligonucleotide probes complementary to a highly expressed nucleotide sequence of SARS-CoV genome is used and the method is used to diagnose early-stage SARS patients, e.g., SARS patients who have been infected with SARS-CoV from about less than one day to about three days.

In yet another example, the present methods are used to monitor treatment of SARS, e.g., treatment with an interferon or an agent that inhibits the replication of a variety of RNA viruses such as ribavirin. The present methods can also be used to assess potential anti-SARS-CoV agent in a drug screening assay.

The method of the invention can be used to determine whether a subject is infected by a SARS-CoV and/or a non-SARS-CoV infectious organism causing SARS-like symptoms. Non-SARS-CoV infectious organism that causing SARS-like symptoms includes, but not limited to, a human coronaviruse 229E, a human coronaviruse OC43, a human enteric coronaviruse, an influenza virus, a parainfluenza virus, a respiratory sncytical virus, a human metapneumovirus, a rhinovirus, an adenoviruse, a mycoplasma pneumoniae, a chlamydia pneumoniae, a measles virus and a rubella virus. The influenza virus can be influenza virus A or influenza virus B. The parainfluenza virus can be parainfluenza virus 1, parainfluenza virus 2, parainfluenza virus 3 or parainfluenza virus 4.

The method of the invention can also be used to determine whether a subject is infected by a SARS-CoV and/or a non-SARS-CoV infectious organism damaging the subject's immune system. The non-SARS-CoV infectious organism damaging subject's immune system includes, but not limited to, a hepatitis virus, a transfusion transmitting virus (TTV), a human immunodeficiency virus (HIV), a parvovirus, a human cytomegalovirus (HCMV), an Epstein-Barr virus (EBV) and a tre-ponema palidum. The hepatitis virus can be hepatitis virus A (HAV), hepatitis virus B (HBV), hepatitis virus C (HCV), hepatitis virus D (HDV), hepatitis virus E (HEV), or hepatitis virus G (HGV). The HIV can be HIV I. The parvovirus can be parvovirus B19.

The method of the invention can also be used to determine whether a subject is infected by a SARS-CoV and/or a non-SARS-CoV coronaviridae virus. The non-SARS-CoV coronaviridae virus includes, but not limited to, an avian infectious bronchitis virus, an avian infectious laryngotracheitis virus, a murine hepatitis virus, an equine coronaviruse, a canine coronaviruse, a feline coronaviruse, a porcine epidemic diarrhea virus, a porcine transmissible gastroenteritis virus, a bovine coronaviruse, a feline infectious peritonitis virus, a rat coronaviruse, a neonatal calf diarrhea coronaviruse, a porcine hemagglutinating encephalomyelitis virus, a puffinosis virus, a turkey coronaviruse and a sialodacryoadenitis virus of rat.

Any suitable SARS-CoV or non-SARS-CoV infectious organism nucleotide sequence can be assayed. For example, the SARS-CoV or the non-SARS-CoV infectious organism nucleotide sequence to be assayed can be a SARS-CoV RNA or a non-SARS-CoV infectious organism genomic sequence or a DNA sequence amplified from an extracted SARS-CoV RNA or a non-SARS-CoV infectious organism genomic sequence.

The SARS-CoV RNA or the non-SARS-CoV infectious organism genomic sequence can be prepared by any suitable methods. For example, the SARS-CoV RNA or the non-SARS-CoV infectious organism genomic sequence can be extracted from a SARS-CoV or the non-SARS-CoV infectious organism infected cell or other materials using the QIAamp Viral RNA kit, the Chomczynski-Sacchi technique or TRIzol (De Paula et al., J. Virol. Methods, 98(2):119-25 (2001)). Preferably, the SARS-CoV RNA or the non-SARS-CoV infectious organism genomic sequence is extracted from a SARS-CoV or the non-SARS-CoV infectious organism infected cell or other materials using the QIAamp Viral RNA kit. The SARS-CoV RNA or the non-SARS-CoV infectious organism genomic sequence can be extracted from any suitable source. For example, the SARS-CoV RNA or the non-SARS-CoV infectious organism genomic sequence can be extracted from a sputum or saliva sample. In another example, the SARS-CoV RNA or the non-SARS-CoV infectious organism genomic sequence can be extracted from a lymphocyte of a blood sample.

The SARS-CoV RNA or the non-SARS-CoV infectious organism genomic sequence can be amplified by any suitable methods, e.g., PCR. Preferably, a label is incorporated into the amplified DNA sequence during the PCR. Any suitable PCR can be used, e.g., conventional, multiplex, nested PCR or RT-PCR. In one example, the PCR can comprise a two-step nested PCR, the first step being a RT-PCR and the second step being a conventional PCR. In another example, the PCR can comprise a one-step, multiplex RT-PCR using a plurality of 5′ and 3′ specific primers, each of the specific primers comprising a specific sequence complementary to its target sequence to be amplified and a common sequence, and a 5′ and a 3′ universal primer, the 5′ universal primer being complementary to the common sequence of the 5′ specific primers and the 3′ universal primer being complementary to the common sequence of the 3′ specific primers, and wherein in the PCR, the concentration of the 5′ and 3′ universal primers equals to or is higher than the concentration of the 5′ and 3′ specific primers, respectively. Preferably, the 3′ universal primer and/or the 5′ universal primer is labeled, e.g., a fluorescent label. In still another example, the PCR comprises a multiple step nested PCR or RT-PCR. In yet another example, the PCR is conducted using at least one of the following pairs of primers for SARS-CoV set forth in Table 18.

TABLE 18
Exemplary SARS-CoV primers

| | | |
|---|---|---|
| id | sequence (5′-3′) | region |

| | | |
|---|---|---|
| PMSL_00005 | CACGTCTCCCAAATGCTTGAGTGACG | SARS-Cov Nucleocapsid gene |
| PMSU_00006 | CCTCGAGGCCAGGGCGTTCC | SARS-Cov Nucleocapsid gene |
| PMV_00039 | TCACTTGCTTCCGTTGAGGTCGGGGACCAAGACCTAATCAGA | SARS-Cov Nucleocapsid gene |
| PMV_00040 | GGTTTCGGATGTTACAGCGTAGCCGCAGGAAGAAGAGTCACAG | SARS-Cov Nucleocapsid gene |
| PMV_00041 | TCACTTGCTTCCGTTGAGGAGGCCAGGGCGTTCCAATC | SARS-Cov Nucleocapsid gene |
| PMV_00042 | GGTTTCGGATGTTACAGCGTCAATAGCGCGAGGGCAGTTTC | SARS-Cov Nucleocapsid gene |
| PMV_00043 | TCACTTGCTTCCGTTGAGGGGCACCCGCAATCCTAATAACAA | SARS-Cov Nucleocapsid gene |
| PMV_00044 | GGTTTCGGATGTTACGCGTAGCCGCAGGAAGAAGAGTCACAG | SARS-Cov Nucleocapsid gene |
| PMV_00090 | TCGGGGACCAAGACCTAATCAGA | SARS-Cov Nucleocapsid gene |
| PMV_00091 | AGCCGCAGGAAGAAGAGTCACAG | SARS-Cov Nucleocapsid gene |
| PMV_00092 | AGGCCAGGGCGTTCCAATC | SARS-Cov Nucleocapsid gene |
| PMV_00093 | CAATAGCGCGAGGGCAGTTTC | SARS-Cov Nucleocapsid gene |
| PMV_00094 | GGCACCCGCAATCCTAATAACAA | SARS-Cov Nucleocapsid gene |
| PMV_00095 | AGCCGCAGGAAGAAGAGTCACAG | SARS-Cov Nucleocapsid gene |
| PMSL_00001 | ACATCACAGCTTCTACACCCGTTAAGGT | SARS-Cov Replicase 1A |
| PMSL_00002 | ATACAGAATACATAGATTGCTGTTATCC | SARS-Cov Replicase 1A |
| PMSL_00002 | GCATCGTTGACTATGGTGTCCGATTCT | SARS-Cov Replicase 1A |
| PMSU_00003 | GCTGCATTGGTTTGTTATATCGTTATGC | SARS-Cov Replicase 1A |
| PMV_00023 | TCACTTGCTTCCGTTGAGGAGCCGCTTGTCACAATGCCAATT | SARS-Cov Replicase 1A |
| PMV_00024 | GGTTTCGGATGTTACAGCGTCATCACCAAGCTCGCCAACAGTT | SARS-Cov Replicase 1A |
| PMV_00025 | TCACTTGCTTCCGTTGAGGAGGTTGCCATCATTTTGGCATCTT | SARS-Cov Replicase 1A |
| PMV_00026 | GGTTTCGGATGTTACAGCGTCTTTGCGCCAGCGATAGTGACTT | SARS-Cov Replicase 1A |
| PMV_00027 | TCACTTGCTTCCGTTGAGGATGGCACCCGTTTCTGCAATGG | SARS-Cov Replicase 1A |
| PMV_00028 | GGTTTCGGATGTTACAGCGTTCGGGCAGCTGACACGAATGTAGA | SARS-Cov Replicase 1A |
| PMV_00029 | TCACTTGCTTCCGTTGAGGGAATGGCGATGTAGTGGCTATTGA | SARS-Cov Replicase 1A |
| PMV_00030 | GGTTTCGGATGTTACAGCGTTAATGCCGGCATCCAAACATAAT | SARS-Cov Replicase 1A |
| PMV_00031 | TCACTTGCTTCCGTTGAGGTAGCCAGCGTGGTGGTTCATACAA | SARS-Cov Replicase 1A |
| PMV_00032 | GGTTTCGGATGTTACAGCGTCTCCCGGCAGAAAGCTGTAAGCT | SARS-Cov Replicase 1A |
| PMV_00033 | TCACTTGCTTCCGTTGAGGTATAGAGCCCGTGCTGGTGATGC | SARS-Cov Replicase 1A |
| PMV_00034 | GGTTTCGGATGTTACAGCGTATCGCCATTCAAGTCTGGGAAGAA | SARS-Cov Replicase 1A |
| PMV_00035 | TCACTTGCTTCCGTTGAGGTGGCTCAGGCCATACTGGCATTAC | SARS-Cov Replicase 1A |
| PMV_00036 | GGTTTCGGATGTTACAGCGTTTTGCGCCAGCGATAGTGACTTG | SARS-Cov Replicase 1A |
| PMV_00037 | TCACTTGCTTCCGTTGAGGTTCCCGTCAGGCAAAGTTGAAGG | SARS-Cov Replicase 1A |
| PMV_00038 | GGTTTCGGATGTTACAGCGTGACGGCAATTCCTGTTTGAGCAGA | SARS-Cov Replicase 1A |
| PMV_00074 | AGCCGCTTGTCACAATGCCAATT | SARS-Cov Replicase 1A |
| PMV_00075 | CATCACCAAGCTCGCCAACAGTT | SARS-Cov Replicase 1A |
| PMV_00076 | AGGTTGCCATCATTTTGGCATCTT | SARS-Cov Replicase 1A |
| PMV_00077 | CTTTGCGCCAGCGATAGTGACTT | SARS-Cov Replicase 1A |
| PMV_00078 | ATGGCACCCGTTTCTGCAATGG | SARS-Cov Replicase 1A |
| PMV_00079 | TCGGGCAGCTGACACGAATGTAGA | SARS-Cov Replicase 1A |
| PMV_00080 | GAATGGCGATGTAGTGGCTATTGA | SARS-Cov Replicase 1A |
| PMV_00081 | TAATGCCGGCATCCAAACATAAT | SARS-Cov Replicase 1A |
| PMV_00082 | TAGCCAGCGTGGTGGTTCATACAA | SARS-Cov Replicase 1A |
| PMV_00083 | CTCCCGGCAGAAAGCTGTAAGCT | SARS-Cov Replicase 1A |
| PMV_00084 | TATAGAGCCCGTGCTGGTGATGC | SARS-Cov Replicase 1A |
| PMV_00085 | ATCGCCATTCAAGTCTGGGAAGAA | SARS-Cov Replicase 1A |
| PMV_00086 | TGGCTCAGGCCATACTGGCATTAC | SARS-Cov Replicase 1A |
| PMV_00087 | TTTGCGCCAGCGATAGTGACTTG | SARS-Cov Replicase 1A |
| PMV_00088 | TTCCCGTCAGGCAAAGTTGAAGG | SARS-Cov Replicase 1A |
| PMV_00089 | GACGGCAATTCCTGTTTGAGCAGA | SARS-Cov Replicase 1A |
| PMV_00003 | TCACTTGCTTCCGTTGAGGATGAATTACCAAGTCAATGGTTAC | SARS-Cov Replicase 1B |
| PMV_00004 | GGTTTCGGATGTTACAGCGTATAACCAGTCGGTACAGCTAC | SARS-Cov Replicase 1B |
| PMV_00005 | TCACTTGCTTCCGTTGAGGGAAGCTATTCGTCACGTTCG | SARS-Cov Replicase 1B |
| PMV_00006 | GGTTTCGGATGTTACAGCGTCTGTAGAAAATCCTAGCTGGAG | SARS-Cov Replicase 1B |
| PMV_00007 | TCACTTGCTTCCGTTGAGGCCTCTCTTGTTCTTGCTCGCA | SARS-Cov Replicase 1B |
| PMV_00008 | GGTTTCGGATGTTACAGCGTGTGAGCCGCCACACATG | SARS-Cov Replicase 1B |
| PMV_00009 | TCACTTGCTTCCGTTGAGGCTAACATGCTTAGGATAATGG | SARS-Cov Replicase 1B |
| PMV_00010 | GGTTTCGGATGTTACAGCGTCAGGTAAGCGTAAAACTCATC | SARS-Cov Replicase 1B |
| PMV_00011 | TCACTTGCTTCCGTTGAGGGCCTCTCTTGTTCTTGCTCGC | SARS-Cov Replicase 1B |
| PMV_00013 | TCACTTGCTTCCGTTGAGGCACCGTTTCTACAGGTTAGCTAACGA | SARS-Cov Replicase 1B |
| PMV_00014 | GGTTTCGGATGTTACAGCGTAAATGTTTACGCAGGTAAGCGTAAAA | SARS-Cov Replicase 1B |
| PMV_00015 | TCACTTGCTTCCGTTGAGGTACACACCTCAGCGTTG | SARS-Cov Replicase 1B |
| PMV_00016 | GGTTTCGGATGTTACAGCGTCACGAACGTGACGAAT | SARS-Cov Replicase 1B |
| PMV_00017 | TCACTTGCTTCCGTTGAGGGCTTAGGATAATGGCCTCTC | SARS-Cov Replicase 1B |
| PMV_00018 | GGTTTCGGATGTTACAGCGTCCACGAATTCATGATCAACATCCC | SARS-Cov Replicase 1B |
| PMV_00019 | TCACTTGCTTCCGTTGAGGGCTCGCAAACATAACACTTGC | SARS-Cov Replicase 1B |
| PMV_00020 | GGTTTCGGATGTTACAGCGTGAGACACTCATAGAGCCTGTG | SARS-Cov Replicase 1B |
| PMV_00055 | ATGAATTACCAAGTCAATGGTTAC | SARS-Cov Replicase 1B |
| PMV_00056 | ATAACCAGTCGGTACAGCTAC | SARS-Cov Replicase 1B |
| PMV_00057 | GAAGCTATTCGTCACGTTCG | SARS-Cov Replicase 1B |
| PMV_00058 | CTGTAGAAAATCCTAGCTGGAG | SARS-Cov Replicase 1B |
| PMV_00059 | CCTCTCTTGTTCTTGCTCGCA | SARS-Cov Replicase 1B |
| PMV_00060 | GTGAGCCGCCACACATG | SARS-Cov Replicase 1B |
| PMV_00061 | CTAACATGCTTAGGATAATGG | SARS-Cov Replicase 1B |
| PMV_00062 | CAGGTAAGCGTAAAACTCATC | SARS-Cov Replicase 1B |
| PMV_00063 | GCCTCTCTTGTTCTTGCTCGC | SARS-Cov Replicase 1B |
| PMV_00064 | CACCGTTTCTACAGGTTAGCTAACGA | SARS-Cov Replicase 1B |
| PMV_00065 | AAATGTTTACGCAGGTAAGCGTAAAA | SARS-Cov Replicase 1B |
| PMV_00066 | TACACACCTCAGCGTTG | SARS-Cov Replicase 1B |
| PMV_00067 | CACGAACGTGACGAAT | SARS-Cov Replicase 1B |
| PMV_00068 | GCTTAGGATAATGGCCTCTC | SARS-Cov Replicase 1B |
| PMV_00069 | CCACGAATTCATGATCAACATCCC | SARS-Cov Replicase 1B |
| PMV_00070 | GCTCGCAAACATAACACTTGC | SARS-Cov Replicase 1B |
| PMV_00071 | GAGACACTCATAGAGCCTGTG | SARS-Cov Replicase 1B |
| PMSL_00003 | CCAGCTCCAATAGGAATGTCGCACTC | SARS-Cov Spike glycoprotein gene |
| PMSL_00004 | TCCGCAGATGTACATATTACAATCTACG | SARS-Cov Spike glycoprotein gene |
| PMSU_00005 | TTAAATGCACCGGCCACGGTTTG | SARS-Cov Spike glycoprotein gene |
| PMV_000100 | ATAGCGCCAGGACAAACTGGTGTT | SARS-Cov Spike glycoprotein gene |
| PMV_000101 | TATATGCGCCAAGCTGGTGTGAGT | SARS-Cov Spike glycoprotein gene |
| PMV_000102 | CGAGGCGGAGGTACAAATTGACAG | SARS-Cov Spike glycoprotein gene |
| PMV_000103 | ATGAAGCCGAGCCAAACATACCAA | SARS-Cov Spike glycoprotein gene |
| PMV_00045 | TCACTTGCTTCCGTTGAGGATGCACCGGCCACGGTTTGTG | SARS-Cov Spike glycoprotein gene |
| PMV_00046 | GGTTTCGGATGTTACAGCGTATGCGCCAAGCTGGTGTGAGTTGA | SARS-Cov Spike glycoprotein gene |
| PMV_00047 | TCACTTGCTTCCGTTGAGGTGCTGGCGCTGCTCTTCAAATACC | SARS-Cov Spike glycoprotein gene |
| PMV_00048 | GGTTTCGGATGTTACAGCGTCGGGGCTGCTTGTGGGAAGG | SARS-Cov Spike glycoprotein gene |
| PMV_00049 | TCACTTGCTTCCGTTGAGGATAGCGCCAGGACAAACTGGTGTT | SARS-Cov Spike glycoprotein gene |
| PMV_00050 | GGTTTCGGATGTTACAGCGTTATATGCGCCAAGCTGGTGTGAGT | SARS-Cov Spike glycoprotein gene |
| PMV_00051 | TCACTTGCTTCCGTTGAGGCGAGGCGGAGGTACAAATTGACAG | SARS-Cov Spike glycoprotein gene |
| PMV_00052 | GGTTTCGGATGTTACAGCGTATGAAGCCGAGCCAAACATACCAA | SARS-Cov Spike glycoprotein gene |
| PMV_00096 | ATGCACCGGCCACGGTTTGTG | SARS-Cov Spike glycoprotein gene |
| PMV_00097 | ATGCGCCAAGCTGGTGTGAGTTGA | SARS-Cov Spike glycoprotein gene |
| PMV_00098 | TGCTGGCGCTGCTCTTCAAATACC | SARS-Cov Spike glycoprotein gene |
| PMV_00099 | CGGGGCTGCTTGTGGGAAGG | SARS-Cov Spike glycoprotein gene |

In yet another example, the PCR is conducted using at least one of the following pairs of primers for a non-SARS-CoV infectious organism causing SARS-like symptoms set forth in Table 19.

TABLE 19
Exemplary primers for non-SARS-CoV infectious organism causing SARS-like symptoms

| | | |
|---|---|---|
| Id | Sequence (5′-3′) | species |

| | | |
|---|---|---|
| PMIA_00001 | TTTGTGCGACAATGCTTCA | Influenza A virus |
| PMIA_00002 | GACATTTGAGAAAGCTTGCC | Influenza A virus |
| PMIA_00003 | AGGGACAACCTNGAACCTGG | Influenza A virus |
| PMIA_00004 | AGGAGTTGAACCAAGACGCATT | Influenza A virus |
| PMIA_00005 | ACCACATTCCCTTATACTGGAG | Influenza A virus |
| PMIA_00006 | TTAGTCATCATCTTTCTCACAACA | Influenza A virus |
| PMIA_00007 | ACAAATTGCTTCAAATGAGAAC | Influenza A virus |
| PMIA_00008 | TGTCTCCGAAGAAATAAGATCC | Influenza A virus |
| PMIA_00009 | GCGCAGAGACTTGAAGATGT | Influenza A virus |
| PMIA_00010 | CCTTCCGTAGAAGGCCCT | Influenza A virus |
| PMIB_00001 | CACAATGGCAGAATTTAGTGA | Influenza B virus |
| PMIB_00002 | GTCAGTTTGATCCCGTAGTG | Influenza B virus |
| PMIB_00003 | CAGATCCCAGAGTGGACTCA | Influenza B virus |
| PMIB_00004 | TGTATTACCCAAGGGTTGTTAC | Influenza B virus |
| PMIB_00005 | GATCAGCATGACAGTAACAGGA | Influenza B virus |
| PMIB_00006 | ATGTTCGGTAAAAGTCGTTTAT | Influenza B virus |
| PMIB_00007 | CCACAGGGGAGATTCCAAAG | Influenza B virus |
| PMIB_00008 | GACATTCTTCCTGATTCATAATC | Influenza B virus |
| PMIB_00009 | CAAACAACGGTAGACCAATATA | Influenza B virus |
| PMIB_00010 | AGGTTCAGTATCTATCACAGTCTT | Influenza B virus |
| PMIB_00011 | ATGTCCAACATGGATATTGAC | Influenza B virus |
| PMIB_00012 | GCTCTTCCTATAAATCGAATG | Influenza B virus |
| PMIB_00013 | TGATCAAGTGATCGGAAGTAG | Influenza B virus |
| PMIB_00014 | GATGGTCTGCTTAATTGGAA | Influenza B virus |
| PMIB_00015 | ACAGAAGATGGAGAAGGCAA | Influenza B virus |
| PMIB_00016 | ATTGTTTCTTTGGCCTGGAT | Influenza B virus |
| PMAd1_00001 | TGGCGGTATAGGGGTAACTG | Human adenovirus |
| PMAd1_00002 | ATTGCGGTGATGGTTAAAGG | Human adenovirus |
| PMAd1_00003 | TTTTGCCGATCCCACTTATC | Human adenovirus |
| PMAd1_00004 | GCAAGTCTACCACGGCATTT | Human adenovirus |
| PMAd2_00001 | CTCCGTTATCGCTCCATGTT | Human adenovirus |
| PMAd2_00002 | AAGGACTGGTCGTTGGTGTC | Human adenovirus |
| PMAd2_00003 | AAATGCCGTGGTAGATTTGC | Human adenovirus |
| PMAd2_00004 | GTTGAAGGGGTTGACGTTGT | Human adenovirus |
| PMAd3_00001 | TTCCTCTGGATGGCATAGGAC | Human adenovirus |
| PMAd3_00002 | TGUGGTGTTAGTGGGCAAA | Human adenovirus |
| PMAd3_00003 | ACATGGTCCTGCAAAGTTCC | Human adenovirus |
| PMAd3_00004 | GCATTGTGCCACGTTGTATC | Human adenovirus |
| PMAd4_00001 | CGCTTCGGAGTACCTCAGTC | Human adenovirus |
| PMAd4_00002 | CTGCATCATTGGTGTCAACC | Human adenovirus |
| PMAd4_00003 | GGCACCTTTTACCTCAACCA | Human adenovirus |
| PMAd4_00004 | TCTGGACCAAGAACCAGTCC | Human adenovirus |
| PMAd5_00001 | GGCCTACCCTGCTAACTTCC | Human adenovirus |
| PMAd5_00002 | ATAAAGAAGGGTGGGCTCGT | Human adenovirus |
| PMAd5_00003 | ATCGCAGTTGAATGCTGTTG | Human adenovirus |
| PMAd5_00004 | GTTGAAGGGGTTGACGTTGT | Human adenovirus |
| PMAd7_00001 | ACATGGTCCTGCAAAGTTCC | Human adenovirus |
| PMAd7_00002 | GATCGAACCCTGATCCAAGA | Human adenovirus |
| PMAd7_00003 | AACACCAACCGAAGGAGATG | Human adenovirus |
| PMAd7_00004 | CCTATGCCATCCAGAGGAAA | Human adenovims |
| PMAd11_00001 | CAGATGCTCGCCAACTACAA | Human adenovirus |
| PMAd11_00002 | AGCCATGTAACCCACAAAGC | Human adenovirus |
| PMAd11_00003 | ACGGACGTTATGTGCCTTTC | Human adenovirus |
| PMAd11_00004 | GGGAATATTGGTTGCATTGG | Human adenovirus |
| PMAd21_00001 | ACTGGTTCCTGGTCCAGATG | Human adenovirus |
| PMAd21_00002 | AGCCATGTAACCCACAAAGC | Human adenovirus |
| PMAd21_00003 | CTGGATATGGCCAGCACTTT | Human adenovirus |
| PMAd21_00004 | CACCTGAGGTTCTGGTTGGT | Human adenovirus |
| PMAd23_00001 | TAATGAAAAGGGCGGACAAG | Human adenovirus |
| PMAd23_00002 | GCCAATGTAGTTTGGCCTGT | Human adenovirus |
| PMAd23_00003 | AACTCCGCGGTAGACAGCTA | Human adenovirus |
| PMAd23_00004 | CGTAGGTGTTGGTGTTGGTG | Human adenovirus |
| PMV_a0061 | TCACTTGCTTCCGTTGAGGUGGGGTGATGGGTTTCAGATTAA | HCoV-OC43 |
| PMV_a0062 | GGTTTCGGATGTTACAGCGTCTCGGGAAGATCGCCTTCTTCTA | HCoV-OC43 |
| PMV_b0061 | TTGGGGTGATGGGTTTCAGATTAA | HCoV-OC43 |
| PMV_b0062 | CTCGGGAAGATCGCCTTCTTCTA | HCoV-OC43 |
| PMV_a0053 | TCACTTGCTTCCGTTGAGGTTGGGCTGGCGGTTTAGAGTTGA | HCoV-229E |
| PMV_a0054 | GGTTTCGGATGTTACAGCGTGTGCGACCGCCCTTGTTTATGG | HCoV-229E |
| PMV_a0055 | TCACTTGCTTCCGTTGAGGGCGTTGTTGGCCTTTTTCTTGTCT | HCoV-229E |
| PMV_a0056 | GGTTTCGGATGTTACAGCGTGCCCGGCATTATTTCATTGTTCTG | HCoV-229E |
| PMV_a0057 | TCACTTGCTTCCGTTGAGGACAAAAGCCGCTGGTGGTAAAG | HCoV-229E |
| PMV_a0058 | GGTTTCGGATGTTACAGCGTCAGAAATCATAACGGGCAAACTCA | HCoV-229E |
| PMV_a0059 | TCACTTGCTTCCGTTGAGGAAGAGTTATTGCTGGCGTTGTTGG | HCoV-229E |
| PMV_a0060 | GGTTTCGGATGTTACAGCGTGCCCGGCATTATTTCATTGTTCTG | HCoV-229E |
| PMV_b0053 | TTGGGCTGGCGGTTTAGAGTTGA | HCoV-229E |
| PMV_b0054 | GTGCGACCGCCCTTGTTTATGG | HCoV-229E |
| PMV_b0055 | GCGTTGTTGGCCTTTTTCTTGTCT | HCoV-229E |
| PMV_b0056 | GCCCGGCATTATTTCATTGTTCTG | HCoV-229E |
| PMV_b0057 | ACAAAAGCCGCTGGTGGTAAAG | HCoV-229E |
| PMV_b0058 | CAGAAATCATAACGGGCAAACTCA | HCoV-229E |
| PMV_b0059 | AAGAGTTATTGCTGGCGTTGTTGG | HCoV-229E |
| PMV_b0060 | GCCCGGCATTATTTCATTGTTCTG | HCoV-229E |
| PMHE_00001 | GGTGGTAACCCCTCGCAGGA | Human enteric coronaviruse |
| PMHE_00002 | TGGCTCTTCCCTTTGGGCACT | Human enteric coronaviruse |
| PMHE_00003 | GAGAATGAACCTTATGTCGGCACCTG | Human enteric coronaviruse |
| PMHE_00004 | TTCCGCAAGTCTTTCACTTTCTCCAA | Human enteric coronaviruse |
| PMHE_00005 | CAGCTTTCAGCCAGGGACGTGT | Human enteric coronaviruse |
| PMHE_00006 | TTTCCAGCTTTTGCGCAGTGGT | Human enteric coronaviruse |
| PMHE_00007 | TCTGTTTTGGTGCAGGTCAATTTGTG | Human enteric coronaviruse |
| PMHE_00008 | ATGAACCAGGTCGTAAGCATCCTCAA | Human enteric coronaviruse |
| PMHE_00009 | GTTGCTTGTCAACCCCCGTACTGTTA | Human enteric coronaviruse |
| PMHE_00010 | AGGACACCTGCCATAGGGGTAGAGAG | Human enteric coronaviruse |
| PMHE_00011 | GGTTGTTGACTCGCGGTGGA | Human enteric coronaviruse |
| PMHE_00012 | GGGGTAGAGAGGCCAAACACTGC | Human enteric coronaviruse |
| PMRh_00001 | ACATGGTCCCATTGGATTGT | Human rhinovirus |
| PMRh_00002 | TGAGGAAATCTTTCGCCACT | Human rhinovirus |
| PMRh_00003 | ATGTTGCCCCCTAGTCTGTG | Human rhinovirus |
| PMRh_00004 | TTCTGAAGGTGGTGTGTTGC | Human rhinovirus |
| PMRh_00005 | TGGTATTCATGTTGGCGGTA | Human rhinovirus |
| PMRh_00006 | ACAGCAGGTTCCTTGTCACC | Human rhinovirus |
| PMRh_00007 | TCTTGCCTCCAATGGCTAGT | Human rhinovirus |
| PMRh_00008 | TGACATGCCTGCATTGAGTT | Human rhinovirus |
| PMRh_00009 | TCCCAATATGCCCTCTTCAG | Human rhinovirus |
| PMRh_00010 | CGCTGATGGGGATTGAGTAT | Human rhinovirus |
| PMRh_00011 | TGTGCTCAGTGTGCTTCCTC | Human rhinovirus |
| PMRh_00012 | TGCACCCATGATGACAATCT | Human rhinovirus |
| PMRh_00013 | GCAGTTCTTGCCAAAGAAGG | Human rhinovirus |
| PMRh_00014 | TGAAGGGTTTTTGGTCCATC | Human rhinovirus |
| PMRh_00015 | TGCCTGATGCCCTTAAAAAC | Human rhinovirus |
| PMRh_00016 | GGGTGTGATTGTACCCGACT | Human rhinovirus |
| PMMP_00001 | CTTAACAGTTGTATGCATTGGAAACT | Mycoplasma pneumoniae |
| PMMP_00002 | GTTTACGGTGTGGACTACTAGGGTAT | Mycoplasma pneumoniae |
| PMMP_00003 | CTATGCTGAGAAGTAGAATAGCCACA | Mycoplasma pneumoniae |
| PMMP_00004 | TGGTACAGTCAAACTCTAGCCATTAC | Mycoptasma pneumoniae |
| PMMP_00005 | ATACCCTAGTAGTCCACACCGTAAAC | Mycoplasma pneumoniae |
| PMMP_00006 | ATGTCAAGTCTAGGTAAGGTTTTTCG | Mycoplasma pneumoniae |
| PMMP_00007 | AGGCGAAAACTTAGGCCATT | Mycoplasma pneumoniae |
| PMMP_00008 | CCGTCAATTCCGTTTGAGTT | Mycoplasma pneumoniae |
| PMMP_00009 | CGACGGTACACGAAAAACCT | Mycoplasma pneumoniae |
| PMMP_00010 | TCCCTTCCTTCCTCCAATTT | Mycoplasma pneumoniae |
| PMR_00001 | ATTCCCATGGAGAAACTCCTAGAT | Rubella virus |
| PMR_00002 | GTGATCACTGACCTGCATCTG | Rubella virus |
| PMR_00003 | GTAAGAGACCACGTCCGATCAAT | Rubella virus |
| PMR_00004 | GAGGACGTGTAGGGCTTCTTTAG | Rubella virus |
| PMR_00005 | ATCGGACCTCGCTTAGGACT | Rubella virus |
| PMR_00006 | CTGGGTATCACGGCTACGAT | Rubella virus |
| PMR_00007 | AGAGACCACGTCCGATCAAT | Rubella virus |
| PMR_00008 | TGAGGACGTGTAGGGCTTCT | Rubella virus |
| PMR_00009 | GTCAACGCCTACTCCTCTGG | Rubella virus |
| PMR_00010 | GTCTTGTGAGGGTGCTGGAC | Rubella virus |
| PMM_00001 | CACATTGGCATCTGAACTCG | Measles virus |
| PMM_00002 | TCTGTTTGACCCTCCTGTCC | Measles virus |
| PMM_00003 | AGATTGCAATGCATACTACTGAGGAC | Measles virus |
| PMM_00004 | ATGCAGTGTCAATGTCTAGAGGTGT | Measles vIrus |
| PMM_00005 | CAATGCATACTACTGAGGACAGGA | Measles virus |
| PMM_00006 | ATGCAGTGTCAATGTCTAGAGGTG | Measles virus |
| PMM_00007 | TACCATCAGAGGTCAATTCTCAAA | Measles virus |
| PMM_00008 | CTACTTCAAACACTCGGTACATGC | Measles virus |
| PMM_00009 | CATGTCGCTGTCTCTGTTAGACTT | Measles virus |
| PMM_00010 | CAAGCCTGGATTTCTTATAACACC | Measles virus |
| PMRSV_00001 | AAACCAAAGAAGAAACCAACCAT | Human respiratory syncytial virus |
| PMRSV_00002 | TGTTCTAATGTGGTTGTGTCGAG | Human respiratory syncytial virus |
| PMRSV_00003 | TGCTAAAAGAGATGGGAGAAGTG | Human respiratory syncytial virus |
| PMRSV_00004 | ATCCTTTGGTATGAGACCCTTGT | Human respiratory syncytial virus |
| PMRSV_00005 | ACAAGGGTCTCATACCAAAGGAT | Human respiratory syncytial virus |
| PMRSV_00006 | GCTAAAACTCCCCATCTTAGCAT | Human respiratory syncytial virus |
| PMRSV_00007 | TTTATGATGCAGCCAAAGCA | Human respiratory syncytial virus |
| PMRSV_00008 | TCCATGAAATTCAGGTGCAA | Human respiratory syncytial virus |
| PMRSV_00009 | AAAAACACCAGCCAAAACGA | Human respiratory syncytial virus |
| PMRSV_00010 | CTGTGGGTGTTTGTGTGGAG | Human respiratory syncytial virus |
| PMRSV_00011 | CCAAAGCATATGCAGAGCAA | Human respiratory syncytial virus |
| PMRSV_00012 | TCCATGAAATTCAGGTGCAA | Human respiratory syncytial virus |
| PMPI_00001 | GCATGGAAACTAGCAGCACA | Parainfluenza |
| PMPI_00002 | GGTGTTGTGGTCTTCGAGGT | Parainfluenza |
| PMPI_00003 | GGCTCCATAGTATCATCGACAAC | Parainfluenza |
| PMPI_00004 | CCTAGAGGCCCTGTGTATACCTT | Parainfluenza |
| PMPI_00005 | ACACAACAAACAATGCAAACAAC | Parainfluenza |
| PMPI_00006 | TTAACATGCGCTTAGCAAATACA | Parainfluenza |
| PMPI_00007 | TTAGCTCACTCATTGGACACAGA | Parainfluenza |
| PMPI_00008 | GTCTCTCGTTTTGACAATGAACC | Parainfluenza |
| PMPI_00009 | TCTCACTACAAACGGTGTCAATG | Parainfluenza |
| PMPI_00010 | TCTAGATCCGCATTCTCTCTTTG | Parainfluenza |
| PMPI_00011 | ACAGATGGGTTCATTGTCAAAAC | Parainfluenza |
| PMPI_00012 | GCTTTGACCAACACTATCCAAAC | Parainfluenza |
| PMPI_00013 | GCTGAACACCCAGATTTACAAAG | Parainfluenza |
| PMPI_00014 | ACAGCTCTCCATTTCATGGTTTA | Parainfluenza |
| PMPI_00015 | ATATGCATTTGTCAATGGAGGAG | Parainfluenza |
| PMPI_00016 | CATTTGGTGTGTAAAATGCAAGA | Parainfluenza |
| PMPI_00017 | CACAGAACACCAGAACAACAAGA | Parainfluenza |
| PMPI_00018 | TTGGGACTGTTAACCAATACACC | Parainfluenza |
| PMME_00001 | CATCCCAAAAATTGCCAGAT | Human metapneumovirus |
| PMME_00002 | TTTGGGCTTTGCCTTAAATG | Human metapneumovirus |
| PMME_00003 | ACACCCTCATCATTGCAACA | Human metapneumovirua |
| PMME_00004 | GCCCTTCTGACTGTGGTCTC | Human metapneumovirus |
| PMME_00005 | CGACACAGCAGCAGGAATTA | Human metapneumovirus |
| PMME_00006 | TCAAAGCTGCTTGACACTGG | Human metapneumovirus |

In yet another example, the PCR is conducted using at least one of the following pairs of primers for a non-SARS-CoV infectious organism damaging the subject's immune system set forth in Table 20.

TABLE 20

Exemplary primers for non-SARS-CoV infectious
organism damaging the subject's immune system

| | | |
|---|---|---|
| id | sequence (5′-3′) | species |

| | | |
|---|---|---|
| PMTTV_00001 | TGGGGCCAGACTTCGCCATA | TTV |
| PMTTV_00002 | AGCTTCCGCCGAGGATGACC | TTV |
| PMTTV_00003 | CTTGGGGGCTCAACGCCTTC | TTV |
| PMTTV_00004 | GCGAAGTCTGGCCCCACTCA | TTV |
| PMTTV_00005 | CCACAGGCCAACCGAATGCT | TTV |
| PMTTV_00006 | AGCCCGAATTGCCCCTTGAC | TTV |
| PMTTV_00007 | AGCGAATCCTGGGAGTCAAACTCAG | TTV |
| PMTTV_00008 | GGCCTCGTACTCCTCTTTCCAGTCA | TTV |
| PMTTV_00009 | GCCCCTTTGCATACCACTCAGACAT | TTV |
| PMTTV_00010 | TGGAATGTGAGTTCCGGTGAGTTGT | TTV |
| PMTTV_00011 | TGTCAGTAACAGGGGTCGCCATAGA | TTV |
| PMTTV_00012 | TGTGACGTATGGACGACCTTTGACC | TTV |
| PMV_11047 | CACAGACAGAGGAGAAGGCAAC | TTV |
| PMV_11048 | AATAGGCACATTACTACTACCTCCTG | TTV |
| PMTP_00001 | GCGGTCGGTAGGAGGATAAAGGAAA | TP |
| PMTP_00002 | CCGGGGATTTGTCTACAGGGTTTCT | TP |
| PMTP_00003 | CAGACGCTCATCCAACTCCTGAGAA | TP |
| PMTP_00004 | CCGTTGTACCGTCTTTTGGACGTT | TP |
| PMTP_00005 | CACGCTCTACCTCATTCGAGAGCAA | TP |
| PMTP_00006 | GTTGTGTTGCAACGAACACGCTACA | TP |
| PMTP_00007 | AGCGGTCGGTAGGAGGATAAAGGAA | TP |
| PMTP_00008 | ACCGGGGATTGTCTACAGGGTTTC | TP |
| PMV_11025 | AACACGATCCGCTACGACTACTAC | TP |
| PMV_11026 | CCCTATACCCGTTCGCAATCAAAG | TP |
| PMHIV1_00001 | ATGGGCGCAGCCTCAATGAC | HIV1 |
| PMHIV1_00002 | CCCCAAATCCCCAGGAGCTG | HIV1 |
| PMHIV1_00003 | GGGACAGCTACAACCATCCCTTCAG | HIV1 |
| PMHIV1_00004 | GACCTGATTGCTGTGTCCTGTGTCA | HIV1 |
| PMHIV1_00005 | GGGATGGAAAGGATCACCAGCAATA | HIV1 |
| PMHIV1_00006 | GTCTGGTGTGGTAAGTCCCGACCTC | HIV1 |
| PMHIV1_00007 | AAGGATCAACAGCTCCTGGGGATTT | HIV1 |
| PMHIV1_00008 | TTCTTGCTGGTTTTGCGATTCTTCA | HIV1 |
| PMV_11055 | TAATCCACCTATCCCAGTAGGAGAAAT | HIV1 |
| PMV_11056 | GGTCCTTGTCTTATGTCCAGAATGC | HIV1 |
| PMV_11057 | TGGGAAGTTCAATTAGGAATACCAC | HIV1 |
| PMV_11058 | TCCTACATACAAATCATCCATGTATTG | HIV1 |
| PMHGV_00001 | GCCGTCGATGACTGCTTGAT | HGV |
| PMHGV_00002 | TCCGGAAGTCCGTGGTCAGG | HGV |
| PMHGV_00003 | ACGGTGGGAGTCGCGTTGAC | HGV |
| PMHGV_00004 | GGCCACGCAAACCAACAAGG | HGV |
| PMHGV_00005 | CGGCCAAAAGGTGGTGGATG | HGV |
| PMHGV_00006 | CGGGCTCGGTTTTAACGACGA | HGV |
| PMHGV_00007 | GCCACGGGCAAAATCAGTGG | HGV |
| PMHGV_00008 | TGTCGCGATCCGATGATCCA | HGV |
| PMHGV_00009 | CGCGTGTGAGCTAAAGTGGGAAAGT | HGV |
| PMHGV_00010 | ATCGTCACCAACAGGAAGACCATGA | HGV |
| PMHGV_00011 | TCGCTCTCGGGTTGGTTTTGTATTC | HGV |
| PMHGV_00012 | CATCCACCTTAGGCTCCCTGTTGAC | HGV |
| PMV_11045 | GGGTTGGTAGGTCGTAAATCCC | HGV |
| PMV_11046 | GTACGTGGGCGTCGTTTGC | HGV |
| PMV_11001 | CCTTTCCACCATCCAGCAGT | HEV |
| PMV_11002 | CGAGCTTTACCCACCTTCAGC | HEV |
| PMHEV_00001 | CTGGCGGTGGGCTCTGTCAT | HEV |
| PMHEV_00002 | ACCGAGGCGGGAGCAAGTCT | HEV |
| PMHEV_00003 | ACGGGCGGATCGATTGTGAG | HEV |
| PMHEV_00004 | GGCAGCGACATAGCGCACCT | HEV |
| PMHEV_00005 | AGCTCACCACCACGGCTGCT | HEV |
| PMHEV_00006 | CTGAGACGACGGGGCGAGAG | HEV |
| PMHEV_00007 | ATCGCGCCCCTTTTCTGTCC | HEV |
| PMHEV_00008 | GGGGGCGACCATCAAGTGTG | HEV |
| PMHDV_00001 | GACGGGCCGGCTGTTCTTCT | HDV |
| PMHDV_00002 | GACTCCGGGCCTGGGAAGAG | HDV |
| PMHDV_00003 | ACTCCGGCCGAAAGGTCGAG | HDV |
| PMHDV_00004 | GGCGGAACACCCACCGACTA | HDV |
| PMHDV_00005 | CCATGACTCTGGAGACATCCTGGAA | HDV |
| PMHDV_00006 | CGTCAGAGCTCTCTGTTCGCTGAAG | HDV |
| PMHDV_00007 | CCTTCTCTCGTCTTCCTCGGTCAAC | HDV |
| PMHDV_00008 | CCGAACGGACCAGATGGAGATAGAC | HDV |
| PMHDV_00009 | GCTCCCGAGAGGGATAAAACGGTAA | HDV |
| PMHDV_00010 | GAGTGCTCTCCAAACTTGGCAGTTG | HDV |
| PMHDV_00011 | TCTCGTCTTCCTTCGGTCAACCTCTT | HDV |
| PMHDV_00012 | CCGAACGGACCAGATGGAGATAGAC | HDV |
| PMV_11041 | AACATTCCGAAGGGGACCGT | HDV |
| PMV_11042 | GGCATCCGAAGGAGGACG | HDV |
| PMHCV_00001 | GGCGCTGGAAAGAGGGTCTACTACC | HCV |
| PMHCV_00002 | TGTTCAAGCTGATCCCTGGCTATGA | HCV |
| PMHCV_00003 | ACATCTGGGACTGGATATGCGAGGT | HCV |
| PMHCV_00004 | ATCCTCATCGTCCCGTTTTTGACAT | HCV |
| PMHCV_00005 | TGTGCCAGGACCATCTTGAATTTTG | HCV |
| PMHCV_00006 | AGGCGGATCAAACACTTCCACATCT | HCV |
| PMHCV_00007 | GGGGTGCAAATGATACGGATGTCTT | HCV |
| PMHCV_00008 | AGAGTATGTGGCTTCCGGATGCTTG | HCV |
| PMHCV_00009 | ACACGCCGTGGGCCTATTCA | HCV |
| PMHCV_00010 | GCCGGGACCTTGGTGCTCTT | HCV |
| PMHCV_00011 | CACGCCGTGGGCCTATTCAG | HCV |
| PMHCV_00012 | GCCGGGACCTTGGTGCTCTT | HCV |
| PMV_11039 | CTCGCAAGCACCCTATCAGGCAGT | HCV |
| PMV_11040 | GCAGAAAGCGTCTAGCCATGGCGT | HCV |
| PMHCMV_00001 | GCGCCTGCTGCTCGAAATGT | HCMV |
| PMHCMV_00002 | GTCGCGGCTGTTGCGGTAGT | HCMV |
| PMHCMV_00003 | CCCCACGTCCATCTGCGTCT | HCMV |
| PMHCMV_00004 | GCCCCCAGCAGTCTCACCAG | HCMV |
| PMHCMV_00005 | GCTCACGCACCCTGGAGGAC | HCMV |
| PMHCMV_00006 | AGTTCCAGCCCACGCACCAG | HCMV |
| PMHCMV_00007 | GTGCAGTTTAGGTGGCAGTTCATGC | HCMV |
| PMHCMV_00008 | GGAAAGGGGAGGGTAGAAACGTGAG | HCMV |
| PMHCMV_00009 | TGTGATTGCGTGTGCAGTTTAGGTG | HCMV |
| PMHCMV_00010 | GGGGAGGGTAGAAACGTGAGTCTCC | HCMV |
| PMV_11051 | ATTCCAAGCGGCCTCTGATAA | HCMV |
| PMV_11052 | TCTTCCTCTGGGGCAACTTCC | HCMV |
| PMHBV_00001 | TCGCAGTCCCCAACCTCCAA | HBV |
| PMHBV_00002 | CAGGGTCCCGTGCTGGTTGT | HBV |
| PMHBV_00003 | GCAGCCGGTCTGGAGCAAAA | HBV |
| PMHBV_00004 | GCAGACGGAGAAGGGGACGA | HBV |
| PMHBV_00005 | CGCCTCATTTTGCGGGTCAC | HBV |
| PMHBV_00006 | TGGTTGGCTTGTGGCCAGTG | HBV |
| PMHBV_00007 | ATCAAGGTATGTTGCCCGTTTGTCC | HBV |
| PMHBV_00008 | AGGCCCACTCCCATAGGTATTTTGC | HBV |
| PMHBV_00009 | CCTAGGACCCCTGCTCGTGTTACAG | HBV |
| PMHBV_00010 | GCGATAACCAGGACAAATTGGAGGA | HBV |
| PMHBV_00011 | CTGCGCACCATTATCATGCAACTTT | HBV |
| PMHBV_00012 | AGTAGATCCCGGACGGAAGGAAAGA | HBV |
| PMV_11037 | GTTCAAGCCTCCAAGCTGTG | HBV |
| PMV_11038 | TCAGAAGGCAAAAAAGAGAGTAACT | HBV |
| PMHAV_00001 | GATGTTTGGGACGTCACCTT | HAV |
| PMHAV_00002 | CTGGATGAGAGCCAGTCCTC | HAV |
| PMHAV_00003 | ATTGCATTGGCAACCAAAAT | HAV |
| PMHAV_00004 | ATCTCATTGGGCATCCTGAC | HAV |
| PMHAV_00005 | GACTGGAGGTTGGGAAACAA | HAV |
| PMHAV_00006 | AGCAGCCAGAGAGAATCCAA | HAV |
| PMHAV_00007 | TAAGCATTTTTCCCGCAAAG | HAV |
| PMHAV_00008 | AGGCATTCATGACCCATCTC | HAV |
| PMHAV_00009 | CCAACCAAATATCATTCAGGTAGAC | HAV |
| PMHAV_00010 | GACTTCGTGTACCTATTCACTCGAT | HAV |
| PMHAV_00011 | GGGTTTCCTTATGTTCAAGAAAAAT | HAV |
| PMHAV_00012 | CCAAAACTTTCTCTAATGGTCTCAA | HAV |
| PMV_11035 | TTTTGCTCCTCTTTACCATGCTATG | HAV |
| PMV_11036 | GGAAATGTCTCAGGTACTTTCTTTG | HAV |
| PMEBV_00001 | AACCCAATAGCATGACAGCCAATCC | EBV |
| PMEBV_00002 | TCAGCCCCAGAGACACGGTATATGA | EBV |
| PMEBV_00003 | TGAACCTGGGACCTATTGATGCAGA | EBV |
| PMEBV_00004 | CAGGGGAATCTCTGCCAACTTTGAG | EBV |
| PMEBV_00005 | TGCACAGTGACAGTGGGAGAAACAC | EBV |
| PMEBV_00006 | AAGAATGGAAAGGGTTGGCAGTGTG | EBV |
| PMEBV_00007 | GTGCACAGTGACAGTGGGAGAAACA | EBV |
| PMEBV_00008 | AAGAATGGAAAGGGTTGGCAGTGTG | EBV |
| PMV_11053 | CCCACGCGCGCATAATG | EBV |
| PMV_11054 | TTCACTTCGGTCTCCCCTAG | EBV |
| PMB19_00001 | TGGGCCGCCAAGTACAGGAA | B19 |
| PMB19_00002 | GGGTTGCCCGCcTAAAATGG | B19 |
| PMB19_00003 | CCCTATTAGTGGGGCAGCATGTGTT | B19 |
| PMB19_00004 | CCACCAAGCTTTTCCCTGCTACATC | B19 |
| PMB19_00005 | CAGTGTCACAGCCATACCACCACTG | B19 |
| PMB19_00006 | TGCTGGGTTCCTTTATTGGGGAAAT | B19 |
| PMB19_00007 | CCCATTGCATTAATGTAGGGGCTTG | B19 |
| PMB19_00008 | ATCACTTTCCCACCATTTGCCACTT | B19 |
| PMV_11049 | CCTTTCCACCATCCAGCAGT | B19 |
| PMV_11050 | CGAGCTTTACCCACCTTCAGC | B19 |

In yet another example, the PCR is conducted using at least one of the following pairs of primers for a non-SARS-CoV coronaviridae virus set forth in Table 21.

TABLE 21

Exemplary primers for non-SARS-CoV
coronaviridae virus

| | |
|---|---|
| seqid | sequence (5′-3′) |

| | |
|---|---|
| PMIBV_00001 | GGAACAGGACCTGCCGCTGA |
| PMIBV_00002 | ATCAGGTCCGCCATCCGAGA |
| PMIBV_00003 | AAAGGTGGAAGAAAACCAGTCCCAGA |
| PMIBV_00004 | GCCATCCGAGAATCGTAGTGGGTATT |
| PMMHV_00001 | CAGCGCCAGCCTGCCTCTAC |
| PMMHV_00002 | TGCTGCACTGGGCACTGCTT |
| PMMHV_00003 | GGAAATTACCGACTGCCCTCAAACA |
| PMMHV_00004 | TGATTATTTGGTCCACGCTCGGTTT |
| PMEQ_00001 | TCCCGCGCATCCAGTAGAGC |
| PMEQ_00002 | CTGCGGCTTTGTGGCATCCT |
| PMEQ_00003 | TTTGCTGAAGGACAAGGTGTGCCTA |
| PMEQ_00004 | CCAGAAGACTCCGTCAATGTTGGTG |
| PMCA_00001 | AAAAACGTGGTCGTTCCAATTCTCG |
| PMCA_00002 | CCATGCGATAGCGGCTTTGTCTATT |
| PMCA_00003 | TGGGAACGGTGCCAAGCATT |
| PMCA_00004 | GCCACCTCTGATGGACGAGCA |
| PMFE_00001 | CGCGTCAACTGGGGAGATGAA |
| PMFE_00002 | GCGCGCCTGTCTGTTCCAAT |
| PMFE_00003 | GAGTCTTCTGGGTTGCAAAGGATGG |
| PMFE_00004 | CCCCTGGATTGAGACCTGTTTCTTG |
| PMPEDV_00001 | GCAGCATTGCTCTTTGGTGGTAATG |
| PMPEDV_00002 | TGCTGAATGGTTTCACGCTTGTTCT |
| PMPEDV_00003 | CCGCAAACGGGTGCCATTAT |
| PMPEDV_00004 | TCGCCGTGAGGTCCTGTTCC |
| PMPTGV_00001 | TCGCTCCAATTCCCGTGGTC |
| PMPTGV_00002 | ACGTTGGCCCTTCACCATGC |
| PMPTGV_00003 | CAAGCATTACCCACAATTGGCTGAA |
| PMPTGV_00004 | TTCTTTTGCCACTTCTGATGGACGA |
| PMBOV_00001 | TTCCTTTAAAACAGCCGATGGCAAC |
| PMBOV_00002 | TCGGAATAGCCTCATCGCTACTTGG |
| PMBOV_00003 | TTCCGCCTGGCACGGTACTC |
| PMBOV_00004 | TGGCTTAGCGGCATCCTTGC |
| PMFIPV_00001 | CACCATGGCCTCAGCCTTGA |
| PMFIPV_00002 | GTGCCGCCAACCTGCCAGTA |
| PMFIPV_00003 | GGTCTTGGCACTGTGGATGATGATT |
| PMFIPV_00004 | GAAAAAGGGACAGCTACAGCGGATG |
| PMR_00001 | CCCAATCAGAATTTTGGAGGCTCTG |
| PMR_00002 | AGCGAATTGCACCTGAATACTGCAA |
| PMR_00003 | TGACCAAACCGAGCGTGCAG |
| PMR_00004 | CAGTGGCGGGGATTCCATTG |
| PMPHEV_00001 | AGCGTCAACTGCTGCCACGA |
| PMPHEV_00002 | AGTACCGTGCCAGGCGGAAA |
| PMPHEV_00003 | AAGGTGTGCCTATTGCACCAGGAGT |
| PMPHEV_00004 | ACTAGCGACCCAGAAGACTCCGTCA |
| PMPV_00001 | AGAAGACCACTTGGGCTGACCAAAC |
| PMPV_00002 | TTGGCAATAGGCACTCCTTGTCCTT |
| PMPV_00003 | GCGCCAGCCTGCTTGTATTG |
| PMPV_00004 | TGGGGCCCCTCTTTCCAAAA |
| PMTK_00001 | ATGGCTCACCGCCGGTATTG |
| PMTK_00002 | TGGGCGTCACTCTGCTTCCA |
| PMTK_00003 | GCTAAGGCTGATGAAATGGCTCACC |
| PMTK_00004 | TCCAAAAAGACAAGCATGGCTGCTA |
| PMSDAV_00001 | TCTATGTTAAGGCTCGGGAAGGTC |
| PMSDAV_00002 | TACTTGCTTAGGCTGTCCGGCATCT |
| PMSDAV_00003 | AGCAGTGCCCAGTGCAGCAG |
| PMSDAV_00004 | TGGGTTCATCAACGCCACCA |

D. SARS-CoV and Non-SARS-CoV Infectious Organism Primers, Probes, Kit and Uses Thereof

In still another aspect, the present invention is directed to an oligonucleotide primer for amplifying a SARS-CoV and/or a non-SARS-CoV infectious organism nucleotide sequence, which oligonucleotide primer comprises a nucleotide sequence that: a) hybridizes, under high stringency, with a target SARS-CoV or a non-SARS-CoV infectious organism nucleotide sequence, or a complementary strand thereof, that is set forth in Table 18 or Tables 19-21; or b) has at least 90% identity to a target SARS-CoV or a non-SARS-CoV infectious organism nucleotide sequence comprising a nucleotide sequence, or a complementary strand thereof, that is set forth in Table 18 or Tables 19-21.

The present primers can comprise any suitable types of nucleic acids, e.g., DNA, 15 RNA, PNA or a derivative thereof. Preferably, the primers comprise a nucleotide sequence, or a complementary strand thereof, that is set forth in Table 18 or Tables 19-21.

In a specific embodiment, the present invention is directed to a kit for amplifying a SARS-CoV or a non-SARS-CoV infectious organism nucleotide sequence, which kit comprises: a) an above-described primer; and b) a nucleic acid polymerase that can amplify a SARS-CoV or a non-SARS-CoV infectious organism nucleotide sequence using the probe. Preferably, the nucleic acid polymerase is a reverse transcriptase.

In yet another aspect, the present invention is directed to an oligonucleotide probe for hybridizing to a SARS-CoV or a non-SARS-CoV infectious organismnucleotide sequence, which oligonucleotide probe comprises a nucleotide sequence that: a) hybridizes, under high stringency, with a target SARS-CoV or a non-SARS-CoV infectious organism nucleotide sequence, or a complementary strand thereof, that is set forth in Table 13 or Tables 15-17; or b) has at least 90% identity to a target SARS-CoV or a non-SARS-CoV infectious organism nucleotide sequence comprising a nucleotide sequence, or a complementary strand thereof, that is set forth in Table 13 or Tables 15-17.

The present probes can comprise any suitable types of nucleic acids, e.g., DNA, RNA, PNA or a derivative thereof. Preferably, the probes comprise a nucleotide sequence, or a complementary strand thereof, that is set forth in Table 13 or Tables 15-17. Also preferably, the probes are labeled, e.g., a chemical, an enzymatic, an immunogenic, a radioactive, a fluorescent, a luminescent and a FRET label.

In a specific embodiment, the present invention is directed to a kit for hybridization analysis of a SARS-CoV and/or a non-SARS-CoV infectious organism nucleotide sequence, which kit comprises: a) an above-described probe; and b) a means for assessing a hybrid formed between a SARS-CoV and/or a non-SARS-CoV infectious organism nucleotide sequence and said probe.

The oligonucleotide primers and probes can be produced by any suitable method. For example, the probes can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191(1981). Alternatively, automated synthesis may be preferred, for example, on a Applied Biosynthesis DNA synthesizer using cyanoethyl phosphoramidite chemistry. Preferably, the probes and the primers are chemically synthesized.

Suitable bases for preparing the oligonucleotide probes and primers of the present invention may be selected from naturally occurring nucleotide bases such as adenine, cytosine, guanine, uracil, and thymine. It may also be selected from nonnaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6 -methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N6-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3 ′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide probe should possess a sequence at least a portion of which is capable of binding to a portion of the sequence of a target SARS-CoV sequence.

Hybridization probes or amplification primers can be of any suitable length. There is no lower or upper limits to the length of the probe or primer, as long as the probe hybridizes to the SARS-CoV or the non-SARS-CoV infectious organism target nucleic acids and functions effectively as a probe or primer (e.g., facilitates detection or amplification). The probes and primers of the present invention can be as short as 50, 40, 30, 20, 15, or 10 nucleotides, or shorter. Likewise, the probes or primers can be as long as 20, 40, 50, 60, 75, 100 or 200 nucleotides, or longer, e.g., to the full length of the SARS-CoV or the non-SARS-CoV infectious organism target sequence. Generally, the probes will have at least 14 nucleotides, preferably at least 18 nucleotides, and more preferably at least 20 to 30 nucleotides of either of the complementary target nucleic acid strands and does not contain any hairpin secondary structures. In specific embodiments, the probe can have a length of at least 30 nucleotides or at least 50 nucleotides. If there is to be complete complementarity, i.e., if the strand contains a sequence identical to that of the probe, the duplex will be relatively stable under even stringent conditions and the probes may be short, i.e., in the range of about 10-30 base pairs. If some degree of mismatch is expected in the probe, i.e., if it is suspected that the probe would hybridize to a variant region, or to a group of sequences such as all species within a specific genus, the probe may be of greater length (i.e., 15-40 bases) to balance the effect of the mismatch(es).

The probe need not span the entire SARS-CoV or the non-SARS-CoV infectious organism target gene. Any subset of the target region that has the potential to specifically identify SARS-CoV or the non-SARS-CoV infectious organism target or alelle can be used. Consequently, the nucleic acid probe may hybridize to as few as 8 nucleotides of the target region. Further, fragments of the probes may be used so long as they are sufficiently characteristic of the SARS-CoV or the non-SARS-CoV infectious organism target gene to be typed.

The probe or primer should be able to hybridize with a SARS-CoV or a non-SARS-CoV infectious organism target nucleotide sequence that is at least 8 nucleotides in length under low stringency. Preferably, the probe or primer hybridizes with a SARS-CoV or a non-SARS-CoV infectious organism target nucleotide sequence under middle or high stringency.

In still another aspect, the present invention is directed to an array of oligonucleotide probes immobilized on a support for typing a SARS-CoV or a non-SARS-CoV infectious organism target gene, which array comprises a support suitable for use in nucleic acid hybridization having immobilized thereon a plurality of oligonucleotide probes, at least one of said probes comprising a nucleotide sequence that: a) hybridizes, under high stringency, with a target SARS-CoV or a non-SARS-CoV infectious organism nucleotide sequence, or a complementary strand thereof, that is set forth in Table 13 or Tables 15-17; or b) has at least 90% identity to a target SARS-CoV or a non-SARS-CoV infectious organism nucleotide sequence comprising a nucleotide sequence, or a complementary strand thereof, that is set forth in Table 13 or Tables 15-17.

The plurality of probes can comprise DNA, RNA, PNA or a derivative thereof. At least one or some of the probes can comprise a nucleotide sequence, or a complementary strand thereof, that is set forth in Table 13 or Tables 15-17. Preferably, probe arrays comprise all of the nucleotide sequences, or a complementary strand thereof, that are set forth in Table 13 or Tables 15-17. At least one, some or all of the probes can be labeled. Exemplary labels include a chemical, an enzymatic, an immunogenic, a radioactive, a fluorescent, a luminescent and a FRET label. Any suitable support, e.g., a silicon, a plastic, a glass, a ceramic, a rubber, and a polymer surface, can be used.

E. Assay Formats

Immobilization of Probes

The present methods, probes and probe arrays can be used in solution. Preferably, it is conducted in chip format, e.g., by using the probe(s) immobilized on a solid support.

The probes can be immobilized on any suitable surface, preferably, a solid support, such as silicon, plastic, glass, ceramic, rubber, or polymer surface. The probe may also be immobilized in a 3-dimensional porous gel substrate, e.g., Packard HydroGel chip (Broude et al., Nucleic Acids Res., 29(19):E92 (2001)).

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143,854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si—OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89(1989); Saiki et al., Proc. Natl. Acad. Sci. USA, 86:6230-6234(1989); and Gravitt et al., J Clin. Micro., 36:3020-3027(1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combinations thereof. Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

Hybridization Conditions

Hybridization can be carried out under any suitable technique known in the art. It will be apparent to those skilled in the art that hybridization conditions can be altered to increase or decrease the degree of hybridization, the level of specificity of the hybridization, and the background level of non-specific binding (i.e., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

Hybridization between the probe and target nucleic acids can be homogenous, e.g., typical conditions used in molecular beacons (Tyagi S. et al., Nature Biotechnology, 14:303-308 (1996); and U.S. Pat. No. 6,150,097 ) and in hybridization protection assay (Gen-Probe, Inc) (U.S. Pat. No. 6,004,745), or heterogeneous (typical conditions used in different type of nitrocellulose based hybridization and those used in magnetic bead based hybridization).

The target polynucleotide sequence may be detected by hybridization with an oligonucleotide probe that forms a stable hybrid with that of the target sequence under high to low stringency hybridization and wash conditions. An advantage of detection by hybridization is that, depending on the probes used, additional specificity is possible. If it is expected that the probes will be completely complementary (i.e., about 99% or greater) to the target sequence, high stringency conditions will be used. If some mismatching is expected, for example, if variant strains are expected with the result that the probe will not be completely complementary, the stringency of hybridization may be lessened. However, conditions are selected to minimize or eliminate nonspecific hybridization.

Conditions those affect hybridization and those select against nonspecific hybridization are known in the art (Molecular Cloning A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1×SSC, 0.1% SDS, at about 65° C. incubation/wash temperature. Middle stringent conditions are incubation in solutions that contain approximately 1-2×SSC, 0.1% SDS and about 50° C.-65° C. incubation/wash temperature. The low stringency conditions are 2×SSC and about 30° C.-50° C.

An alternate method of hybridization and washing is first to carry out a low stringency hybridization (5×SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad Sci. USA, 82:1585-1588 (1985)).

A hybridization solution may contain 25% formamide, 5×SSC, 5×Denhardt's solution, 100 μg/ml of single stranded DNA, 5% dextran sulfate, or other reagents known to be useful for probe hybridization.

Detection of the Hybrid

Detection of hybridization between the probe and the target SARS-CoV nucleic acids can be carried out by any method known in the art, e.g., labeling the probe, the secondary probe, the target nucleic acids or some combination thereof, and are suitable for purposes of the present invention. Alternatively, the hybrid may be detected by mass spectroscopy in the absence of detectable label (e.g., U.S. Pat. No. 6,300,076).

The detectable label is a moiety that can be detected either directly or indirectly after the hybridization. In other words, a detectable label has a measurable physical property (e.g., fluorescence or absorbance) or is participant in an enzyme reaction. Using direct labeling, the target nucleotide sequence or the probe is labeled, and the formation of the hybrid is assessed by detecting the label in the hybrid. Using indirect labeling, a secondary probe is labeled, and the formation of the hybrid is assessed by the detection of a secondary hybrid formed between the secondary probe and the original hybrid.

Methods of labeling probes or nucleic acids are well known in the art. Suitable labels include fluorophores, chromophores, luminophores, radioactive isotopes, electron dense reagents, FRET(fluorescence resonance energy transfer), enzymes and ligands having specific binding partners. Particularly useful labels are enzymatically active groups such as enzymes (Wisdom, Clin. Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No. 4,134,792); fluorescers (Soini and Hemmila, Clin. Chem., 25:353 (1979)); chromophores including phycobiliproteins, luminescers such as chemiluminescers and bioluminescers (Gorus and Schram, Clin Chem., 25:512 (1979) and ibid, 1531); specifically bindable ligands, i.e., protein binding ligands; antigens; and residues comprising radioisotopes such as ³H, ³⁵S, ³²P, ¹²⁵I, and ¹⁴C. Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (i.e., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe or nucleic acid may be labeled with a moiety that requires additional reagents to detect the hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Exemplary examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono-or bis-azido aminoalkyl methidium or ethidium compounds, ethidium monoazide ethidium diazide, ethidium dimer azide (Mitchell et al., J. Am. Chem. Soc., 104:4265 (1982))), 4-azido-7-chloroquinoline, 2-azidofluorene, 4′-aminomethyl4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylazides prefer insertion reactions over their rearrangement products (White et al., Meth. Enzymol., 46:644 (1977)).

The probe may also be modified for use in a specific format such as the addition of 10-100 T residues for reverse dot blot or the conjugation to bovine serum albumin or immobilization onto magnetic beads.

When detecting hybridization by an indirect detection method, a detectably labeled second probe(s) can be added after initial hybridization between the probe and the target or during hybridization of the probe and the target. Optionally, the hybridization conditions may be modified after addition of the secondary probe. After hybridization, unhybridized secondary probe can be separated from the initial probe, for example, by washing if the initial probe is immobilized on a solid support. In the case of a solid support, detection of label bound to locations on the support indicates hybridization of a target nucleotide sequence in the sample to the probe.

The detectably labeled secondary probe can be a specific probe. Alternatively, the detectably labeled probe can be a degenerate probe, e.g., a mixture of sequences such as whole genomic DNA essentially as described in U.S. Pat. No. 5,348,855. In the latter case, labeling can be accomplished with intercalating dyes if the secondary probe contains double stranded DNA. Preferred DNA-binding ligands are intercalator compounds such as those described above.

A secondary probe also can be a library of random nucleotide probe sequences. The length of a secondary probe should be decided in view of the length and composition of the primary probe or the target nucleotide sequence on the solid support that is to be detected by the secondary probe. Such a probe library is preferably provided with a 3′ or 5′ end labeled with photoactivatable reagent and the other end loaded with a detection reagent such as a fluorophore, enzyme, dye, luminophore, or other detectably known moiety.

The particular sequence used in making the labeled nucleic acid can be varied. Thus, for example, an amino-substituted psoralen can first be photochemically coupled with a nucleic acid, the product having pendant amino groups by which it can be coupled to the label, i.e., labeling is carried out by photochemically reacting a DNA binding ligand with the nucleic acid in the test sample. Alternatively, the psoralen can first be coupled to a label such as an enzyme and then to the nucleic acid.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinimidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaerythritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Secondary probe for indirect detection of hybridization can be also detected by energy transfer such as in the “beacon probe” method described by Tyagi and Kramer, Nature Biotech, 14:303-309 (1996) or U.S. Pat. Nos. 5,119,801 and 5,312,728 to Lizardi et al. Any FRET detection system known in the art can be used in the present method. For example, the AlphaScreen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include Fluorescein (donor) and tetramethylrhodamine (acceptor) with an effective distance of 55 Å; LAEDANS (donor) and Fluorescein (acceptor) with an effective distance of 46 Å; and Fluorescein (donor) and QSY-7 dye (acceptor) with an effective distance of 61 Å (Molecular Probes).

Quantitative assays for nucleic acid detection also can be performed according to the present invention. The amount of secondary probe bound to a microarray spot can be measured and can be related to the amount of nucleic acid target which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

Test Samples

Any suitable samples, including samples of human, animal, or environmental (e.g., soil or water) origin, can be analyzed using the present method. Test samples can include body fluids, such as urine, blood, semen, cerebrospinal fluid, pus, amniotic fluid, tears, or semisolid or fluid discharge, e.g., sputum, saliva, lung aspirate, vaginal or urethral discharge, stool or solid tissue samples, such as a biopsy or chorionic villi specimens. Test samples also include samples collected with swabs from the skin, genitalia, or throat.

Test samples can be processed to isolate nucleic acid by a variety of means well known in the art (See generally, Ausubel (Ed.) Current Protocols in Molecular Biology, 2. Preparation and Analysis of DNA and 4. Preparation and Analysis of RNA, John Wiley & Sons, Inc. (2000)). It will be apparent to those skilled in the art that target nucleic acids can be RNA or DNA that may be in form of direct sample or purified nucleic acid or amplicons.

Purified nucleic acids can be extracted from the aforementioned samples and may be measured spectraphotometrically or by other instrument for the purity. For those skilled in the art of nucleic acid amplification, amplicons are obtained as end products by various amplification methods such as PCR (Polymerase Chain Reaction, U.S. Pat. Nos. 4,683,195, 4,683,202, 4,800,159 and 4,965,188), NASBA (Nucleic Acid Sequence Based Amplification, U.S. Pat. No. 5,130,238), TMA (Transcription Mediated Amplification) (Kwoh et al., Proc. Natl. Acad Sci, USA, 86:1173-1177 (1989)), SDA (Strand Displacement Amplification, described by Walker et al., U.S. Pat. No. 5,270,184), tSDA (thermophilic Strand Displacement Amplification (U.S. Pat. No. 5,648,211 and Euro. Patent No. EP 0 684315), SSSR (Self-Sustained Sequence Replication) (U.S. Pat. No. 6,156,508).

In a specific embodiment, a sample of human origin is assayed. In yet another specific embodiment, a sputum, urine, blood, tissue section, food, soil or water sample is assayed.

Kits

The present probes can be packaged in a kit format, preferably with an instruction for using the probes to detect a target gene. The components of the kit are packaged together in a common container, typically including written instructions for performing selected specific embodiments of the methods disclosed herein. Components for detection methods, as described herein, may optionally be included in the kit, for example, a second probe, and/or reagents and means for carrying out label detection (e.g., radiolabel, enzyme substrates, antibodies, etc., and the like).

### F. EXAMPLES

### Example 1

### Probe Designs

Various genome sequences of SARS-CoV are available (See e.g., Table 22).

TABLE 22
Genome sequences of SARS coronaviruse currently obtained (as of
May 2, 2003)

| | | | | Number | | |
|---|---|---|---|---|---|---|
| | Source of | Submitting | | of N in | Length | |
| | SARS | Country | GenBank | the | of the | Percentage |
| ID | coronaviruse | (Area) | Acc | sequence | genome | of N |
| SARS_BJ01 | Beijing, | China | AY278488 | 900 | 28920 | 3.11% |
| | China | | | | | |
| SARS_BJ02 | Beijing, | China | AY278487 | 300 | 29430 | 1.02% |
| | China | | | | | |
| SARS_BJ03 | Beijing, | China | AY278490 | 607 | 29291 | 2.07% |
| | China | | | | | |
| SARS_GZ01 | Guangzhou, | China | AY278489 | 1007 | 29429 | 3.42% |
| | China | | | | | |
| SARS_BJ04 | Beijing, | China | AY279354 | 2502 | 24774 | 10.10% |
| | China | | | | | |
| SARS_CUHK- | Hong Kong, | Hong | AY278554 | 0 | 29736 | 0.00% |
| W1 | China | Kong, | | | | |
| | | China | | | | |
| SARS_HKU- | Hong Kong, | Hong | AY278491 | 0 | 29742 | 0.00% |
| 39849 | China | Kong, | | | | |
| | | China | | | | |
| SARS_Urbani | Vietnam | U.S. | AY278741 | 0 | 29727 | 0.00% |
| SARS_TOR2 | Toronto, | Canada | AY274119 | 0 | 29736 | 0.00% |
| | Canada | | | | | |

The sizes of the nine genomes shown in Table 22 are very similar. The five genomes submitted by China contain various levels of unidentified nucleotides (N).

The following Table 23 shows similarities or homologies among the nine 5 genomes of SARS coronaviruse.

TABLE 23
Comparison of similarities between the nine genomes of SARS
coronaviruse

| | BJ01 | BJ02 | BJ03 | GZ01 | BJ04 | CUHK-W1 | HKU-39849 | Urbani | TOR2 |
|---|---|---|---|---|---|---|---|---|---|
| BJ01 | | [symbol] | [symbol] | [symbol] | 91 | [symbol] | [symbol] | [symbol] | [symbol] |
| BJ02 | [symbol] | | [symbol] | 94 | 88 | [symbol] | [symbol] | [symbol] | [symbol] |
| BJ03 | [symbol] | [symbol] | | [symbol] | 89 | [symbol] | [symbol] | [symbol] | [symbol] |
| GZ01 | [symbol] | 94 | [symbol] | | 91 | [symbol] | [symbol] | [symbol] | [symbol] |
| BJ04 | 91 | 88 | 89 | 91 | | 89 | 89 | 89 | 89 |
| CUHK-W1 | [symbol] | [symbol] | [symbol] | [symbol] | 89 | | [symbol] | [symbol] | [symbol] |
| HKU-39849 | [symbol] | [symbol] | [symbol] | [symbol] | 89 | [symbol] | | [symbol] | [symbol] |
| Urbani | [symbol] | [symbol] | [symbol] | [symbol] | 89 | [symbol] | [symbol] | | [symbol] |
| TOR2 | [symbol] | [symbol] | [symbol] | [symbol] | 89 | [symbol] | [symbol] | [symbol] | |

The similarity of the nine genomes of SARS coronaviruse were compared. The numbers shown in the Table 23 represent the percentage of similarity between two genomes. Each number in Table 23 equals to the number of the same bases in two genomes divided by the total number of bases (about 30,000 bases) compared and then timed by 100.

Table 23 shows that the different genomes of SARS coronaviruse are highly similar to each other except BJ04. The similarity lower than 99% is caused by the presence of N in the nucleotide sequence. If all the Ns in the nucleotide sequences from BJ01-BJ04 and GZ01 are considered as the same with other genome (this assumption is reasonable based on comparison of other part of the genomes), the nine genomes are 99% similar to each other.

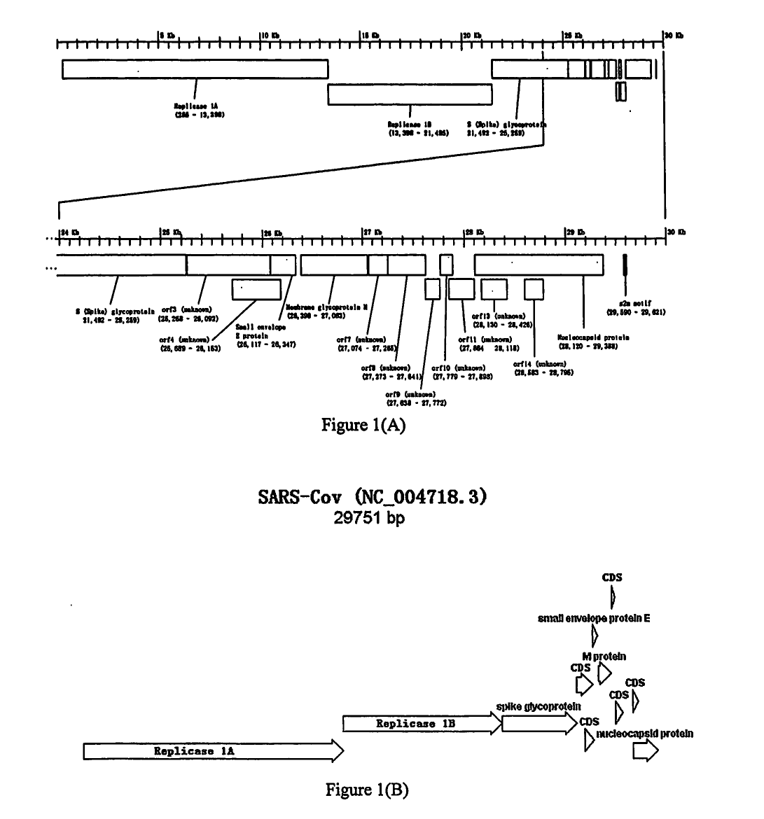

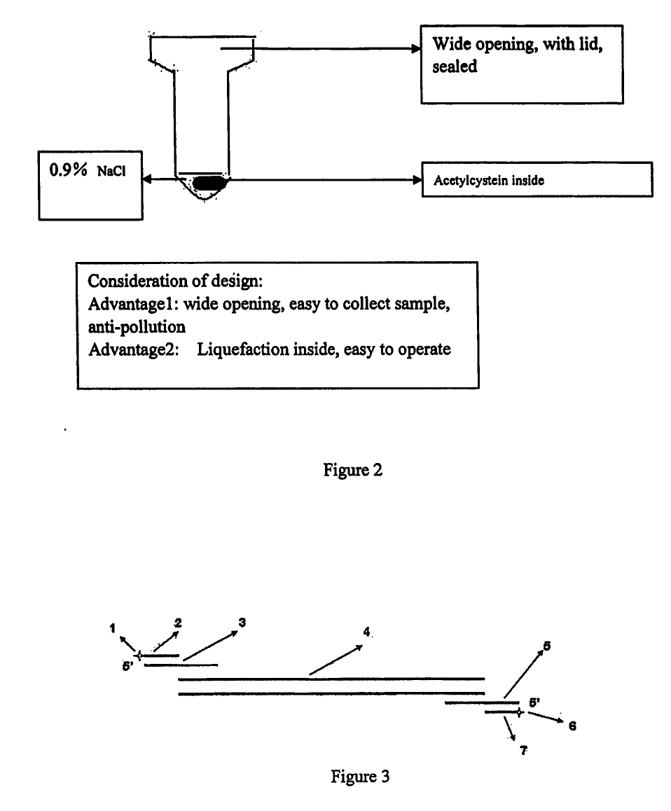

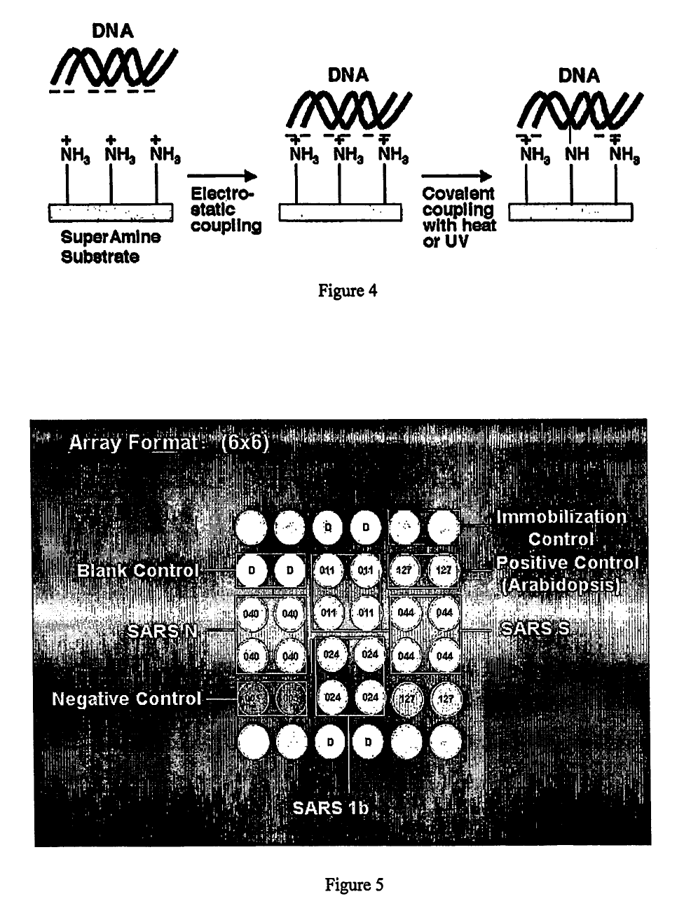

Since SARS coronaviruse is conservative as shown in Tables 22 and 23, nucleic acid based detection methods are rational. FIG. 1B indicates that detection of different parts of SARS coronaviruse genome at the same time can significantly increase the sensitivity and specificity of the detection method.

We have two overall designs. One design is to perform a multiplex PCR for different parts of SARS coronaviruse genome and use PCR products as probes for detection. The second design is to perform a multiplex PCR for different parts of SARS coronaviruse genome and use a 70 mer oligonucleotides as probes for detection.

Target Gene Selection

Based on analysis of SARS coronaviruse genome, we selected three genes as target genes. These three genes are orf 1A and 1B polymerase proteins, spike protein, and nucleocapsid protein. We selected human housekeeping gene GAPD (glyceraldehyde 3-phosphate dehydrogenase) (GenBank Acc: NM_{—}002046) as a positive control for RNA isolation. We selected a gene (Arabidopsis) (GenBank Acc: AJ441252), which has no homology to nucleotide sequence of human and common pathogens, as incorporated positive control.

Design of Primers and Probes

The three proteins of SARS coronaviruse were analyzed and their conservative sequences were compared. According to the requirement of multiplex PCR, multiple pairs of primers, which have similar Tm values and are 1.5 Kb in distance, and have amplified products between 200 to 900 bp, were designed based on the conservative sequence between different genomes. In addition, multiple non-overlapping oligonucleotides (70 mer) were designed based on amplified product of each pair of primers. These primers and probes were compared with the most updated NCBI nucleic acid non-redundant nucleotide database using BLASTN, and the specificities of the probes and primers were assured.

### Example 2

### Process for Pretreatment of Blood Sample

Pretreatment of blood sample involves relatively complicated processes. However, considering the relative low concentration SARS virus in serum reported, pretreatment described herein can effectively enrich lymphocytes from about 2 ml of the whole blood in order to increase the chances of detection.

1. Sample Collection and Transfer

1) Samples collected from patients in the hospital room are put in a first transfer window. The door of the window is then closed and locked.

2) The samples are then transferred into a second transfer window. The samples are recorded in a notebook and three bar code labels are printed. The samples are tested for conventional detection and transferred into a pretreatment transfer window.

2. Use of Biosafe Cabinet

1) Hospital personnel for performing pretreatment process enters the pretreatment room and close the door. The biosafe cabinet is then turned on. The fan of the cabinet and light are then automatically turned on.

2) The indicator lights for power switch, air speed switch, and work light switch are checked for normal operation. The indicator light for air selection switch is checked as off status. Abnormal or unusual operation is reported.

3) The indicator light for alarm switch will make an alarm sound which indicates normal status of the biosafe cabinet after self-testing. Fifteen minutes later, the alarm sound from the indicator light for alarm switch is stopped and the process in the biosafe cabinet can be started.

4) The process in the cabinet cannot be started if the alarm sound continues or the process has to be stopped if there is an alarm sound during the process. The incident has to be reported immediately.

5) After the biosafe cabinet operates normally, samples are taken from the second transfer window and placed in the cabinet. The transfer window top is cleaned by wiping with 75% alcohol and spraying with 0.5% peracetic acid. The door for the transfer window is then closed and locked.

6) The complete process of sample pretreatment is then performed in the biosafe cabinet.

3. Serum Isolation

1) Blood (1.8 ml) with anticoagulant is centrifuged for 10 minutes at 3,500 rpm. The top layer is marked with a marker pen.

2) The top layer serum (about 1.0 ml) is then collected and put into a 1.5 ml sterile Eppendorf centrifuge tube.

3) The Eppendorf centrifuge tube is labeled with the bar code (marked as “P”) and labeled with a sequence number.

4) The sample is then recorded in a notebook.

5) The centrifuge tube containing the serum sample is put in a specialized sample box and stored at −80° C. The outside of the sample box is labeled with SARS, serum and range of sample numbers.

4. Isolation of Blood Cells

1) Lymphocyte isolation solution (3.6 ml) is added to a 10 ml centrifuge tube.

2) Sterile physiological saline (a volume equal to the serum taken out in the centrifuge tube described above) is added to the centrifuge tube containing the blood cells. The blood cells are resuspended in saline using Pasteur pipette.

3) The resuspended blood cells are slowly loaded on top of the lymphocyte isolation solution and centrifuged for 20 minutes at 1,500 rpm.

4) The cells located between the layers are collected and put in a 1.5 ml sterile Eppendorf centrifuge tube, which is then centrifuged for 5 minutes at 10,000 rpm to spin down the cells. The supernatant is withdrawn.

5) The tube containing the cell pellet is then labeled with the bar code (marked “C”) and labeled with a sequence number.

6) The sample is recorded in a notebook.

7) The centrifuge tube containing the blood cell sample is put in a specialized sample box and stored at −80° C. The outside of the sample box is labeled with SARS, blood cells, and range of sample numbers.

8) The glass face plate of the biosafe cabinet is then opened. The bench surface and other surfaces in the biosafe cabinet are then sterilized by wiping with 70% alcohol and spraying 0.5% peracetic acid.

9) After cleaning, the glass face plate is closed. The ultraviolet light is placed inside the cabinet and turned on for 15 minutes.

10) The power switch of the biosafe cabinet is turned off before leaving the sample pretreatment room.

5. Matters Needing Attention

1) The lymphocyte isolation solution should not be used immediately after being taken out of the refrigerator. The solution should be used after its temperature reaches room temperature and the solution is mixed well.

2) The whole isolation process should be performed at 18-28° C. Too high or too low temperature can impact on the quality of isolation process.

3) The pipette tips, Eppendorf centrifuge tubes, gloves, and disposed reagents or liquids should be discarded in a waist tank (containing 0.5% peracetic acid). Everything in the waster tank should be treated at high pressure after experiment and then discarded.

4) 0.5% of peracetic acid is prepared by diluting 32 ml of 16% of peracetic acid in H₂O to make a final volume of 1,000 ml.

### Example 3

### Process for Extracting RNA Using QIAamp Viral RNA Kit

The following procedures are used in RNA preparation:

1. Pipet 560 μl of prepared Buffer AVL containing Carrier RNA into a 1.5-ml microcentrifuge tube. If the sample volume is larger than 140 μl, increase the amount of Buffer AVL/Carrier RNA proportionally (e.g., a 280-μl sample will require 1120 μl Buffer AVL/Carrier RNA).

2. Add 140 μl plasma, serum, urine, cell-culture supernatant, or cell-free body fluid to the Buffer AVL/Carrier RNA in the microcentrifuge tube. Mix by pulse-vortexing for 15 sec. To ensure efficient lysis, it is essential that the sample is mixed thoroughly with Buffer AVL to yield a homogeneous solution. Frozen samples that have only been thawed once can also be used.

3. Incubate at room temperature (15-25° C.) for 10 min. Viral particle lysis is complete after lysis for 10 min at room temperature. Longer incubation times have no effect on the yield or quality of the purified RNA. Potentially infectious agents and RNases are inactivated in Buffer AVL.

4. Briefly centrifuge the 1.5-ml microcentrifuge tube to remove drops from the inside of the lid.

5. Add 560 μl of ethanol (96-100%) to the sample, and mix by pulse-vortexing for 15 sec. After mixing, briefly centrifuge the 1.5-ml microcentrifuge tube to remove drops from inside the lid. Only ethanol is preferred since other alcohols may result in reduced RNA yield and purity. If the sample volume is greater than 140 μl, increase the amount of ethanol proportionally (e.g., a 280-μl sample will require 1120 μl of ethanol). In order to ensure efficient binding, it is essential that the sample is mixed thoroughly with the ethanol to yield a homogeneous solution.

6. Carefully apply 630 μl of the solution from step 5 to the QIAamp spin column (in a 2-ml collection tube) without wetting the rim. Close the cap, and centrifuge at 6000×g (8000 rpm) for 1 min. Place the QIAamp spin column into a clean 2-ml collection tube, and discard the tube containing the filtrate. Close each spin column in order to avoid cross-contamination during centrifugation. Centrifugation is performed at 6,000×g(8,000 rpm) in order to limit microcentrifuge noise. Centrifugation at full speed will not affect the yield or purity of the viral RNA. If the solution has not completely passed through the membrane, centrifuge again at a higher speed until all of the solution has passed through.

7. Carefully open the QIAamp spin column, and repeat step 6. If the sample volume is greater than 140 μl, repeat this step until all of the lysate has been loaded onto the spin column.

8. Carefully open the QIAamp spin column, and add 500 μl of Buffer AW1. Close the cap, and centrifuge at 6,000×g (8,000 rpm) for 1 min. Place the QIAamp spin column in a clean 2-mi collection tube (provided), and discard the tube containing the filtrate. It is not necessary to increase the volume of Buffer AW1 even if the original sample volume was larger than 140 μl.

9. Carefully open the QIAamp spin column, and add 500 μl of Buffer AW2. Close the cap and centrifuge at full speed (20,000×g; 14,000 rpm) for 3 min. Continue directly with step 10, or to eliminate any chance of possible Buffer AW2 carryover, perform step 9a, and then continue with step 10. Note: Residual Buffer AW2 in the eluate may cause problems in downstream applications. Some centrifuge rotors may vibrate upon deceleration, resulting in flow-through, containing Buffer AW2, contacting the QIAamp spin column. Removing the QIAamp spin column and collection tube from the rotor may also cause flowthrough to come into contact with the QIAamp spin column. In these cases, the optional step 9a should be performed. 9a. (Optional): Place the QIAamp spin column in a new 2-ml collection tube (not provided), and discard the old collection tube with the filtrate. Centrifuge at full speed for 1 min.

10. Place the QIAamp spin column in a clean 1.5-ml microcentrifuge tube (not provided). Discard the old collection tube containing the filtrate. Carefully open the QIAamp spin column and add 60 μl of Buffer AVE equilibrated to room temperature. Close the cap, and incubate at room temperature for 1min. Centrifuge at 6,000×g(8,000 rpm) for 1 min. A single elution with 60 μl of Buffer AVE is sufficient to elute at least 90% of the viral RNA from the QIAamp spin column. Performing a double elution using 2×40 μl of Buffer AVE will increase yield by up to 10%. Elution with volumes of less than 30 μl will lead to reduced yields and will not increase the final concentration of RNA in the eluate. Viral RNA is stable for up to one year when stored at −20° C. or −70° C.

The following are further information pertaining to the above procedures:
Equilibrate samples to room temperature (15-25° C.).
Equilibrate Buffer AVE to room temperature for elution in step 10.
Check whether Buffer AW1, Buffer AW2, and Carrier RNA have been prepared according to the instructions on pages 14-15.
Redissolve precipitate in Buffer AVL/Carrier RNA by heating, if necessary, and cool to room temperature before use.
All centrifugation steps are carried out at room temperature.

### Example 4

### An Exemplary Array Format of SARS-CoV Detection Chip

FIG. 5 illustrates an exemplary array format of SARS-CoV detection chip.

Immobilization control is an oligo-probe that is labeled by a fluorescent dye HEX on its end and does not participate in any hybridization reaction when a sample containing or suspected of containing of a SARS-CoV is contacted with the chip.

Positive control(Arabidopsis) is an oligo-probe designed according to an Arabidopsis (one kind of model organism) gene and does not participate in any hybridization reaction when a sample containing or suspected of containing of a SARS-CoV is contacted with the chip. During hybridization reaction, target probes that can hybridize with this positive control perfectly are added into the hybridization solution. Signals of the positive control can be applied to monitor the hybridization reaction.

Negative control is an oligo-probe that does not participate in any hybridization reaction when a sample containing or suspected of containing of a SARS-CoV is contacted with the chip.

Blank Control is DMSO solution spot. It is used for monitoring arraying quality.

SARS probes are 011, 024, 040 and 044 probes.

### Example 5

### SARS-CoV Detection From a SARS Patient Blood Sample (Sample No. 3)

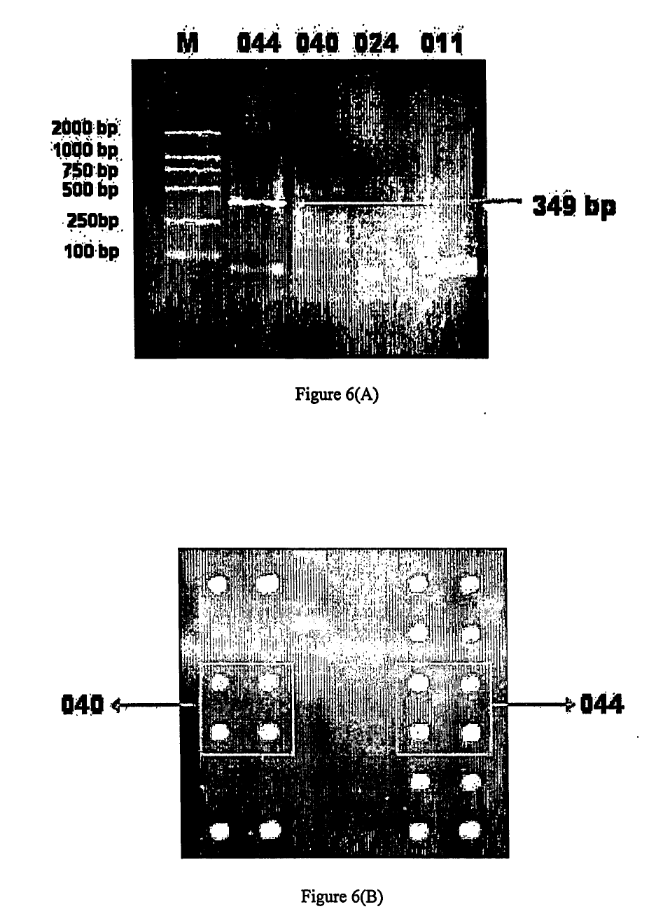

FIGS. 6A and 6B illustrate SARS-CoV detection from a SARS patient blood sample (sample No. 3). Lymphocytes were isolated from 3# SARS patient blood sample. RNA from lymphocytes was extracted by QIAamp Kit. RT-nest PCR was performed using RNA extracted above as templates. 044 RT-nest PCR result was good and hybridization result was good too. 040 RT-nest PCR result was poor but hybridization result was good. It shows that the chip-hybridization method is sensitive and specific.

### Example 6

### SARS-CoV Detection from a SARS Patient Blood Sample (Sample No. 4)

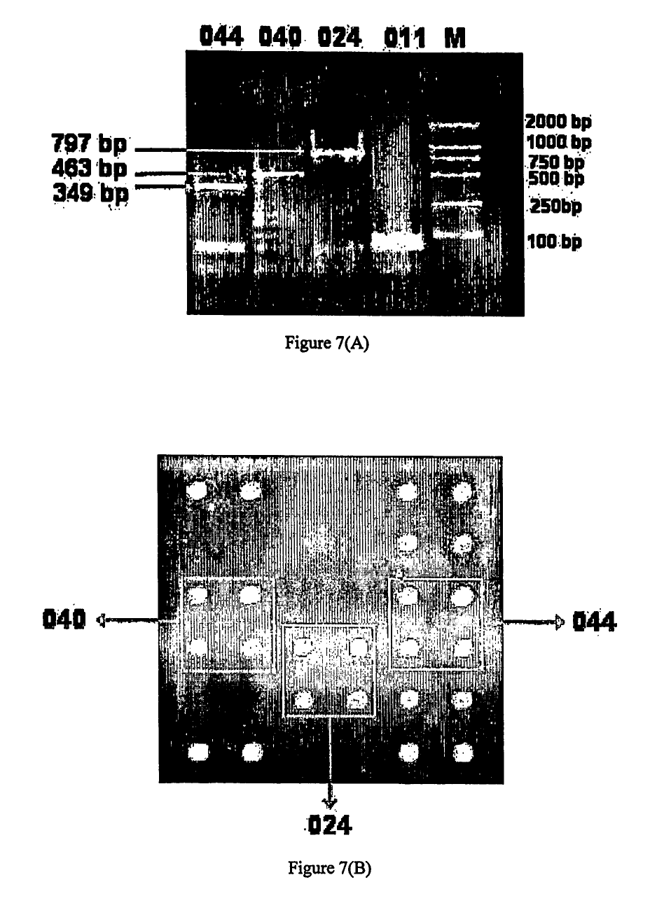

FIGS. 7A and 7B illustrate SARS-CoV detection from a SARS patient blood sample (sample No. 4). Lymphocytes were isolated from 4# SARS patient blood sample. RNA from lymphocytes was extracted by QIAamp Kit. RT-nest PCR was performed using RNA extracted above as templates. 024, 040 and 044 RT-nest PCR results were good and hybridization results were good too.

### Example 7

### SARS-CoV Detection from a SARS Patient Sputum Sample (Sample No. 5)

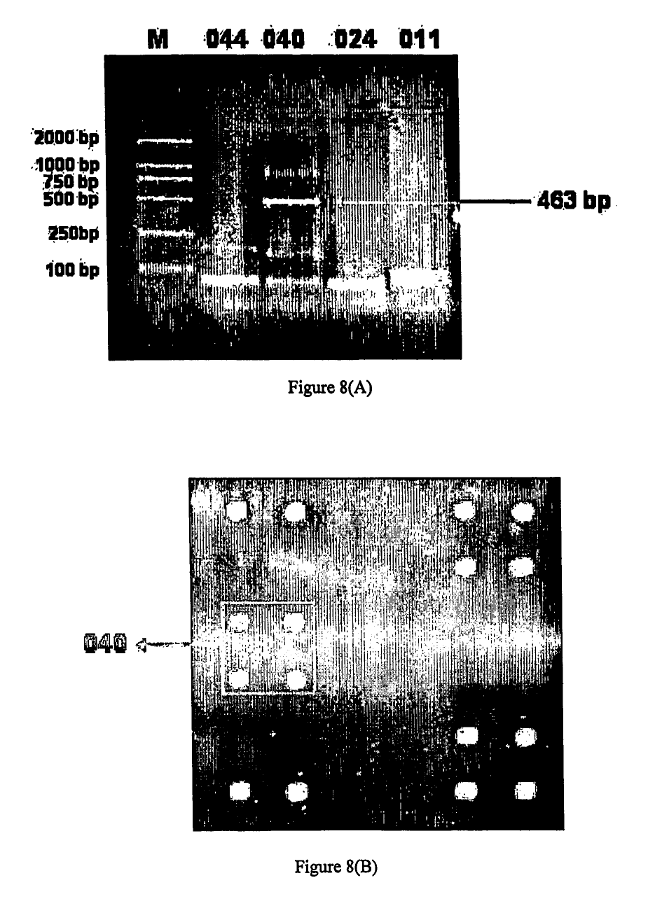

FIG. 8 illustrates SARS-CoV detection from a SARS patient sputum sample (sample No. 5). RNA from 5# SARS patient sputum sample was extracted by QIAamp Kit. RT-nest PCR was performed using RNA extracted above as templates. 040 RT-nest PCR result was good and hybridization result was good too.

### Example 8

### SARS-CoV Detection from a SARS Patient Sputum Sample (Sample No. 6)

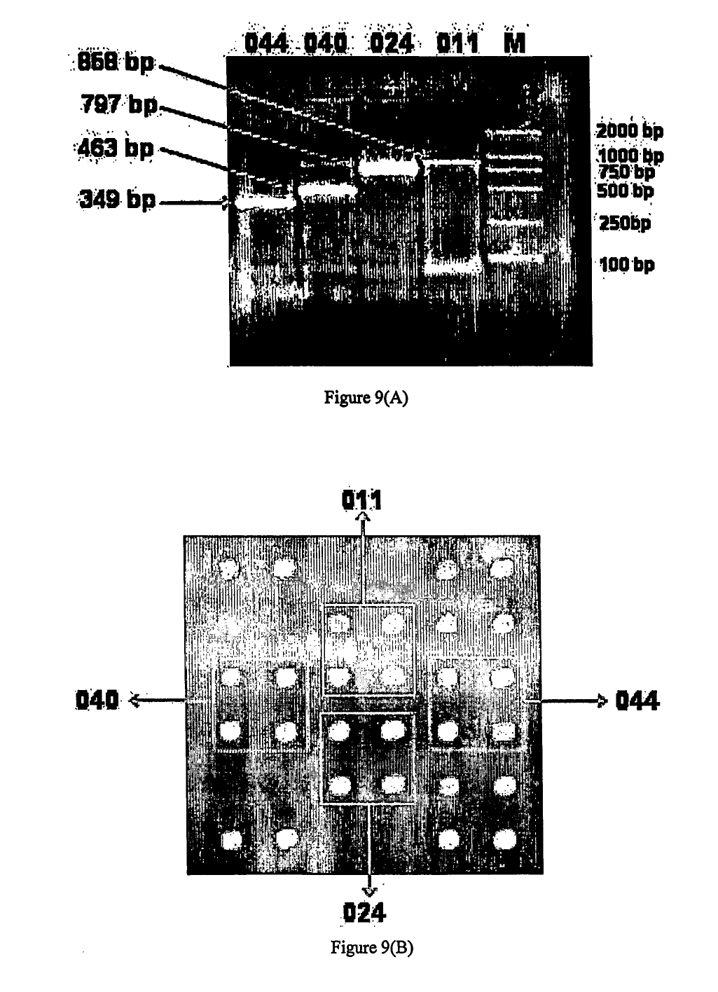

FIG. 9 illustrates SARS-CoV detection from a SARS patient sputum sample (sample No. 6). RNA from 6# SARS patient sputum sample was extracted by QIAamp Kit. RT-nest PCR was performed using RNA extracted above as templates. All probes RT-nest PCR results were good and hybridization results were good too.

### Example 9

### Another Exemplary Array Format of SARS-CoV Detection Chip

FIG. 10 illustrates another exemplary array format of SARS-CoV detection chip.

Immobilization control is an oligo-probe that is labeled by a fluorescent dye HEX on its end and does not participate in any hybridization reaction when a sample containing or suspected of containing of a SARS-CoV is contacted with the chip.

Positive control (Arabidopsis) is an oligo-probe designed according to an Arabidopsis (one kind of model organism) gene and does not participate in any hybridization reaction when a sample containing or suspected of containing of a SARS-CoV is contacted with the chip. During hybridization reaction, target probes that can hybridize with this positive control perfectly are added into the hybridization solution. Signals of the positive control can be applied to monitor the hybridization reaction.

Negative control is an oligo-probe that does not participate in any hybridization reaction when a sample containing or suspected of containing of a SARS-CoV is contacted with the chip.

Blank Control is DMSO solution spot. It is used for monitoring arraying quality.

SARS probes are 011, 024, 040 and 044 probes.

### Example 10.

### Possible Positive Results on the SARS-CoV Detection Chip Illustrated in FIG. 10

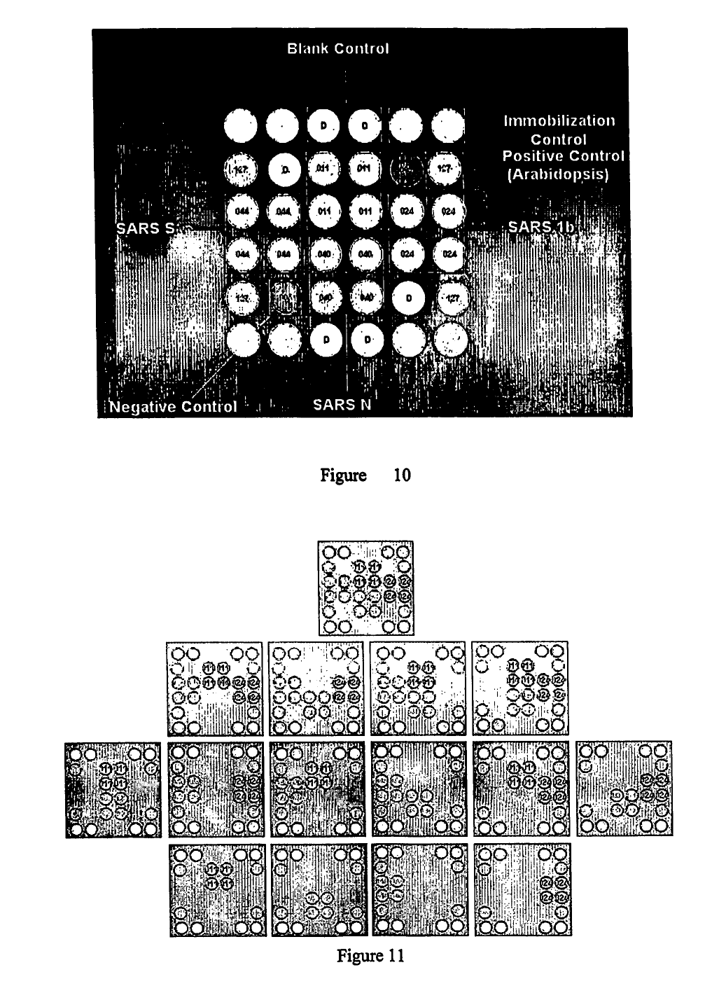

FIG. 11 illustrates all possible positive results on the SARS SARS-CoV detection chip illustrated in FIG. 10.

There are four sets probes on chips for detecting SARS virus: probe 011, probe 024, probe 040 and probe 044.

The first line gives the positive result (1) by signals appearing on all four sets of probes: 011+024+040+044.

The second line gives all the possible positive results (4) by signals appearing on three sets probes: 011+024+044, 024+040+044, 011+040+044, 011+024+040.

The third line gives all the possible positive results (6) by signals appearing on two sets probes: 011+040, 024+044, 011+044, 040+044, 011+024, 024+040.

The fourth line gives all the possible positive results (4) by signals appearing on only one set probes: 011, 024, 040, 044.

### Example 11

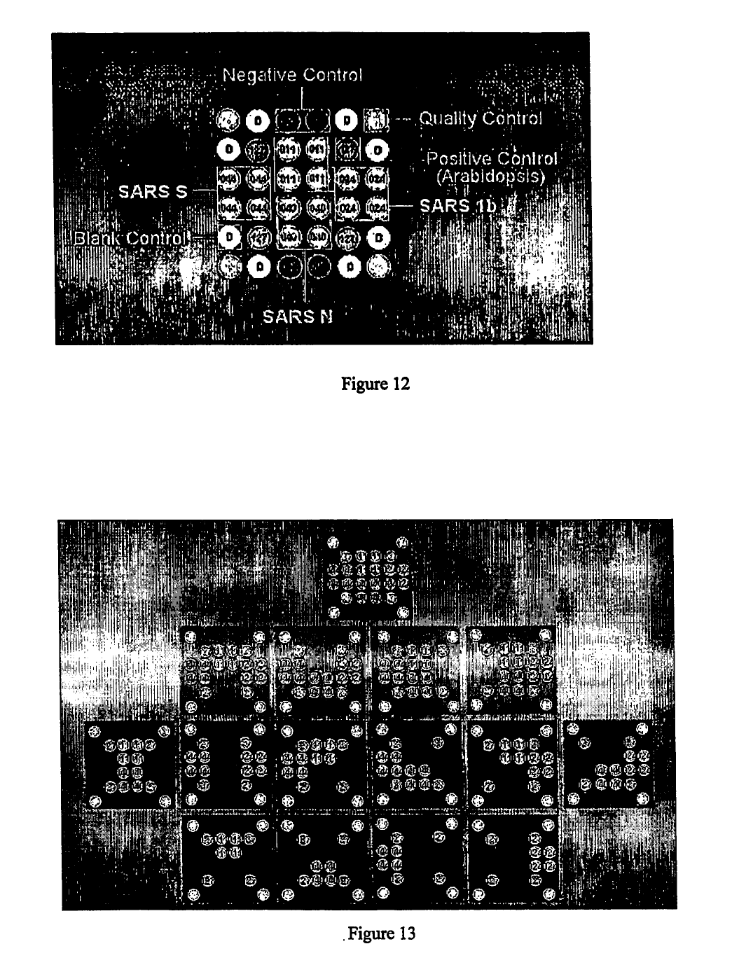

### Possible Results on the SARS-CoV Detection Chip Illustrated in FIG. 12

FIG. 13 illustrates all possible positive results on the SARS-CoV detection chip illustrated in FIG. 12.

There are four sets of probes on chips for detecting SARS virus: probe 011, probe 024, probe 040 and probe 044.

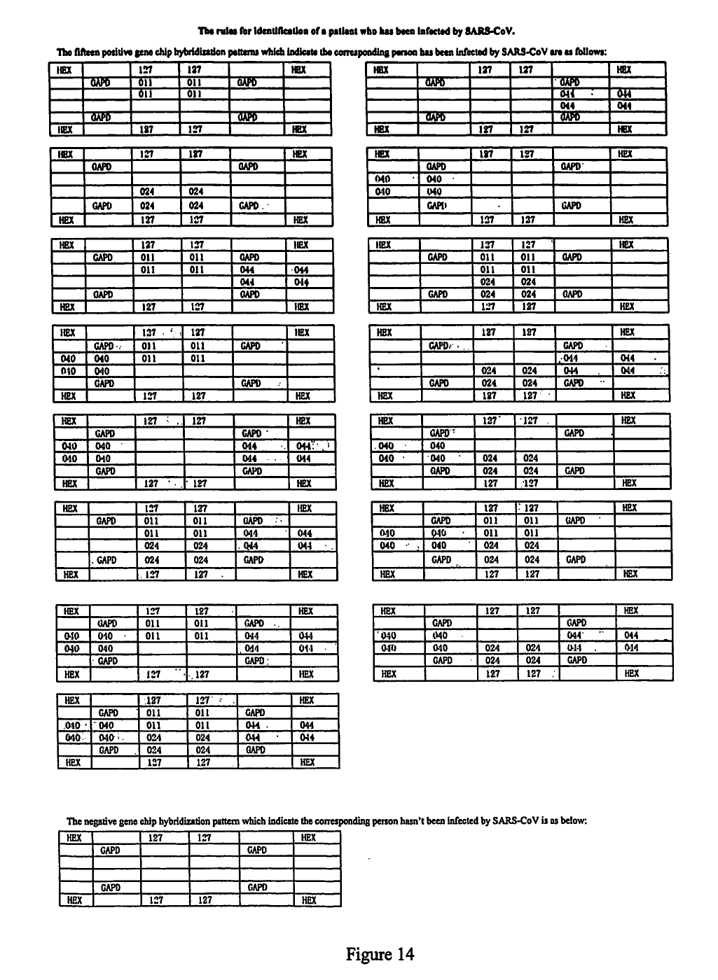

The possible positive and negative results are also illustrated in FIG. 14. The combinations for positive results include:

011+127;
040 +127;
011+127+024;
011+127+044;
024+127+044;
011+127+024+040;
024+127;
044+127;
011+127+040;
024+127+040;
044+127+040;
011+127+044;
011+127+024+044;
011+127+024+040+044; and
127+024+040+044.

A negative result is indicated if only 127 is observed.

To be a valid assay result, positive or negative, the immobilization control signal (HEX should always be observed.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A chip for assaying for a coronaviruse causing the severe acute respiratory syndrome (SARS-CoV) and a non-SARS-CoV infectious organism, which chip comprises a support suitable for use in nucleic acid hybridization having immobilized thereon an oligonucleotide probe complementary to a nucleotide sequence of SARS-CoV genome, said nucleotide sequence comprising at least 10 nucleotides, and one or more of the following oligonucleotide probe(s):
a) an oligonucleotide probe complementary to a nucleotide sequence of a non-SARS-CoV infectious organism causing SARS-like symptoms, said nucleotide sequence comprising at least 10 nucleotides;
b) an oligonucleotide probe complementary to a nucleotide sequence of a non-SARS-CoV infectious organism damaging an infectious host's immune system, said nucleotide sequence comprising at least 10 nucleotides; or
c) an oligonucleotide probe complementary to a nucleotide sequence of a non-SARS-CoV coronaviridae virus, said nucleotide sequence comprising at least 10 nucleotides.

2. The chip of claim 1, which chip comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

3. The chip of claim 2, wherein the at least two different nucleotide sequences of SARS-CoV genome comprises:
a) a nucleotide sequence of at least 10 nucleotides located within a conserved region of SARS-CoV genome and a nucleotide sequence of at least 10 nucleotides located within a variable region of SARS-CoV genome; or
b) a nucleotide sequence of at least 10 nucleotides located within a structural protein coding gene of SARS-CoV genome and a nucleotide sequence of at least 10 nucleotides located within a non-structural protein coding gene of SARS-CoV genome.

4. The chip of claim 2, which further comprises:
a) at least one of the following three oligonucleotide probes: an immobilization control probe that is labeled and does not participate in any hybridization reaction when a sample containing or suspected of containing of a SARS-CoV or a non-SARS-CoV infectious organism is contacted with the chip, a positive control probe that is not complementary to any sequence of a SARS-CoV or non-SARS-CoV infectious organism but is complementary to a sequence contained in the sample not found in the SARS-CoV or the non-SARS-CoV infectious organism and a negative control probe that is not complementary to any nucleotide sequence contained in the sample; and
b) a blank spot.

5. The chip of claim 2, which comprises at least two oligonucleotide probes complementary to two different nucleotide sequences of at least 10 nucleotides, respectively, located within a conserved region of SARS-CoV genome, located within a structural protein coding gene of SARS-CoV genome or located within a non-structural protein coding gene of SARS-CoV genome.

6. The chip of claim 2, wherein:
a) the conserved region of SARS-CoV genome is a region located within the Replicase 1A or 1B gene or the Nucleocapsid (N) gene of SARS-CoV;
b) the structural protein coding gene of SARS-CoV genome is a gene encoding the Spike glycoprotein (S), the small envelope protein (E) or the Nucleocapsid protein (N); or
c) the non-structural protein coding gene of SARS-CoV genome is a gene encoding the Replicase 1A or 1B.

7. The chip of claim 2, wherein the variable region of SARS-CoV genome is a region located within the Spike glycoprotein (S) gene of SARS-CoV.

8. The chip of claim 2, which comprises at least two of the following four oligonucleotide probes: two oligonucleotide probes complementary to two different nucleotide sequences of at least 10 nucleotides located within the Replicase 1A or 1B gene of SARS-CoV, an oligonucleotide probe complementary to a nucleotide sequence of at least 10 nucleotides located within the N gene of SARS-CoV and an oligonucleotide probe complementary to a nucleotide sequence of at least 10 nucleotides located within the S gene of SARS-CoV.

9. The chip of claim 8, wherein one of the two different nucleotide sequences located within the Replicase 1A or 1B gene of SARS-CoV comprises a nucleotide sequence that:
a) hybridizes, under high stringency, with a Replicase 1A or 1B nucleotide sequence, or a complementary strand thereof, that is set forth in Table 13; or
b) has at least 90% identity to a Replicase 1A or 1B nucleotide sequence comprising a nucleotide sequence, or a complementary strand thereof, that is set forth in Table 13.

10. The chip of claim 9, wherein one of the two different nucleotide sequences located within the Replicase 1A or 1B gene of SARS-CoV comprises a nucleotide sequence that is set forth in Table 13.

11. The chip of claim 8, wherein the nucleotide sequence located within the N gene of SARS-CoV comprises a nucleotide sequence that:
a) hybridizes, under high stringency, with a N nucleotide sequence, or a complementary strand thereof, that is set forth in Table 13; or
b) has at least 90% identity to a N nucleotide sequence comprising a nucleotide sequence, or a complementary strand thereof, that is set forth in Table 13.

12. The chip of claim 11, wherein the nucleotide sequence located within the N gene of SARS-CoV comprises a nucleotide sequence that is set forth in Table 13.

13. The chip of claim 8, wherein the nucleotide sequence located within the S gene of SARS-CoV comprises a nucleotide sequence that:
a) hybridizes, under high stringency, with a S nucleotide sequence, or a complementary strand thereof, that is set forth in Table 13; or
b) has at least 90% identity to a S nucleotide sequence comprising a nucleotide sequence, or a complementary strand thereof, that is set forth in Table 13.

14. The chip of claim 13, wherein the nucleotide sequence located within the S gene of SARS-CoV comprises a nucleotide sequence that is set forth in Table 13.

15. The chip of claim 4, wherein the label of the immobilization control probe is selected from the group consisting of a chemical, an enzymatic, an immunogenic, a radioactive, a fluorescent, a luminescent and a FRET label.

16. The chip of claim 4, wherein the non-SARS-CoV-sequence is spiked in the sample to be assayed.

17. The chip of claim 16, wherein the spiked non-SARS-CoV-sequence is a sequence of Arabidopsis origin.

18. The chip of claim 8, which comprises two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe that is labeled and does not participate in any hybridization reaction when a sample containing or suspected of containing of a SARS-CoV or a non-SARS-CoV infectious organism is contacted with the chip, a positive control probe that is not complementary to any sequence of a SARS-CoV or non-SARS-CoV infectious organism but is complementary to a sequence contained in the sample not found in the SARS-CoV or the non-SARS-CoV infectious organism and a negative control probe that is not complementary to any nucleotide sequence contained in the sample.

19. The chip of claim 18, which comprises multiple spots of the two oligonucleotide probes complementary to two different nucleotide sequences located within the Replicase 1B gene of SARS-CoV, the oligonucleotide probe complementary to a nucleotide sequence located within the N gene of SARS-CoV, the oligonucleotide probe complementary to a nucleotide sequence located within the S gene of SARS-CoV, the immobilization control probe, the positive control probe and the negative control probe.

20. The chip of claim 4, wherein at least one of the oligonucleotide probe comprises, at its 5′ end, a poly dT region to enhance its immobilization on the support.

21. The chip of claim 2, wherein at least one of the oligonucleotide probes is complementary to a highly expressed nucleotide sequence of SARS-CoV genome.

22. The chip of claim 1, wherein the non-SARS-CoV infectious organism causing SARS-like symptoms is selected from the group consisting of a human coronaviruse 229E, a human coronaviruse OC43, a human enteric coronaviruse, an influenza virus, a parainfluenza virus, a respiratory sncytical virus, a human metapneumovirus, a rhinovirus, an adenoviruse, a mycoplasma pneumoniae, a chlamydia pneumoniae, a measles virus and a rubella virus.

23. The chip of claim 22, wherein the influenza virus is influenza virus A or influenza virus B.

24. The chip of claim 22, wherein the parainfluenza virus is selected from the group consisting of parainfluenza virus 1, parainfluenza virus 2, parainfluenza virus 3 and parainfluenza virus 4.

25. The chip of claim 1, wherein the non-SARS-CoV infectious organism damaging an infectious host's immune system is selected from the group consisting of a hepatitis virus, a transfusion transmitting virus (TTV), a human immunodeficiency virus (HIV), a parvovirus, a human cytomegalovirus (HCMV), an Epstein-Barr virus (EBV) and a tre-ponema palidum.

26. The chip of claim 25, wherein the hepatitis virus is selected from the group consisting of hepatitis virus A (HAV), hepatitis virus B (HBV), hepatitis virus C (HCV), hepatitis virus D (HDV), hepatitis virus E (HEV) and hepatitis virus G (HGV).

27. The chip of claim 25, wherein the HIV is HIV I.

28. The chip of claim 25, wherein the parvovirus is parvovirus B19.

29. The chip of claim 1, wherein the non-SARS-CoV coronaviridae virus is selected from the group consisting of an avian infectious bronchitis virus, an avian infectious laryngotracheitis virus, a murine hepatitis virus, an equine coronaviruse, a canine coronaviruse, a feline coronaviruse, a porcine epidemic diarrhea virus, a porcine transmissible gastroenteritis virus, a bovine coronaviruse, a feline infectious peritonitis virus, a rat coronaviruse, a neonatal calf diarrhea coronaviruse, a porcine hemagglutinating encephalomyelitis virus, a puffinosis virus, a turkey coronaviruse and a sialodacryoadenitis virus of rat.

30. The chip of claim 1, wherein the support comprises a surface that is selected from the group consisting of a silicon, a plastic, a glass, a ceramic, a rubber, and a polymer surface.

31. A method for assaying for a SARS-CoV and a non-SARS-CoV infectious organism in a sample, which methods comprises:
a) providing a chip of claim 1;
b) contacting said chip with a sample containing or suspected of containing a nucleotide sequence of a SARS-CoV and a non-SARS-CoV infectious organism under conditions suitable for nucleic acid hybridization; and
c) assessing hybrids formed between said nucleotide sequence of said SARS-CoV or said non-SARS-CoV infectious organism, if present in said sample, and said oligonucleotide probe complementary to a nucleotide sequence of said SARS-CoV genome or said oligonucleotide probe complementary to a nucleotide sequence of said non-SARS-CoV infectious organism genome,
whereby detection of one or both of said hybrids indicates the presence of said SARS-CoV and/or said non-SARS-CoV infectious organism in said sample.

32. The method of claim 31, wherein the SARS-CoV is assayed by:
a) providing a chip of claim 2;
b) contacting said chip with a sample containing or suspected of containing a SARS-CoV nucleotide sequence under conditions suitable for nucleic acid hybridization; and
c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample,
whereby detection of one or both said hybrids indicates the presence of said SARS-CoV in said sample.

33. The method of claim 31, wherein the SARS-CoV is assayed by:
a) providing a chip of claim 3;
b) contacting said chip with a sample containing or suspected of containing a SARS-CoV nucleotide sequence under conditions suitable for nucleic acid hybridization; and
c) assessing hybrids formed between said SARS-CoV nucleotide sequence, if present in said sample, and
i) said oligonucleotide probe complementary to a nucleotide sequence located within a conserved region of SARS-CoV genome and an oligonucleotide probe complementary to a nucleotide sequence located within a variable region of SARS-CoV genome, respectively; or
ii) said oligonucleotide probe complementary to a nucleotide sequence located within a structural protein coding gene of SARS-CoV genome and an oligonucleotide probe complementary to a nucleotide sequence located within a non-structural protein coding gene of SARS-CoV genome,
to determine the presence, absence or amount of said SARS-CoV in said sample,
whereby detection of one or both said hybrids indicates the presence of said SARS-CoV in said sample.

34. The method of claim 31, wherein the SARS-CoV is assayed by:
a) providing a chip of claim 4;
b) contacting said chip with a sample containing or suspected of containing a SARS-CoV nucleotide sequence under conditions suitable for nucleic acid hybridization; and
c) assessing:
(i) hybrids formed between said SARS-CoV nucleotide sequence, if present in the sample, and the oligonucleotide probe complementary to a nucleotide sequence within a conserved region of SARS-CoV genome and an oligonucleotide probe complementary to a nucleotide sequence located within a variable region of SARS-CoV genome, respectively;
(ii) a label comprised in the immobilization control probe, or a hybrid(s) involving the positive control probe and/or the negative control probe; and
(iii) a signal at said blank spot
to determine the presence, absence or amount of said SARS-CoV in a sample.

35. The method of claim 34, wherein the chip comprises two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe, a positive control probe and a negative control probe and the presence of the SARS-CoV is determined when:
a) a positive hybridization signal is detected using at least one of the two oligonucleotide probes complementary to two different nucleotide sequences located within the Replicase 1A or 1B gene of SARS-CoV, the oligonucleotide probe complementary to a nucleotide sequence located within the N gene of SARS-CoV and/or the oligonucleotide probe complementary to a nucleotide sequence located within the S gene of SARS-CoV;
b) a positive signal is detected from the immobilization control probe;
c) a positive hybridization signal is detected using the positive control probe;
d) a positive hybridization signal is not detected using the negative control probe; and
e) a positive hybridization signal is not detected at the blank spot.

36. The method of claim 35, wherein detecting a positive hybridization signal using at least one of the two oligonucleotide probes complementary to two different nucleotide sequences located within the Replicase 1A or 1B gene of SARS-CoV, or the oligonucleotide probe complementary to a nucleotide sequence located within the N gene of SARS-CoV, while not detecting a positive hybridization signal using the oligonucleotide probe complementary to a nucleotide sequence located within the S gene of SARS-CoV indicates mutation of the SARS-CoV.

37. The method of claim 31, wherein the chip of claim 21 is used and the method is used to diagnose early-stage SARS patients.

38. The method of claim 37, wherein the early-stage SARS patients have been infected with SARS-CoV from about less than one day to about three days.

39. The method of claim 31, which is used to determine whether a subject is infected by a SARS-CoV and/or a non-SARS-CoV infectious organism causing SARS-like symptoms.

40. The method of claim 39, wherein the SARS-like symptoms are caused by a non-SARS-CoV infectious organism selected from the group consisting of a human coronaviruse 229E, a human coronaviruse OC43, a human enteric coronaviruse, an influenza virus, a parainfluenza virus, a respiratory sncytical virus, a human metapneumovirus, a rhinovirus, an adenoviruse, a mycoplasma pneumoniae, a chlamydia pneumoniae, a measles virus and a rubella virus.

41. The method of claim 40, wherein the influenza virus is influenza virus A or influenza virus B.

42. The method of claim 40, wherein the parainfluenza virus is selected from the group consisting of parainfluenza virus 1, parainfluenza virus 2, parainfluenza virus 3 and parainfluenza virus 4.

43. The method of claim 31, which is used to determine whether a subject is infected by a SARS-CoV and/or a non-SARS-CoV infectious organism damaging the subject's immune system.

44. The method of claim 43, wherein the non-SARS-CoV infectious organism damaging subject's immune system is selected from the group consisting of a hepatitis virus, a transfusion transmitting virus (TTV), a human immunodeficiency virus (HIV), a parvovirus, a human cytomegalovirus (HCMV), an Epstein-Barr virus (EBV) and a tre-ponema palidum.

45. The method of claim 44, wherein the hepatitis virus is selected from the group consisting of hepatitis virus A (HAV), hepatitis virus B (HBV), hepatitis virus C (HCV), hepatitis virus D (HDV), hepatitis virus E (HEV) and hepatitis virus G (HGV).

46. The method of claim 44, wherein the HIV is HIV I.

47. The method of claim 44, wherein the parvovirus is parvovirus B19.

48. The method of claim 31, which is used to determine whether a subject is infected by a SARS-CoV and/or a non-SARS-CoV coronaviridae virus.

49. The method of claim 48, wherein the non-SARS-CoV coronaviridae virus is selected from the group consisting of an avian infectious bronchitis virus, an avian infectious laryngotracheitis virus, a murine hepatitis virus, an equine coronaviruse, a canine coronaviruse, a feline coronaviruse, a porcine epidemic diarrhea virus, a porcine transmissible gastroenteritis virus, a bovine coronaviruse, a feline infectious peritonitis virus, a rat coronaviruse, a neonatal calf diarrhea coronaviruse, a porcine hemagglutinating encephalomyelitis virus, a puffinosis virus, a turkey coronaviruse and a sialodacryoadenitis virus of rat.

50. The method of claim 31, wherein the nucleotide sequence of the SARS-CoV or the non-SARS-CoV infectious organism is a genomic sequence of the SARS-CoV or the non-SARS-CoV infectious organism or a DNA sequence amplified from an extracted SARS-CoV RNA genomic sequence or an extracted genomic sequence of the non-SARS-CoV infectious organism.

51. The method of claim 50, wherein the SARS-CoV RNA genomic sequence is extracted from a SARS-CoV infected cell using the QIAamp Viral RNA kit, the Chomczynski-Sacchi technique or TRIzol.

52. The method of claim 50, wherein the SARS-CoV RNA genomic sequence is extracted from a SARS-CoV infected cell using the QIAamp Viral RNA kit.

53. The method of claim 31, wherein the genomic sequence of the of the SARS-CoV or the non-SARS-CoV infectious organism is extracted from a sputum or saliva sample, a lymphocyte of a blood sample.

54. The method of claim 31, wherein the genomic sequence of the of the SARS-CoV or the non-SARS-CoV infectious organism is extracted from nasopharyngeal, oropharyngeal, tracheal, bronchaleolar lavage, pleural fluid, urine, stool, conjunctiva, tissue from human, mouse, dog, rat, cat, horse, avian, earth, water, air.

55. The method of claim 50, wherein the genomic sequence of the of the SARS-CoV or the non-SARS-CoV infectious organism is amplified by PCR.

56. The method of claim 55, wherein a label is incorporated into the amplified DNA sequence during the PCR.

57. The method of claim 55, wherein the PCR comprises conventional, multiplex, nested PCR or RT-PCR.

58. The method of claim 55, wherein the PCR comprises a two-step nested PCR, the first step being a RT-PCR and the second step being a conventional PCR.

59. The method of claim 55, wherein the PCR comprises a one-step, multiplex RT-PCR using a plurality of 5′ and 3′ specific primers, each of the specific primers comprising a specific sequence complementary to its target sequence to be amplified and a common sequence, and a 5′ and a 3′ universal primer, the 5′ universal primer being complementary to the common sequence of the 5′ specific primers and the 3′ universal primer being complementary to the common sequence of the 3′ specific primers, and wherein in the PCR, the concentration of the 5′ and 3′ universal primers equals to or is higher than the concentration of the 5′ and 3′ specific primers, respectively.

60. The method of claim 59, wherein the 3′ universal primer and/or the 5′ universal primer is labeled.

61. The method of claim 60, wherein the label is a fluorescent label.

62. The method of claim 55, wherein the PCR comprises a multiplex nested PCR.

63. The method of claim 55, wherein the PCR is conducted using at least one of the following pairs of primers set forth in Table 18 or Tables 19-21.

64. An oligonucleotide primer for amplifying a nucleotide sequence of an influenza A virus, an influenza B virus, a human metapneumovirus, a human adenovirus, a human coronaviruse 229E or a human coronaviruse OC43, which oligonucleotide primer comprises a nucleotide sequence that:
a) hybridizes, under high stringency, with a target nucleotide sequence of influenza A virus, influenza B virus, human metapneumovirus, human adenovirus, human coronaviruse 229E or human coronaviruse OC43, or a complementary strand thereof, that is set forth in Tables 1-6; or
b) has at least 90% identity to a target nucleotide sequence of influenza A virus, influenza B virus, human metapneumovirus, human adenovirus, human coronaviruse 229E or human coronaviruse OC43 comprising a nucleotide sequence, or a complementary strand thereof, that is set forth in Tables 1-6.

65. The primer of claim 64, which comprises DNA, RNA, PNA or a derivative thereof.

66. The primer of claim 64, which comprises a nucleotide sequence, or a complementary strand thereof, that is set forth in Tables 1-6.

67. A kit for amplifying a nucleotide sequence of an influenza A virus, an influenza B virus, a human metapneumovirus, a human adenovirus, a human coronaviruse 229E or a human coronaviruse OC43, which kit comprises:
a) a primer of claim 64; and
b) a nucleic acid polymerase that can amplify a nucleotide sequence of an influenza A virus, an influenza B virus, a human metapneumovirus, a human adenovirus, a human coronaviruse 229E or a human coronaviruse OC43 using said primer of claim 64.

68. The kit of claim 67, wherein the nucleic acid polymerase is a reverse transcriptase.

69. An oligonucleotide probe for hybridizing to a nucleotide sequence of an influenza A virus, an influenza B virus, a human metapneumovirus, a human adenovirus, a human coronaviruse 229E or a human coronaviruse OC43, which oligonucleotide probe comprises a nucleotide sequence that:
a) hybridizes, under high stringency, with a target nucleotide sequence of influenza A virus, influenza B virus, human metapneumovirus, human adenovirus, human coronaviruse 229E or human coronaviruse OC43, or a complementary strand thereof, that is set forth in Tables 7-12; or
b) has at least 90% identity to a target nucleotide sequence of influenza A virus, influenza B virus, human metapneumovirus, human adenovirus, human coronaviruse 229E or human coronaviruse OC43, or a complementary strand thereof, that is set forth in Tables 7-12.

70. The probe of claim 69, which comprises DNA, RNA, PNA or a derivative thereof.

71. The probe of claim 69, which comprises a nucleotide sequence, or a complementary strand thereof, that is set forth in Tables 7-12.

72. The probe of claim 69, which is labeled.

73. The probe of claim 72, wherein the label is selected from the group consisting of a chemical, an enzymatic, an immunogenic, a radioactive, a fluorescent, a luminescent and a FRET label.

74. A kit for hybridization analysis of a nucleotide sequence of an influenza A virus, an influenza B virus, a human metapneumovirus, a human adenovirus, a human coronaviruse 229E or a human coronaviruse OC43, which kit comprises:
a) a probe of claim 69; and
b) a means for assessing a hybrid formed between a nucleotide sequence of an influenza A virus, an influenza B virus, a human metapneumovirus, a human adenovirus, a human coronaviruse 229E or a human coronaviruse OC43 and said probe.


SEQUENCE LISTING (945 sequences)

SEQ ID NO: 1 - DNA, 19 bp, Artificial Sequence
  Primer
tttgtgcgacaatgcttca

SEQ ID NO: 2 - DNA, 20 bp, Artificial Sequence
  Primer
gacatttgagaaagcttgcc

SEQ ID NO: 3 - DNA, 20 bp, Artificial Sequence
  Primer
  misc_feature 12: n = a, c, g, or t
agggacaacctngaacctgg

SEQ ID NO: 4 - DNA, 22 bp, Artificial Sequence
  Primer
aggagttgaaccaagacgcatt

SEQ ID NO: 5 - DNA, 22 bp, Artificial Sequence
  Primer
accacattcccttatactggag

SEQ ID NO: 6 - DNA, 24 bp, Artificial Sequence
  Primer
ttagtcatcatctttctcacaaca

SEQ ID NO: 7 - DNA, 22 bp, Artificial Sequence
  Primer
acaaattgcttcaaatgagaac

SEQ ID NO: 8 - DNA, 22 bp, Artificial Sequence
  Primer
tgtctccgaagaaataagatcc

SEQ ID NO: 9 - DNA, 20 bp, Artificial Sequence
  Primer
gcgcagagacttgaagatgt

SEQ ID NO: 10 - DNA, 18 bp, Artificial Sequence
  Primer
ccttccgtagaaggccct

SEQ ID NO: 11 - DNA, 21 bp, Artificial Sequence
  Primer
cacaatggcagaatttagtga

SEQ ID NO: 12 - DNA, 20 bp, Artificial Sequence
  Primer
gtcagtttgatcccgtagtg

SEQ ID NO: 13 - DNA, 20 bp, Artificial Sequence
  Primer
cagatcccagagtggactca

SEQ ID NO: 14 - DNA, 22 bp, Artificial Sequence
  Primer
tgtattacccaagggttgttac

SEQ ID NO: 15 - DNA, 22 bp, Artificial Sequence
  Primer
gatcagcatgacagtaacagga

SEQ ID NO: 16 - DNA, 22 bp, Artificial Sequence
  Primer
atgttcggtaaaagtcgtttat

SEQ ID NO: 17 - DNA, 20 bp, Artificial Sequence
  Primer
ccacaggggagattccaaag

SEQ ID NO: 18 - DNA, 23 bp, Artificial Sequence
  Primer
gacattcttcctgattcataatc

SEQ ID NO: 19 - DNA, 22 bp, Artificial Sequence
  Primer
caaacaacggtagaccaatata

SEQ ID NO: 20 - DNA, 24 bp, Artificial Sequence
  Primer
aggttcagtatctatcacagtctt

SEQ ID NO: 21 - DNA, 21 bp, Artificial Sequence
  Primer
atgtccaacatggatattgac

SEQ ID NO: 22 - DNA, 21 bp, Artificial Sequence
  Primer
gctcttcctataaatcgaatg

SEQ ID NO: 23 - DNA, 21 bp, Artificial Sequence
  Primer
tgatcaagtgatcggaagtag

SEQ ID NO: 24 - DNA, 20 bp, Artificial Sequence
  Primer
gatggtctgcttaattggaa

SEQ ID NO: 25 - DNA, 20 bp, Artificial Sequence
  Primer
acagaagatggagaaggcaa

SEQ ID NO: 26 - DNA, 20 bp, Artificial Sequence
  Primer
attgtttctttggcctggat

SEQ ID NO: 27 - DNA, 20 bp, Artificial Sequence
  Primer
catcccaaaaattgccagat

SEQ ID NO: 28 - DNA, 20 bp, Artificial Sequence
  Primer
tttgggctttgccttaaatg

SEQ ID NO: 29 - DNA, 20 bp, Artificial Sequence
  Primer
acaccctcatcattgcaaca

SEQ ID NO: 30 - DNA, 20 bp, Artificial Sequence
  Primer
gcccttctgactgtggtctc

SEQ ID NO: 31 - DNA, 20 bp, Artificial Sequence
  Primer
cgacacagcagcaggaatta

SEQ ID NO: 32 - DNA, 20 bp, Artificial Sequence
  Primer
tcaaagctgcttgacactgg

SEQ ID NO: 33 - DNA, 20 bp, Artificial Sequence
  Primer
caagtgcgacattgatgacc

SEQ ID NO: 34 - DNA, 20 bp, Artificial Sequence
  Primer
taattcctgctgctgtgtcg

SEQ ID NO: 35 - DNA, 20 bp, Artificial Sequence
  Primer
gcgactgtagcacttgacga

SEQ ID NO: 36 - DNA, 20 bp, Artificial Sequence
  Primer
tcatgatcagtcccgcataa

SEQ ID NO: 37 - DNA, 20 bp, Artificial Sequence
  Primer
tgtttcaggccaatacacca

SEQ ID NO: 38 - DNA, 20 bp, Artificial Sequence
  Primer
tcatgatcagtcccgcataa

SEQ ID NO: 39 - DNA, 20 bp, Artificial Sequence
  Primer
tcatgggtaatgaagcagca

SEQ ID NO: 40 - DNA, 20 bp, Artificial Sequence
  Primer
ggagttttcccatcactgga

SEQ ID NO: 41 - DNA, 20 bp, Artificial Sequence
  Primer
tccagtgatgggaaaactcc

SEQ ID NO: 42 - DNA, 20 bp, Artificial Sequence
  Primer
tgttgagctcctttgccttt

SEQ ID NO: 43 - DNA, 20 bp, Artificial Sequence
  Primer
tggcggtataggggtaactg

SEQ ID NO: 44 - DNA, 20 bp, Artificial Sequence
  Primer
attgcggtgatggttaaagg

SEQ ID NO: 45 - DNA, 20 bp, Artificial Sequence
  Primer
ttttgccgatcccacttatc

SEQ ID NO: 46 - DNA, 20 bp, Artificial Sequence
  Primer
gcaagtctaccacggcattt

SEQ ID NO: 47 - DNA, 20 bp, Artificial Sequence
  Primer
ctccgttatcgctccatgtt

SEQ ID NO: 48 - DNA, 20 bp, Artificial Sequence
  Primer
aaggactggtcgttggtgtc

SEQ ID NO: 49 - DNA, 20 bp, Artificial Sequence
  Primer
aaatgccgtggtagatttgc

SEQ ID NO: 50 - DNA, 20 bp, Artificial Sequence
  Primer
gttgaaggggttgacgttgt

SEQ ID NO: 51 - DNA, 20 bp, Artificial Sequence
  Primer
tcctctggatggcataggac

SEQ ID NO: 52 - DNA, 20 bp, Artificial Sequence
  Primer
tgttggtgttagtgggcaaa

SEQ ID NO: 53 - DNA, 20 bp, Artificial Sequence
  Primer
acatggtcctgcaaagttcc

SEQ ID NO: 54 - DNA, 20 bp, Artificial Sequence
  Primer
gcattgtgccacgttgtatc

SEQ ID NO: 55 - DNA, 20 bp, Artificial Sequence
  Primer
cgcttcggagtacctcagtc

SEQ ID NO: 56 - DNA, 20 bp, Artificial Sequence
  Primer
ctgcatcattggtgtcaacc

SEQ ID NO: 57 - DNA, 20 bp, Artificial Sequence
  Primer
ggcaccttttacctcaacca

SEQ ID NO: 58 - DNA, 20 bp, Artificial Sequence
  Primer
tctggaccaagaaccagtcc

SEQ ID NO: 59 - DNA, 20 bp, Artificial Sequence
  Primer
ggcctaccctgctaacttcc

SEQ ID NO: 60 - DNA, 20 bp, Artificial Sequence
  Primer
ataaagaagggtgggctcgt

SEQ ID NO: 61 - DNA, 20 bp, Artificial Sequence
  Primer
atcgcagttgaatgctgttg

SEQ ID NO: 62 - DNA, 20 bp, Artificial Sequence
  Primer
gttgaaggggttgacgttgt

SEQ ID NO: 63 - DNA, 20 bp, Artificial Sequence
  Primer
acatggtcctgcaaagttcc

SEQ ID NO: 64 - DNA, 20 bp, Artificial Sequence
  Primer
gatcgaaccctgatccaaga

SEQ ID NO: 65 - DNA, 20 bp, Artificial Sequence
  Primer
aacaccaaccgaaggagatg

SEQ ID NO: 66 - DNA, 20 bp, Artificial Sequence
  Primer
cctatgccatccagaggaaa

SEQ ID NO: 67 - DNA, 20 bp, Artificial Sequence
  Primer
cagatgctcgccaactacaa

SEQ ID NO: 68 - DNA, 20 bp, Artificial Sequence
  Primer
agccatgtaacccacaaagc

SEQ ID NO: 69 - DNA, 20 bp, Artificial Sequence
  Primer
acggacgttatgtgcctttc

SEQ ID NO: 70 - DNA, 20 bp, Artificial Sequence
  Primer
gggaatattggttgcattgg

SEQ ID NO: 71 - DNA, 20 bp, Artificial Sequence
  Primer
actggttcctggtccagatg

SEQ ID NO: 72 - DNA, 20 bp, Artificial Sequence
  Primer
agccatgtaacccacaaagc

SEQ ID NO: 73 - DNA, 20 bp, Artificial Sequence
  Primer
ctggatatggccagcacttt

SEQ ID NO: 74 - DNA, 20 bp, Artificial Sequence
  Primer
cacctgaggttctggttggt

SEQ ID NO: 75 - DNA, 20 bp, Artificial Sequence
  Primer
taatgaaaagggcggacaag

SEQ ID NO: 76 - DNA, 20 bp, Artificial Sequence
  Primer
gccaatgtagtttggcctgt

SEQ ID NO: 77 - DNA, 20 bp, Artificial Sequence
  Primer
aactccgcggtagacagcta

SEQ ID NO: 78 - DNA, 20 bp, Artificial Sequence
  Primer
cgtaggtgttggtgttggtg

SEQ ID NO: 79 - DNA, 42 bp, Artificial Sequence
  Primer
tcacttgcttccgttgaggttgggctggcggtttagagttga

SEQ ID NO: 80 - DNA, 42 bp, Artificial Sequence
  Primer
ggtttcggatgttacagcgtgtgcgaccgcccttgtttatgg

SEQ ID NO: 81 - DNA, 43 bp, Artificial Sequence
  Primer
tcacttgcttccgttgagggcgttgttggcctttttcttgtct

SEQ ID NO: 82 - DNA, 44 bp, Artificial Sequence
  Primer
ggtttcggatgttacagcgtgcccggcattatttcattgttctg

SEQ ID NO: 83 - DNA, 41 bp, Artificial Sequence
  Primer
tcacttgcttccgttgaggacaaaagccgctggtggtaaag

SEQ ID NO: 84 - DNA, 44 bp, Artificial Sequence
  Primer
ggtttcggatgttacagcgtcagaaatcataacgggcaaactca

SEQ ID NO: 85 - DNA, 43 bp, Artificial Sequence
  Primer
tcacttgcttccgttgaggaagagttattgctggcgttgttgg

SEQ ID NO: 86 - DNA, 44 bp, Artificial Sequence; identical to SEQ ID NO: 82
  Primer

SEQ ID NO: 87 - DNA, 23 bp, Artificial Sequence
  Primer
ttgggctggcggtttagagttga

SEQ ID NO: 88 - DNA, 22 bp, Artificial Sequence
  Primer
gtgcgaccgcccttgtttatgg

SEQ ID NO: 89 - DNA, 24 bp, Artificial Sequence
  Primer
gcgttgttggcctttttcttgtct

SEQ ID NO: 90 - DNA, 24 bp, Artificial Sequence
  Primer
gcccggcattatttcattgttctg

SEQ ID NO: 91 - DNA, 22 bp, Artificial Sequence
  Primer
acaaaagccgctggtggtaaag

SEQ ID NO: 92 - DNA, 24 bp, Artificial Sequence
  Primer
cagaaatcataacgggcaaactca

SEQ ID NO: 93 - DNA, 24 bp, Artificial Sequence
  Primer
aagagttattgctggcgttgttgg

SEQ ID NO: 94 - DNA, 24 bp, Artificial Sequence
  Primer
gcccggcattatttcattgttctg

SEQ ID NO: 95 - DNA, 43 bp, Artificial Sequence
  Primer
tcacttgcttccgttgaggttggggtgatgggtttcagattaa

SEQ ID NO: 96 - DNA, 43 bp, Artificial Sequence
  Primer
ggtttcggatgttacagcgtctcgggaagatcgccttcttcta

SEQ ID NO: 97 - DNA, 24 bp, Artificial Sequence
  Primer
ttggggtgatgggtttcagattaa

SEQ ID NO: 98 - DNA, 23 bp, Artificial Sequence
  Primer
ctcgggaagatcgccttcttcta

SEQ ID NO: 99 - DNA, 65 bp, Artificial Sequence
  Primer
tttagagcctatgtggatggattcraaccgaacggctgcattgagggcaagctttctcaaatgtc

SEQ ID NO: 100 - DNA, 70 bp, Artificial Sequence
  Primer
acaattgaagaaagatttgaaatcactggaaccatgcgcaggcttgccgaccaaagtctcccaccgaact

SEQ ID NO: 101 - DNA, 48 bp, Artificial Sequence
  Primer
  misc_feature 7, 25, 43: n = a, c, g, or t
agcaatngaggagtgcctgattaangatccctgggttttgctnaatgc

SEQ ID NO: 102 - DNA, 70 bp, Artificial Sequence
  Primer
  misc_feature 57: n = a, c, g, or t
ccatacagccatggaacaggaacaggatacaccatggacacagtcaacagaacacancaatattcagaaa

SEQ ID NO: 103 - DNA, 53 bp, Artificial Sequence
  Primer
  misc_feature 24: n = a, c, g, or t
gggcggggagtcttcgagctctcngacgaaaaggcaacgaacccgatcgtgcc

SEQ ID NO: 104 - DNA, 59 bp, Artificial Sequence
  Primer
  misc_feature 6: n = a, c, g, or t
gatctngaggctctcatggaatggctaaagacaagaccaatcctgtcacctctgactaa

SEQ ID NO: 105 - DNA, 70 bp, Artificial Sequence
  Primer
gctgggaaatagcatggaactgatgatattcagctacaatcaagactattcgttaagtaatgaatcctca

SEQ ID NO: 106 - DNA, 70 bp, Artificial Sequence
  Primer
tctgttccagctggtttctccaattttgaaggaatgaggagctacatagacaatatagatcctaaaggag

SEQ ID NO: 107 - DNA, 70 bp, Artificial Sequence
  Primer
ttacaaccatgagctaccagaagttccatataatgcctttcttctaatgtctgatgaattggggctggcc

SEQ ID NO: 108 - DNA, 70 bp, Artificial Sequence
  Primer
acaaataagatccaaatgaaatggggaatggaagctagaagatgtctgcttcaatcaatgcaacaaatgg

SEQ ID NO: 109 - DNA, 70 bp, Artificial Sequence
  Primer
  misc_feature 24: n = a, c, g, or t
gagggaatgtattctggaatagangaatgtattagtaacaacccttgggtaatacagagtgcatactggt

SEQ ID NO: 110 - DNA, 70 bp, Artificial Sequence
  Primer
ctaccgtgttgggagtagccgcactaggtatcaaaaacattggaaacaaagaatacttatgggatggact

SEQ ID NO: 111 - DNA, 70 bp, Artificial Sequence
  Primer
ggctatgactgaaagaataaccagagacagcccaatttggttccgggatttttgtagtatagcaccggtc

SEQ ID NO: 112 - DNA, 70 bp, Artificial Sequence
  Primer
actgatcagaggaacatgattcttgaggaacaatgctacgctaagtgttgcaacctttttgaggcctgtt

SEQ ID NO: 113 - DNA, 70 bp, Artificial Sequence
  Primer
  misc_feature 14, 44: n = a, c, g, or t
aaaatccctttgtnggacatttgtctattgagggcatcaaagangcagatataaccccagcacatggtcc

SEQ ID NO: 114 - DNA, 70 bp, Artificial Sequence
  Primer
cttggaatacaagggaatacaacttaaaacaaatgctgaagacataggaaccaaaggccaaatgtgctca

SEQ ID NO: 115 - DNA, 70 bp, Artificial Sequence
  Primer
gtggcaggagcaacatcagctgagttcatagaaatgctacactgcttacaaggtgaaaattggagacaaa

SEQ ID NO: 116 - DNA, 70 bp, Artificial Sequence
  Primer
ggaacccatccccggaaagagcaaccacaagcagtgaagctgatgtcggaaggaaaacccaaaagaaaca

SEQ ID NO: 117 - DNA, 70 bp, Artificial Sequence
  Primer
ctgtttccaaagatcaaaggcactaaaaagagttggacttgacccttcattaatcagtacctttgcagga

SEQ ID NO: 118 - DNA, 70 bp, Artificial Sequence
  Primer
agagttttgtctgcattaacaggcacagaattcaagcctagatcagcattaaaatgcaagggtttccatg

SEQ ID NO: 119 - DNA, 70 bp, Artificial Sequence
  Primer
gagggacgtgatgcagatgtcaaaggaaatctactcaagatgatgaatgactcaatggctaagaaaacca

SEQ ID NO: 120 - DNA, 70 bp, Artificial Sequence
  Primer
cctatcaggaatgggaacaacagcaacaaaaaagaaaggcctgattctagctgagagaaaaatgagaaga

SEQ ID NO: 121 - DNA, 70 bp, Artificial Sequence
  Primer
gcaagtcaaaagaatggggaaggaattgcaaaggatgtaatggaagtgctaaagcagagctctatgggaa

SEQ ID NO: 122 - DNA, 70 bp, Artificial Sequence
  Primer
aaaagtgtatcacagaagtttgttcattgagtatggcaaagcattaggctcatcatctacaggcagcaaa

SEQ ID NO: 123 - DNA, 70 bp, Artificial Sequence
  Primer
gaaagtctatttgttaatatattcatgcaagcttatggagccggtcaaacaatgctaaggtggggggtca

SEQ ID NO: 124 - DNA, 70 bp, Artificial Sequence
  Primer
acgctgttgtgtggagaaattctgtatgctaaacatgctgattacaaatatgctgcagaaataggaatac

SEQ ID NO: 125 - DNA, 70 bp, Artificial Sequence
  Primer
ttaaggaatcatcaggtaatatcccacaaaatcagaggccctcagcaccagacacacccataatcttatt

SEQ ID NO: 126 - DNA, 70 bp, Artificial Sequence
  Primer
tgagcaatcaaaggagtgcaacatcaacatatccactacaaattacccatgcaaagtcagcacaggaaga

SEQ ID NO: 127 - DNA, 70 bp, Artificial Sequence
  Primer
ctgttccattggcagcaacagagtagggatcatcaagcagctgaacaaaggttgctcctatataaccaac

SEQ ID NO: 128 - DNA, 70 bp, Artificial Sequence
  Primer
acttaatgacagatgctgaactagccagggccgtttctaacatgccgacatctgcaggacaaataaaatt

SEQ ID NO: 129 - DNA, 70 bp, Artificial Sequence
  Primer
aaaaaaagggaaactatgcttgcctcttaagagaagaccaagggtggtattgtcagaatgcagggtcaac

SEQ ID NO: 130 - DNA, 70 bp, Artificial Sequence
  Primer
gaaaagaacacaccagttacaataccagcatttatcaaatcggtttctatcaaagagagtgaatcagcca

SEQ ID NO: 131 - DNA, 70 bp, Artificial Sequence
  Primer
caaatcagttggcaaaaaaacacatgatctgatcgcattatgtgattttatggatctagaaaagaacaca

SEQ ID NO: 132 - DNA, 70 bp, Artificial Sequence
  Primer
cagctaaagacactgactataactactctgtatgctgcatcacaaagtggtccaatactaaaagtgaatg

SEQ ID NO: 133 - DNA, 70 bp, Artificial Sequence
  Primer
aaaagaacacaccagttacaataccagcatttatcaaatcggtttctatcaaagagagtgaatcagccac

SEQ ID NO: 134 - DNA, 70 bp, Artificial Sequence
  Primer
ctattataggagaaaaagtgaacactgtatctgaaacattggaattacctactatcagtagacccaccaa

SEQ ID NO: 135 - DNA, 70 bp, Artificial Sequence
  Primer
aagttagcatggacagacaaaggtggggcaatcaaaactgaagcaaagcaaacaatcaaagttatggatc

SEQ ID NO: 136 - DNA, 70 bp, Artificial Sequence
  Primer
caggaaaatacacaaagttggagaaagatgctctagacttgctttcagacaatgaagaagaagatgcaga

SEQ ID NO: 137 - DNA, 70 bp, Artificial Sequence
  Primer
ctaatagcagacataataaaagaagccaagggaaaagcagcagaaatgatggaagaagaaatgaaccagc

SEQ ID NO: 138 - DNA, 70 bp, Artificial Sequence
  Primer
ctgacacctaccaaggtataaaatcaaacggaaacggtaatcctcaaaactggaccaaaaatgacgattt

SEQ ID NO: 139 - DNA, 70 bp, Artificial Sequence
  Primer
tcctctactccaacattgcactgtacctgcctgacaagctaaaatacactcctacaaatgtggaaatatc

SEQ ID NO: 140 - DNA, 70 bp, Artificial Sequence
  Primer
gctatcggaggcagagtactaaaaaagactactcccatgaaaccatgctacggatcgtatgccagaccta

SEQ ID NO: 141 - DNA, 70 bp, Artificial Sequence
  Primer
agtattgttttgtacagtgaggatgttaatatggaaactcctgatactcacatttcatacaaaccaagca

SEQ ID NO: 142 - DNA, 70 bp, Artificial Sequence
  Primer
gggaaacgatcttagagttgacggggctagcattaagtttgacagcatttgtctttacgccaccttcttc

SEQ ID NO: 143 - DNA, 70 bp, Artificial Sequence
  Primer
ttgccattaaaaacctcctcctcctgccaggctcatatacatatgaatggaacttcaggaaggatgttaa

SEQ ID NO: 144 - DNA, 70 bp, Artificial Sequence
  Primer
ttgcaacacgtaatgaaataggagtgggtaacaactttgccatggaaattaacctaaatgccaacctatg

SEQ ID NO: 145 - DNA, 70 bp, Artificial Sequence
  Primer
ttggggtaactgacacctatcaagctattaaggctaatggcaatggctcaggcgataatggagatattac

SEQ ID NO: 146 - DNA, 70 bp, Artificial Sequence
  Primer
aggtatcaaggcattaaagttaaaaccgatgacgctaatggatgggaaaaagatgctaatgttgatacag

SEQ ID NO: 147 - DNA, 70 bp, Artificial Sequence
  Primer
gagaagttttctgtactccaatgtggctttgtaccttccagatgtttacaagtacacgccacctaacatt

SEQ ID NO: 148 - DNA, 70 bp, Artificial Sequence
  Primer
atcagtcatttaacgactacctctctgcagctaacatgctttaccccattcctgccaatgcaaccaacat

SEQ ID NO: 149 - DNA, 70 bp, Artificial Sequence
  Primer
ctacttcgtatattctggatctattccctacctggatggcaccttttaccttaaccacactttcaagaag

SEQ ID NO: 150 - DNA, 70 bp, Artificial Sequence
  Primer
acctgccagtggaaggatgctaacagcaaaatgcatacctttggggtagctgccatgccaggtgttactg

SEQ ID NO: 151 - DNA, 70 bp, Artificial Sequence
  Primer
atagaagctgatgggctgcctattagaatagattcaacttctggaactgacacagtaatttatgctgata

SEQ ID NO: 152 - DNA, 70 bp, Artificial Sequence
  Primer
ttgaaattaagcgcaccgtggacggcgaggggtacaacgtggcccagtgcaacatgaccaaggactggtt

SEQ ID NO: 153 - DNA, 70 bp, Artificial Sequence
  Primer
cggcaacgaccggctcctgacgcccaacgagtttgaaattaagcgcaccgtggacggcgaggggtacaac

SEQ ID NO: 154 - DNA, 70 bp, Artificial Sequence
  Primer
ctccagtaactttatgtccatgggcgcactcacagacctgggccaaaaccttctctacgccaactccgcc

SEQ ID NO: 155 - DNA, 70 bp, Artificial Sequence
  Primer
gctaacttcccctatccgcttataggcaagaccgcagttgacagcattacccagaaaaagtttctttgcg

SEQ ID NO: 156 - DNA, 70 bp, Artificial Sequence
  Primer
acagtccttccaacgtaaaaatttctgataacccaaacacctacgactacatgaacaagcgagtggtggc

SEQ ID NO: 157 - DNA, 70 bp, Artificial Sequence
  Primer
aagatgaacttccaaattactgctttccactgggaggtgtgattaatacagagactcttaccaaggtaaa

SEQ ID NO: 158 - DNA, 70 bp, Artificial Sequence
  Primer
agctaacatgctttaccccatccctgccaatgcaaccaacattccaatttccatcccatctcgcaactgg

SEQ ID NO: 159 - DNA, 70 bp, Artificial Sequence
  Primer
ttcaactcttgaagccatgctgcgcaacgataccaatgatcagtcattcaacgactacctctctgcagct

SEQ ID NO: 160 - DNA, 70 bp, Artificial Sequence
  Primer
aggctgtggacagctatgatcccgatgttcgtattattgaaaatcatggcgtcgaggatgaactgcctaa

SEQ ID NO: 161 - DNA, 70 bp, Artificial Sequence
  Primer
tgaaattgtgctttacacggaaaatgtcaatttggaaactccagacagccatgtggtatacaagccagga

SEQ ID NO: 162 - DNA, 70 bp, Artificial Sequence
  Primer
catcggctatcagggcttctacattccagaaggatacaaagatcgcatgtattcatttttcagaaacttc

SEQ ID NO: 163 - DNA, 70 bp, Artificial Sequence
  Primer
gctgcttctcccaggctcctacacttatgagtggaactttaggaaggatgtgaacatggttctacagagt

SEQ ID NO: 164 - DNA, 70 bp, Artificial Sequence
  Primer
atgacaccaatgatcagtcattcaacgactacctatctgcagctaacatgctctaccccattcctgccaa

SEQ ID NO: 165 - DNA, 70 bp, Artificial Sequence
  Primer
cttgccaactacaacattggataccagggcttctacgttcctgagggttacaaggatcgcatgtactcct

SEQ ID NO: 166 - DNA, 70 bp, Artificial Sequence
  Primer
gatcgcatgtactccttcttcagaaacttccagcccatgagtagacaggtggttgatgagattaactaca

SEQ ID NO: 167 - DNA, 70 bp, Artificial Sequence
  Primer
cccctaagggcgctcccaatacatctcagtggattgctgaaggcgtaaaaaaagaagatgggggatctga

SEQ ID NO: 168 - DNA, 70 bp, Artificial Sequence
  Primer
agaaaatgtaaatttggaaactccagattcccatgttgtttacaaagcaggaacttcagacgaaagctct

SEQ ID NO: 169 - DNA, 70 bp, Artificial Sequence
  Primer
tgtggctaccaatactgtttaccaaggtgttaagttacaaactggtcaaactgacaaatggcagaaagat

SEQ ID NO: 170 - DNA, 70 bp, Artificial Sequence
  Primer
ccgaattgggaagggtagcgtattcgccatggaaatcaatctccaggccaacctgtggaagagttttctg

SEQ ID NO: 171 - DNA, 70 bp, Artificial Sequence
  Primer
ttgatgaggtcaattacaaagacttcaaggccgtcgccataccctaccaacacaacaactctggctttgt

SEQ ID NO: 172 - DNA, 70 bp, Artificial Sequence
  Primer
tgacgaagaggaagagaaaaatctcaccacttacacttttggaaatgccccagtgaaagcagaaggtggt

SEQ ID NO: 173 - DNA, 70 bp, Artificial Sequence
  Primer
agaagattttgacattgacatggctttctttgattccaacactattaacacaccagatgttgtgctgtat

SEQ ID NO: 174 - DNA, 70 bp, Artificial Sequence
  Primer
aatggggttatgttggttcactctccactaatcaccatgcaatttgtaatgttcatagaaatgagcatgt

SEQ ID NO: 175 - DNA, 70 bp, Artificial Sequence
  Primer
gtgtatgactgctttgttaagaatgtggattggtcaattacctaccctatgatagctaatgaaaatgcca

SEQ ID NO: 176 - DNA, 70 bp, Artificial Sequence
  Primer
ttgcatcttcttttgttggtatgccatcttttgttgcatatgaaacagcaagacaagagtatgaaaatgc

SEQ ID NO: 177 - DNA, 70 bp, Artificial Sequence
  Primer
aaatggttcctcaccacaaataatcaaacaattgaagaaggctatgaatgttgcaaaagctgagtttgac

SEQ ID NO: 178 - DNA, 70 bp, Artificial Sequence
  Primer
ctgctgcagctatgtacaaagaagcacgtgctgttaatagaaaatcaaaagttgttagtgccatgcatag

SEQ ID NO: 179 - DNA, 70 bp, Artificial Sequence
  Primer
acgtttggacatgtctagtgttgacactatccttaatatggcacgtaatggtgttgtccctctttccgtt

SEQ ID NO: 180 - DNA, 70 bp, Artificial Sequence
  Primer
ctggtggtaaagtttcattttctgatgacgttgaagtaaaagacattgaacctgtttacagagtcaagct

SEQ ID NO: 181 - DNA, 70 bp, Artificial Sequence
  Primer
tttacagagtcaagctttgctttgagtttgaagatgaaaaacttgtagatgtttgtgaaaaggcaattgg

SEQ ID NO: 182 - DNA, 70 bp, Artificial Sequence
  Primer
gatgtttgtgaaaaggcaattggcaagaaaattaaacatgaaggtgactgggatagcttttgtaagacta

SEQ ID NO: 183 - DNA, 70 bp, Artificial Sequence
  Primer
gcgttgttggcctttttcttgtctaagcatagtgattttggtcttggtgatcttgtcgattcttattttg

SEQ ID NO: 184 - DNA, 70 bp, Artificial Sequence
  Primer
agcaagacaagagtatgaaaatgctgttgcaaatggttcctcaccacaaataatcaaacaattgaagaag

SEQ ID NO: 185 - DNA, 70 bp, Artificial Sequence
  Primer
ttgaagaaggctatgaatgttgcaaaagctgagtttgacagggaatcatctgttcaaaagaaaattaaca

SEQ ID NO: 186 - DNA, 70 bp, Artificial Sequence; identical to SEQ ID NO: 178
  Primer

SEQ ID NO: 187 - DNA, 70 bp, Artificial Sequence
  Primer
ctcacatcctaggaagatgcatagttttagatgttaaaggtgtagaagaattgcatgacgatttagttaa

SEQ ID NO: 188 - DNA, 70 bp, Artificial Sequence
  Primer
ggattggccattgcaccatagctcaactcacggatgcagcactgtccattaaggaaaatgttgattttat

SEQ ID NO: 189 - DNA, 70 bp, Artificial Sequence
  Primer
gcatgcaattcaattataaaatcaccatcaacccctcatcaccggctagacttgaaatagttaagctcgg

SEQ ID NO: 190 - DNA, 70 bp, Artificial Sequence
  Primer
atagttagtcactggatgggaattcgttttgaatacacatcacccactgataagctagctatgattatgg

SEQ ID NO: 191 - DNA, 56 bp, Artificial Sequence
  Primer
ttaccctaatatgtttatcacccgcgaagaagctattcgtcacgttcgtgcgtgga

SEQ ID NO: 192 - DNA, 56 bp, Artificial Sequence
  Primer
ctgacaagtatgtccgcaatctacaacacaggctctatgagtgtctctatagaaat

SEQ ID NO: 193 - DNA, 56 bp, Artificial Sequence
  Primer
cataacacttgctgtaacttatcacaccgtttctacaggttagctaacgagtgtgc

SEQ ID NO: 194 - DNA, 50 bp, Artificial Sequence
  Primer
ttaccctaatatgtttatcacccgcgaagaagctattcgtcacgttcgtg

SEQ ID NO: 195 - DNA, 70 bp, Artificial Sequence
  Primer
gcgttctcttaaagctcctgccgtagtgtcagtatcatcaccagatgctgttactacatataatggatac

SEQ ID NO: 196 - DNA, 70 bp, Artificial Sequence
  Primer
ctttggctggctcttacagagattggtcctattcaggacagcgtacagagttaggtgttgaatttcttaa

SEQ ID NO: 197 - DNA, 70 bp, Artificial Sequence
  Primer
ctacgtagtgaagctttcgagtactaccatactcttgatgagagttttcttggtaggtacatgtctgctt

SEQ ID NO: 198 - DNA, 70 bp, Artificial Sequence
  Primer
tgccaattggttatgtgacacatggttttaatcttgaagaggctgcgcgctgtatgcgttctcttaaagc

SEQ ID NO: 199 - DNA, 70 bp, Artificial Sequence
  Primer
tataaagttaccaagggaaagcccgtaaaaggtgcttggaacattggacaacagagatcagttttaacac

SEQ ID NO: 200 - DNA, 69 bp, Artificial Sequence
  Primer
tgcttcattgatgttgttaacaaggcactcgaaatgtgcattgatcaagtcactatcgctggcgcaaag

SEQ ID NO: 201 - DNA, 70 bp, Artificial Sequence
  Primer
tgtcgacgccatggtttatacttcagacctgctcaccaacagtgtcattattatggcatatgtaactggt

SEQ ID NO: 202 - DNA, 70 bp, Artificial Sequence
  Primer
tactgttgaaaaactcaggcctatctttgaatggattgaggcgaaacttagtgcaggagttgaatttctc

SEQ ID NO: 203 - DNA, 61 bp, Artificial Sequence
  Primer
acctattctgttgcttgaccaagctcttgtatcagacgttggagatagtactgaagtttcc

SEQ ID NO: 204 - DNA, 70 bp, Artificial Sequence
  Primer
gcctattaatgtcatagtttttgatggcaagtccaaatgcgacgagtctgcttctaagtctgcttctgtg

SEQ ID NO: 205 - DNA, 70 bp, Artificial Sequence
  Primer
tgagagctaacaacactaaaggttcactgcctattaatgtcatagtttttgatggcaagtccaaatgcga

SEQ ID NO: 206 - DNA, 70 bp, Artificial Sequence
  Primer
acttgcatgatgtgctataagcgcaatcgtgccacacgcgttgagtgtacaactattgttaatggcatga

SEQ ID NO: 207 - DNA, 70 bp, Artificial Sequence
  Primer
ggcgatgtagtggctattgactatagacactattcagcgagtttcaagaaaggtgctaaattactgcata

SEQ ID NO: 208 - DNA, 70 bp, Artificial Sequence
  Primer
tcaaaccaaacacttggtgtttacgttgtctttggagtacaaagccagtagatacttcaaattcatttga

SEQ ID NO: 209 - DNA, 63 bp, Artificial Sequence
  Primer
tagtgctgttggcaacatttgctacacaccttccaaactcattgagtatagtgattttgctac

SEQ ID NO: 210 - DNA, 70 bp, Artificial Sequence
  Primer
tcatagctaacatctttactcctcttgtgcaacctgtgggtgctttagatgtgtctgcttcagtagtggc

SEQ ID NO: 211 - DNA, 70 bp, Artificial Sequence
  Primer
ggtattattgccatattggtgacttgtgctgcctactactttatgaaattcagacgtgtttttggtgagt

SEQ ID NO: 212 - DNA, 70 bp, Artificial Sequence
  Primer
gtgatgtcagagaaactatgacccatcttctacagcatgctaatttggaatctgcaaagcgagttcttaa

SEQ ID NO: 213 - DNA, 70 bp, Artificial Sequence
  Primer
aaccatcaagcctgtgtcgtataaactcgatggagttacttacacagagattgaaccaaaattggatggg

SEQ ID NO: 214 - DNA, 70 bp, Artificial Sequence
  Primer
gttttctacaaggaaacatcttacactacaaccatcaagcctgtgtcgtataaactcgatggagttactt

SEQ ID NO: 215 - DNA, 70 bp, Artificial Sequence
  Primer
ccttgaatgaggatctccttgagatactgagtcgtgaacgtgttaacattaacattgttggcgattttca

SEQ ID NO: 216 - DNA, 70 bp, Artificial Sequence
  Primer
gccatggtttatacttcagacctgctcaccaacagtgtcattattatggcatatgtaactggtggtcttg

SEQ ID NO: 217 - DNA, 70 bp, Artificial Sequence
  Primer
caacagacttctcagtggttgtctaatcttttgggcactactgttgaaaaactcaggcctatctttgaat

SEQ ID NO: 218 - DNA, 56 bp, Artificial Sequence
  Primer
ttcccgtcaggcaaagttgaagggtgcatggtacaagtaacctgtggaactacaac

SEQ ID NO: 219 - DNA, 56 bp, Artificial Sequence
  Primer
ggttcaccatctggtgtttatcagtgtgccatgagacctaatcataccattaaagg

SEQ ID NO: 220 - DNA, 52 bp, Artificial Sequence
  Primer
agatcatgttgacatattgggacctctttctgctcaaacaggaattgccgtc

SEQ ID NO: 221 - DNA, 70 bp, Artificial Sequence
  Primer
taaaaaggacaaaaagaaaaagactgatgaagctcagcctttgccgcagagacaaaagaagcagcccact

SEQ ID NO: 222 - DNA, 70 bp, Artificial Sequence
  Primer
acggcaaaatgaaagagctcagccccagatggtacttctattacctaggaactggcccagaagcttcact

SEQ ID NO: 223 - DNA, 70 bp, Artificial Sequence
  Primer
ggcgctaacaaagaaggcatcgtatgggttgcaactgagggagccttgaatacacccaaagaccacattg

SEQ ID NO: 224 - DNA, 70 bp, Artificial Sequence
  Primer
gtccagatgaccaaattggctactaccgaagagctacccgacgagttcgtggtggtgacggcaaaatgaa

SEQ ID NO: 225 - DNA, 70 bp, Artificial Sequence
  Primer
gaggtggtgaaactgccctcgcgctattgctgctagacagattgaaccagcttgagagcaaagtttctgg

SEQ ID NO: 226 - DNA, 70 bp, Artificial Sequence
  Primer
aaaagaaaaagactgatgaagctcagcctttgccgcagagacaaaagaagcagcccactgtgactcttct

SEQ ID NO: 227 - DNA, 70 bp, Artificial Sequence
  Primer
aaattgcacaatttgctccaagtgcctctgcattctttggaatgtcacgcattggcatggaagtcacacc

SEQ ID NO: 228 - DNA, 70 bp, Artificial Sequence
  Primer
accaatttaacaaggcgattagtcaaattcaagaatcacttacaacaacatcaactgcattgggcaagct

SEQ ID NO: 229 - DNA, 70 bp, Artificial Sequence
  Primer
cacctggaacaaatgcttcatctgaagttgctgttctatatcaagatgttaactgcactgatgtttctac

SEQ ID NO: 230 - DNA, 70 bp, Artificial Sequence
  Primer
aaagggctaccaccttatgtccttcccacaagcagccccgcatggtgttgtcttcctacatgtcacgtat

SEQ ID NO: 231 - DNA, 70 bp, Artificial Sequence
  Primer
tcaggaaattgtgatgtcgttattggcatcattaacaacacagtttatgatcctctgcaacctgagcttg

SEQ ID NO: 232 - DNA, 70 bp, Artificial Sequence
  Primer
ttgatcttggcgacatttcaggcattaacgcttctgtcgtcaacattcaaaaagaaattgaccgcctcaa

SEQ ID NO: 233 - DNA, 70 bp, Artificial Sequence
  Primer
gaggaacttcaccacagcgccagcaatttgtcatgaaggcaaagcatacttccctcgtgaaggtgttttt

SEQ ID NO: 234 - DNA, 65 bp, Artificial Sequence; identical to SEQ ID NO: 99
  Primer

SEQ ID NO: 235 - DNA, 70 bp, Artificial Sequence; identical to SEQ ID NO: 100
  Primer

SEQ ID NO: 236 - DNA, 48 bp, Artificial Sequence; identical to SEQ ID NO: 101
  Primer
  misc_feature 7, 25, 43: n = a, c, g, or t

SEQ ID NO: 237 - DNA, 70 bp, Artificial Sequence; identical to SEQ ID NO: 102
  Primer
  misc_feature 57: n = a, c, g, or t

SEQ ID NO: 238 - DNA, 53 bp, Artificial Sequence; identical to SEQ ID NO: 103
  Primer
  misc_feature 24: n = a, c, g, or t

SEQ ID NO: 239 - DNA, 59 bp, Artificial Sequence; identical to SEQ ID NO: 104
  Primer
  misc_feature 6: n = a, c, g, or t

SEQ ID NO: 240 - DNA, 70 bp, Artificial Sequence; identical to SEQ ID NO: 105
  Primer

SEQ ID NO: 241 - DNA, 70 bp, Artificial Sequence; identical to SEQ ID NO: 106
  Primer

SEQ ID NO: 242 - DNA, 70 bp, Artificial Sequence; identical to SEQ ID NO: 107
  Primer

SEQ ID NO: 243 - DNA, 70 bp, Artificial Sequence; identical to SEQ ID NO: 108
  Primer

SEQ ID NO: 244 - DNA, 70 bp, Artificial Sequence; identical to SEQ ID NO: 109
  Primer
  misc_feature 24: n = a, c, g, or t

SEQ ID NO: 245 - DNA, 70 bp, Artificial Sequence; identical to SEQ ID NO: 110
  Primer

SEQ ID NO: 246 - DNA, 70 bp, Artificial Sequence; identical to SEQ ID NO: 111
  Primer

SEQ ID NO: 247 - DNA, 70 bp, Artificial Sequence; identical to SEQ ID NO: 112
  Primer

SEQ ID NO: 248 - DNA, 70 bp, Artificial Sequence; identical to SEQ ID NO: 113
  Primer
  misc_feature 14, 44: n = a, c, g, or t

SEQ ID NO: 249 - DNA, 70 bp, Artificial Sequence; identical to SEQ ID NO: 114
  Primer

SEQ ID NO: 250 - DNA, 70 bp, Artificial Sequence; identical to SEQ ID NO: 115
  Primer

SEQ ID NO: 251 - DNA, 70 bp, Artificial Sequence; identical to SEQ ID NO: 116
  Primer

SEQ ID NO: 252 - DNA, 70 bp, Artificial Sequence; identical to SEQ ID NO: 117
  Primer

SEQ ID NO: 253 - DNA, 70 bp, Artificial Sequence; identical to SEQ ID NO: 118
  Primer

SEQ ID NO: 254 - DNA, 70 bp, Artificial Sequence; identical to SEQ ID NO: 119
  Primer

SEQ ID NO: 255 - DNA, 70 bp, Artificial Sequence; identical to SEQ ID NO: 120
  Primer

SEQ ID NO: 256 - DNA, 70 bp, Artificial Sequence; identical to SEQ ID NO: 121
  Primer

SEQ ID NO: 257 - DNA, 70 bp, Artificial Sequence; identical to SEQ ID NO: 138
  Primer

SEQ ID NO: 258 - DNA, 70 bp, Artificial Sequence; identical to SEQ ID NO: 139
  Primer

SEQ ID NO: 259 - DNA, 70 bp, Artificial Sequence; identical to SEQ ID NO: 140
  Primer

SEQ ID NO: 260 - DNA, 70 bp, Artificial Sequence; identical to SEQ ID NO: 141
  Primer

SEQ ID NO: 261 - DNA, 70 bp, Artificial Sequence; identical to SEQ ID NO: 142
  Primer

SEQ ID NO: 262 - DNA, 70 bp, Artificial Sequence; identical to SEQ ID NO: 143
  Primer

SEQ ID NO: 263 - DNA, 70 bp, Artificial Sequence; identical to SEQ ID NO: 144
  Primer

SEQ ID NO: 264 - DNA, 70 bp, Artificial Sequence; identical to SEQ ID NO: 145
  Primer

SEQ ID NO: 265 - DNA, 70 bp, Artificial Sequence; identical to SEQ ID NO: 146
  Primer

SEQ ID NO: 266 - DNA, 70 bp, Artificial Sequence; identical to SEQ ID NO: 147
  Primer

SEQ ID NO: 267 - DNA, 70 bp, Artificial Sequence; identical to SEQ ID NO: 148
  Primer

SEQ ID NO: 268 - DNA, 70 bp, Artificial Sequence; identical to SEQ ID NO: 149
  Primer

SEQ ID NO: 269 - DNA, 70 bp, Artificial Sequence; identical to SEQ ID NO: 150
  Primer

SEQ ID NO: 270 - DNA, 70 bp, Artificial Sequence; identical to SEQ ID NO: 151
  Primer

SEQ ID NO: 271 - DNA, 70 bp, Artificial Sequence; identical to SEQ ID NO: 152
  Primer

SEQ ID NO: 272 - DNA, 70 bp, Artificial Sequence; identical to SEQ ID NO: 153
  Primer

SEQ ID NO: 273 - DNA, 70 bp, Artificial Sequence; identical to SEQ ID NO: 154
  Primer

SEQ ID NO: 274 - DNA, 70 bp, Artificial Sequence; identical to SEQ ID NO: 155
  Primer

SEQ ID NO: 275 - DNA, 70 bp, Artificial Sequence; identical to SEQ ID NO: 156
  Primer

SEQ ID NO: 276 - DNA, 70 bp, Artificial Sequence; identical to SEQ ID NO: 157
  Primer

SEQ ID NO: 277 - DNA, 70 bp, Artificial Sequence; identical to SEQ ID NO: 158
  Primer

SEQ ID NO: 278 - DNA, 70 bp, Artificial Sequence; identical to SEQ ID NO: 159
  Primer

SEQ ID NO: 279 - DNA, 70 bp, Artificial Sequence; identical to SEQ ID NO: 160
  Primer

SEQ ID NO: 280 - DNA, 70 bp, Artificial Sequence; identical to SEQ ID NO: 161
  Primer

SEQ ID NO: 281 - DNA, 70 bp, Artificial Sequence; identical to SEQ ID NO: 162
  Primer

SEQ ID NO: 282 - DNA, 70 bp, Artificial Sequence; identical to SEQ ID NO: 163
  Primer

SEQ ID NO: 283 - DNA, 70 bp, Artificial Sequence; identical to SEQ ID NO: 164
  Primer

SEQ ID NO: 284 - DNA, 70 bp, Artificial Sequence; identical to SEQ ID NO: 165
  Primer

SEQ ID NO: 285 - DNA, 70 bp, Artificial Sequence; identical to SEQ ID NO: 166
  Primer

SEQ ID NO: 286 - DNA, 70 bp, Artificial Sequence; identical to SEQ ID NO: 167
  Primer

SEQ ID NO: 287 - DNA, 70 bp, Artificial Sequence; identical to SEQ ID NO: 168
  Primer

SEQ ID NO: 288 - DNA, 70 bp, Artificial Sequence; identical to SEQ ID NO: 169
  Primer

SEQ ID NO: 289 - DNA, 70 bp, Artificial Sequence; identical to SEQ ID NO: 170
  Primer

SEQ ID NO: 290 - DNA, 70 bp, Artificial Sequence; identical to SEQ ID NO: 171
  Primer

SEQ ID NO: 291 - DNA, 70 bp, Artificial Sequence; identical to SEQ ID NO: 172
  Primer

SEQ ID NO: 292 - DNA, 70 bp, Artificial Sequence; identical to SEQ ID NO: 173
  Primer

SEQ ID NO: 293 - DNA, 70 bp, Artificial Sequence; identical to SEQ ID NO: 187
  Primer

SEQ ID NO: 294 - DNA, 70 bp, Artificial Sequence; identical to SEQ ID NO: 188
  Primer

SEQ ID NO: 295 - DNA, 70 bp, Artificial Sequence; identical to SEQ ID NO: 189
  Primer

SEQ ID NO: 296 - DNA, 70 bp, Artificial Sequence; identical to SEQ ID NO: 190
  Primer

SEQ ID NO: 297 - DNA, 70 bp, Artificial Sequence; identical to SEQ ID NO: 174
  Primer

SEQ ID NO: 298 - DNA, 70 bp, Artificial Sequence; identical to SEQ ID NO: 175
  Primer

SEQ ID NO: 299 - DNA, 70 bp, Artificial Sequence; identical to SEQ ID NO: 176
  Primer

SEQ ID NO: 300 - DNA, 70 bp, Artificial Sequence; identical to SEQ ID NO: 177
  Primer

SEQ ID NO: 301 - DNA, 70 bp, Artificial Sequence; identical to SEQ ID NO: 178
  Primer

SEQ ID NO: 302 - DNA, 70 bp, Artificial Sequence; identical to SEQ ID NO: 179
  Primer

SEQ ID NO: 303 - DNA, 70 bp, Artificial Sequence; identical to SEQ ID NO: 180
  Primer

SEQ ID NO: 304 - DNA, 70 bp, Artificial Sequence; identical to SEQ ID NO: 181
  Primer

SEQ ID NO: 305 - DNA, 70 bp, Artificial Sequence; identical to SEQ ID NO: 182
  Primer

SEQ ID NO: 306 - DNA, 70 bp, Artificial Sequence; identical to SEQ ID NO: 183
  Primer

SEQ ID NO: 307 - DNA, 70 bp, Artificial Sequence; identical to SEQ ID NO: 184
  Primer

SEQ ID NO: 308 - DNA, 70 bp, Artificial Sequence; identical to SEQ ID NO: 185
  Primer

SEQ ID NO: 309 - DNA, 70 bp, Artificial Sequence; identical to SEQ ID NO: 178
  Primer

SEQ ID NO: 310 - DNA, 70 bp, Artificial Sequence
  Primer
cgggataaggcactctctatcagaatggatgtcttgctgctataatagatagagaaggttatagcagact

SEQ ID NO: 311 - DNA, 70 bp, Artificial Sequence
  Primer
ccctcgcaggaaagtcgggataaggcactctctatcagaatggatgtcttgctgctataatagatagaga

SEQ ID NO: 312 - DNA, 68 bp, Artificial Sequence
  Primer
atggatgtttgaggacgcagaggagaagttggacaaccctagtagttcagaggtggatatagtatgct

SEQ ID NO: 313 - DNA, 68 bp, Artificial Sequence
  Primer
ccttgggttatgtacttgcgtaagtgtggcgaaaagggtgcctacaataaagatcataaacgtgtcgg

SEQ ID NO: 314 - DNA, 68 bp, Artificial Sequence
  Primer
ggggatgctggttttactagcatactcagtggtttgttatatgattcaccctgtttttcacagcaagg

SEQ ID NO: 315 - DNA, 68 bp, Artificial Sequence
  Primer
catgacggcagttgcttgtcaacccccgtactgttattttcgtaattctactaccaactatgttggtg

SEQ ID NO: 316 - DNA, 70 bp, Artificial Sequence
  Primer
ggctgagtgattacatcacaggtttgggtagagcttttggtgtcgggttcactgaccaaatctcaacaaa

SEQ ID NO: 317 - DNA, 70 bp, Artificial Sequence
  Primer
gaaaagctattagcttggtagacagaactaccaacgttaggtatagtgtggatcaactggtcacggctat

SEQ ID NO: 318 - DNA, 70 bp, Artificial Sequence
  Primer
ggccaagtaatagctagacataaggttagggagtttaacataaatccagtcaacacggcaactaagtcaa

SEQ ID NO: 319 - DNA, 70 bp, Artificial Sequence
  Primer
gataacaagggcatgttattcaccagtaattttgttctagcctccacaaattctaacacactaagccccc

SEQ ID NO: 320 - DNA, 70 bp, Artificial Sequence
  Primer
ggccaagaagtaaggttgtgtttagtaccactcagggtttaccagttatgttaacacctggatctgggca

SEQ ID NO: 321 - DNA, 70 bp, Artificial Sequence
  Primer
gtaatgcgtaagtgcgggatgggaccaactactttgggtgtccgtgtttcctgtttttcttttgattgca

SEQ ID NO: 322 - DNA, 70 bp, Artificial Sequence
  Primer
taaaagaggattcagagctgatgagcgccactctttccttatacaccctacctttcctgtggctgagatt

SEQ ID NO: 323 - DNA, 70 bp, Artificial Sequence
  Primer
gcaagtttcatcagggtttattaatagttgccgccatcccagaacatcaattggcatctgcaacaagtgg

SEQ ID NO: 324 - DNA, 70 bp, Artificial Sequence
  Primer
atatatgaaggaacaccagtggcgaaggcgaaaacttaggccattactgacgcttaggcttgaaagtgtg

SEQ ID NO: 325 - DNA, 70 bp, Artificial Sequence
  Primer
gcagtagggaatttttcacaatgagcgaaagcttgatggagcaatgccgcgtgaacgatgaaggtcttta

SEQ ID NO: 326 - DNA, 70 bp, Artificial Sequence
  Primer
aacacattaagtatctcgcctgggtagtacattcgcaagaatgaaactcaaacggaattgacggggaccc

SEQ ID NO: 327 - DNA, 70 bp, Artificial Sequence
  Primer
acaccgtaaacgatagatactagctgtcggggcgatcccctcggtagtgaagttaacacattaagtatct

SEQ ID NO: 328 - DNA, 70 bp, Artificial Sequence
  Primer
acatccttggcaaagttatggaaacataatggaggttaaccgagtgacaggtggtgcatggttgtcgtca

SEQ ID NO: 329 - DNA, 70 bp, Artificial Sequence
  Primer
ttataacttaaccgtcggcagttgggtaagagaccacgtccgatcaattgtcgagggcgcgtgggaagtg

SEQ ID NO: 330 - DNA, 70 bp, Artificial Sequence
  Primer
atacccagacctgtgttcacgcagatgcaggtcagtgatcacccagcactccacgcaatttcgcggtata

SEQ ID NO: 331 - DNA, 70 bp, Artificial Sequence
  Primer
agaaactcctagatgaggttcttgcccccggtgggccttataacttaaccgtcggcagttgggtaagaga

SEQ ID NO: 332 - DNA, 70 bp, Artificial Sequence; identical to SEQ ID NO: 330
  Primer

SEQ ID NO: 333 - DNA, 66 bp, Artificial Sequence
  Primer
tcttacttcaaccctggcggcagctactacaagcagtaccaccctaccgcgtgcgaggttgaacct

SEQ ID NO: 334 - DNA, 70 bp, Artificial Sequence
  Primer
aaggcttgtttcagagattgcaatgcatactactgaggacaggatcagtagagcagttggacccagacaa

SEQ ID NO: 335 - DNA, 70 bp, Artificial Sequence
  Primer
aggatcagtagagcagttggacccagacaagcccaagtgtcattcctacacggtgatcaaagtgagaatg

SEQ ID NO: 336 - DNA, 66 bp, Artificial Sequence
  Primer
tcagtagagcagttggacccagacaagcccaagtgtcattcctacacggtgatcaaagtgagaatg

SEQ ID NO: 337 - DNA, 70 bp, Artificial Sequence
  Primer
cccagggaatgtacgggggaacttacctagttgaaaagcctaatctgagcagcaaaggatcagaattatc

SEQ ID NO: 338 - DNA, 70 bp, Artificial Sequence; identical to SEQ ID NO: 337
  Primer

SEQ ID NO: 339 - DNA, 70 bp, Artificial Sequence
  Primer
caaacccacaaacaaaccaaccaccaaaaccacaaacaaaagagacccaaaaacaccagccaaaacgacg

SEQ ID NO: 340 - DNA, 70 bp, Artificial Sequence
  Primer
gcagcacttgtaataaccaaattagcagcaggagacagatcaggtcttacagcagtaattaggagggcaa

SEQ ID NO: 341 - DNA, 70 bp, Artificial Sequence
  Primer
caagagggggtagtagagttgaaggaatctttgcaggattgtttatgaatgcctatggttcagggcaagt

SEQ ID NO: 342 - DNA, 70 bp, Artificial Sequence
  Primer
gacttaacagcagaagaattggaagccataaagaatcaactcaaccctaaagaagatgatgtagagcttt

SEQ ID NO: 343 - DNA, 70 bp, Artificial Sequence
  Primer
tcacaatccactgtgctcgacacaaccacattagaacacacaatccaacagcaatccctccactcaacca

SEQ ID NO: 344 - DNA, 70 bp, Artificial Sequence; identical to SEQ ID NO: 342
  Primer

SEQ ID NO: 345 - DNA, 70 bp, Artificial Sequence
  Primer
gccgacgaccatcaagcgtagccaaacaagatcagagagaacacagaattcagaactccacaaatcaaca

SEQ ID NO: 346 - DNA, 70 bp, Artificial Sequence
  Primer
cgacccaagatcatagatcaagtgaggagagtggaatctctaggagaacaggtgagtcaaaaactgagac

SEQ ID NO: 347 - DNA, 70 bp, Artificial Sequence
  Primer
cgcaaatgaagagggaaccagcaacacatcagtcgatgagatggccaagttactagtaagtcttggtgta

SEQ ID NO: 348 - DNA, 70 bp, Artificial Sequence
  Primer
ctccttgcaatggccatacgtagtccggaattatatctcactacaaacggtgtcaatgctgatgtcaagt

SEQ ID NO: 349 - DNA, 70 bp, Artificial Sequence
  Primer
gaacaaaaacagatgggttcattgtcaaaacgagagacatggagtatgaaagaaccacagagtggttgtt

SEQ ID NO: 350 - DNA, 70 bp, Artificial Sequence
  Primer
tgttccaagggcaaagagagaatgcggatctagaggcattgcttcagacatatggatatcctgcatgtct

SEQ ID NO: 351 - DNA, 70 bp, Artificial Sequence
  Primer
ggtatatccctcttcccagccacatcatgacaaaaggggcatttctaggtggagcagatatcaaagaatg

SEQ ID NO: 352 - DNA, 70 bp, Artificial Sequence
  Primer
gtataacaaccacatgtacatgcaacggtattggcaatagaatcaatcaaccacctgatcaaggagtaaa

SEQ ID NO: 353 - DNA, 70 bp, Artificial Sequence
  Primer
cccaacccattcaaaacgaaaatctcaaaagagattggcaacacaacaaacactgaacatcatgccaacc

SEQ ID NO: 354 - DNA, 70 bp, Artificial Sequence; identical to SEQ ID NO: 122
  Primer

SEQ ID NO: 355 - DNA, 70 bp, Artificial Sequence; identical to SEQ ID NO: 123
  Primer

SEQ ID NO: 356 - DNA, 70 bp, Artificial Sequence; identical to SEQ ID NO: 124
  Primer

SEQ ID NO: 357 - DNA, 70 bp, Artificial Sequence; identical to SEQ ID NO: 125
  Primer

SEQ ID NO: 358 - DNA, 70 bp, Artificial Sequence; identical to SEQ ID NO: 126
  Primer

SEQ ID NO: 359 - DNA, 70 bp, Artificial Sequence; identical to SEQ ID NO: 127
  Primer

SEQ ID NO: 360 - DNA, 70 bp, Artificial Sequence; identical to SEQ ID NO: 128
  Primer

SEQ ID NO: 361 - DNA, 70 bp, Artificial Sequence; identical to SEQ ID NO: 129
  Primer

SEQ ID NO: 362 - DNA, 70 bp, Artificial Sequence; identical to SEQ ID NO: 130
  Primer

SEQ ID NO: 363 - DNA, 70 bp, Artificial Sequence; identical to SEQ ID NO: 131
  Primer

SEQ ID NO: 364 - DNA, 70 bp, Artificial Sequence; identical to SEQ ID NO: 132
  Primer

SEQ ID NO: 365 - DNA, 70 bp, Artificial Sequence; identical to SEQ ID NO: 133
  Primer

SEQ ID NO: 366 - DNA, 70 bp, Artificial Sequence; identical to SEQ ID NO: 134
  Primer

SEQ ID NO: 367 - DNA, 70 bp, Artificial Sequence; identical to SEQ ID NO: 135
  Primer

SEQ ID NO: 368 - DNA, 70 bp, Artificial Sequence; identical to SEQ ID NO: 136
  Primer

SEQ ID NO: 369 - DNA, 70 bp, Artificial Sequence; identical to SEQ ID NO: 137
  Primer

SEQ ID NO: 370 - DNA, 70 bp, Artificial Sequence
  Primer
acccttatcgttagttgccagcacttagggtgggaactctaacgagactgcctgggttaaccaggaggaa

SEQ ID NO: 371 - DNA, 70 bp, Artificial Sequence
  Primer
ataagagaggttggctaatatccaattgatttgagcgtaccaggtaaagaagcaccggctaactccgtgc

SEQ ID NO: 372 - DNA, 70 bp, Artificial Sequence
  Primer
catgggatcttaagttttagttgaatacttctggaaagttgaacgatacagggtgatagtcccgtaaacg

SEQ ID NO: 373 - DNA, 70 bp, Artificial Sequence
  Primer
gggtgctagcgttaatcggatttattgggcgtaaagggcgtgtaggcggaaaggaaagttagatgttaaa

SEQ ID NO: 374 - DNA, 70 bp, Artificial Sequence
  Primer
gccagggagttaagttaaacggcgagattaagggatttacattccggagtcgaagcgaaagcgagtttta

SEQ ID NO: 375 - DNA, 70 bp, Artificial Sequence; identical to SEQ ID NO: 374
  Primer

SEQ ID NO: 376 - DNA, 70 bp, Artificial Sequence
  Primer
ggtgttgaacctgagaaaaatatttacaccaaacctgtggcctcagattattgggatggatatagtggac

SEQ ID NO: 377 - DNA, 70 bp, Artificial Sequence
  Primer
actgaggagcatgaaataatgaagttttcttggagaggagtgactgctgatactagggctttgagaagat

SEQ ID NO: 378 - DNA, 70 bp, Artificial Sequence
  Primer
catggcgtgactaagcccaaacaagtgattaaattggatgcagatccagtagagtcccagtcaactctag

SEQ ID NO: 379 - DNA, 70 bp, Artificial Sequence
  Primer
gtgcagtgatggacattacaggagtgcagtcaaccttgagatttcgtgttccttggatttctgatacacc

SEQ ID NO: 380 - DNA, 70 bp, Artificial Sequence
  Primer
ccaaaagagatttaatttggttggatgaaaatggtttgctgttaggagttcacccaagattggcccagag

SEQ ID NO: 381 - DNA, 70 bp, Artificial Sequence
  Primer
agagatgctttggatagggtaacagcggcggatattggtgagttgttaagacaaaaaccattcaacgccg

SEQ ID NO: 382 - DNA, 70 bp, Artificial Sequence
  Primer
gctggatgtgtctgcggcgttttatcatattcctcttcatcctgctgctatgcctcatcttcttattggt

SEQ ID NO: 383 - DNA, 70 bp, Artificial Sequence
  Primer
atatacatcctttccatagctgctaggttgtactgccaactagattcttcgcgggacgtcctttgtctac

SEQ ID NO: 384 - DNA, 70 bp, Artificial Sequence
  Primer
attctttcccgatcatcagttggaccctgcattcggagccaattcaaacaatccagattgggacttcaac

SEQ ID NO: 385 - DNA, 70 bp, Artificial Sequence
  Primer
ctcatgttgctgtacaaaacctacggatggaaattgcacctgtattcccatcccatcatcttgggctttc

SEQ ID NO: 386 - DNA, 70 bp, Artificial Sequence
  Primer
agagtctagactcgtggtggacttctctcaattttctagggggagcacccgtgtgtcttggccaaaattc

SEQ ID NO: 387 - DNA, 70 bp, Artificial Sequence
  Primer
ccttggatggctttggggcatggacattgacccttataaagaatttggagctactgtggagttactctca

SEQ ID NO: 388 - DNA, 70 bp, Artificial Sequence
  Primer
tgggagacagcaagacacactccagtcaattcctggctaggcaacataatcatgtttgcccccacactgt

SEQ ID NO: 389 - DNA, 66 bp, Artificial Sequence
  Primer
tgagcgactttaagacctggctgaaagccaagctcatgccacaactgcctgggattccctttgtgt

SEQ ID NO: 390 - DNA, 70 bp, Artificial Sequence
  Primer
tatagatgcccactttctatcccagacaaagcagagtggggagaactttccttacctggtagcgtaccaa

SEQ ID NO: 391 - DNA, 70 bp, Artificial Sequence
  Primer
taacaacaccaggccaccgctgggcaattggttcggttgtacctggatgaactcaactggattcaccaaa

SEQ ID NO: 392 - DNA, 70 bp, Artificial Sequence
  Primer
tttatccctgtggagaacctagagacaaccatgagatccccggtgttcacggacaactcctctccaccag

SEQ ID NO: 393 - DNA, 70 bp, Artificial Sequence; identical to SEQ ID NO: 392
  Primer

SEQ ID NO: 394 - DNA, 70 bp, Artificial Sequence
  Primer
ttcccttctctcgtcttcctcggtcaacctcttaagttcctcttcttcttccttgctgaggtgcttccct

SEQ ID NO: 395 - DNA, 70 bp, Artificial Sequence
  Primer
taagcccatagcgatagggagagatgctaggagttagaggagaccgaagcgaggaggaaagcaaagagag

SEQ ID NO: 396 - DNA, 70 bp, Artificial Sequence
  Primer
ttggagagcactccggccgaaaggtcgaggtacccagaaggaggaatctcacggagaaaagcagacaaat

SEQ ID NO: 397 - DNA, 70 bp, Artificial Sequence
  Primer
ttaagttcctcttcttcttccttgctgaggtgcttccctcccgcggccagctgctttctcttgttctcga

SEQ ID NO: 398 - DNA, 70 bp, Artificial Sequence
  Primer
aaaaagagaaagcaagagacggacgatttccccatgactctggagacatcctggaaggggaaagaaggaa

SEQ ID NO: 399 - DNA, 70 bp, Artificial Sequence
  Primer
aagttcctcttcttcttccttgctgaggtgcttccctcccgcggccagctgctttctcttgttctcgagg

SEQ ID NO: 400 - DNA, 66 bp, Artificial Sequence
  Primer
tcatatcatgcatcattggacacggcccccttctgctccacttggcttgctgagtgcaatgcagat

SEQ ID NO: 401 - DNA, 70 bp, Artificial Sequence
  Primer
taaagtgggaaagtgagttttggagatggactgaacagctggcctccaactactggattctggaatacct

SEQ ID NO: 402 - DNA, 70 bp, Artificial Sequence
  Primer
taggtcgtaaatcccggtcaccttggtagccactataggtgggtcttaagagaaggttaagattcctctt

SEQ ID NO: 403 - DNA, 66 bp, Artificial Sequence
  Primer
ttcttggtttgcctccaccagtggtcgcgactcgaagatagatgtgtggagtttagtgccagttgg

SEQ ID NO: 404 - DNA, 70 bp, Artificial Sequence
  Primer
tccaactactggattctggaatacctctggaaggtcccatttgatttctggagaggcgtgataagcctga

SEQ ID NO: 405 - DNA, 70 bp, Artificial Sequence
  Primer
acgttaccaaggtcttcatgtatcccggacagttactttcagcaagttgactattgcgacaaggtctcag

SEQ ID NO: 406 - DNA, 70 bp, Artificial Sequence
  Primer
tgtcagtaacaggggtcgccatagacttcggcctccattttaccttgtaaaaactaccaaaatggccgtt

SEQ ID NO: 407 - DNA, 70 bp, Artificial Sequence
  Primer
atgtcatccatttcctgggccgggtctacgtcctcatataagtaactgcacttccgaatggctgagtttt

SEQ ID NO: 408 - DNA, 70 bp, Artificial Sequence
  Primer
gggatctagcatccttatttcaaatagcaccataaacatgtttggtgaccccaaaccttacaacccttcc

SEQ ID NO: 409 - DNA, 70 bp, Artificial Sequence
  Primer
tgttagaaatccctgcaaagaaacccactcctcgggcaatagagtccctagaagcttacaaatcgttgac

SEQ ID NO: 410 - DNA, 70 bp, Artificial Sequence
  Primer
tcaaggattgacgtaaaggttaaaggtcatcctcggcggaagctacacaaaatggtggacaacatcttcc

SEQ ID NO: 411 - DNA, 70 bp, Artificial Sequence
  Primer
ggcatggttaactggaataatgaaaactttccatttaatgatgtagcagggaaaagcttggtggtctggg

SEQ ID NO: 412 - DNA, 70 bp, Artificial Sequence
  Primer
ggcaagaaaaatacactgtggttttatgggccgccaagtacaggaaaaacaaacttggcaatggccattg

SEQ ID NO: 413 - DNA, 70 bp, Artificial Sequence
  Primer
gccatttctcatggtcagaccacttatggtaacgctgaagacaaagagtatcagcaaggagtgggtagat

SEQ ID NO: 414 - DNA, 70 bp, Artificial Sequence
  Primer
aatttcgagaatttaccccagatttggtgcggtgtagctgccatgtgggagcttctaatcccttttctgt

SEQ ID NO: 415 - DNA, 70 bp, Artificial Sequence
  Primer
aggtgcgcaacgcttttatgaaggtaaagcccgtggcccaggagattatccgtatctgcatactcgctaa

SEQ ID NO: 416 - DNA, 70 bp, Artificial Sequence
  Primer
taaacgacatgtatctgttgttgacgctgcgacacttgcagctgcgacacgcgctggagctacaaatgat

SEQ ID NO: 417 - DNA, 70 bp, Artificial Sequence
  Primer
caaagcagcgtcaacaacagccacacagaaacctacgtggagacgacacgggactttttattgacggaga

SEQ ID NO: 418 - DNA, 70 bp, Artificial Sequence
  Primer
tgctccaaagcagcgtcaacaacagccacacagaaacctacgtggagacgacacgggactttttattgac

SEQ ID NO: 419 - DNA, 70 bp, Artificial Sequence
  Primer
gagttaaaagcaactactgtttattttccaaaatgagctgggtatagttgatgatctgtaggcgcagctc

SEQ ID NO: 420 - DNA, 70 bp, Artificial Sequence
  Primer
acagtgacagtgggagaaacacggcctctgagacatgtatgggggtgttcatctcacgcagaaaatcttt

SEQ ID NO: 421 - DNA, 70 bp, Artificial Sequence
  Primer
tgaagaagtcccgtagtgaaaaatgggatctgtctacaccatgtctggtgtgccgggaacatattgatcg

SEQ ID NO: 422 - DNA, 70 bp, Artificial Sequence; identical to SEQ ID NO: 421
  Primer

SEQ ID NO: 423 - DNA, 70 bp, Artificial Sequence
  Primer
attattgtctggtatagtgcagcagcagaacaatttgctgagggctattgaggcgcaacagcatctgttg

SEQ ID NO: 424 - DNA, 70 bp, Artificial Sequence
  Primer
gcaaccctctattgtgtgcatcaaaggatagagataaaagacaccaaggaagctttagacaagatagagg

SEQ ID NO: 425 - DNA, 70 bp, Artificial Sequence
  Primer
tgtatgtaggatctgacttagaaatagggcagcatagaacaaaaatagaggagctgagacaacatctgtt

SEQ ID NO: 426 - DNA, 70 bp, Artificial Sequence
  Primer
ggaatgctagttggagtaataaatctctggaacagatttggaatcacacgacctggatggagtgggacag

SEQ ID NO: 427 - DNA, 70 bp, Artificial Sequence
  Primer
taccttgaaagacgttaccgccaaaatgctcatcaaaagaacgaggaccatgctgacagcacccgcgaca

SEQ ID NO: 428 - DNA, 70 bp, Artificial Sequence
  Primer
tttcgtgatccttttccttttcctgtagctcagcgtcctttttatctaattcctctgcacgctccccgag

SEQ ID NO: 429 - DNA, 70 bp, Artificial Sequence
  Primer
tctttctgactcgcgcaaaaggcattactggaacactattttagccatgtggtggctccctgctatctta

SEQ ID NO: 430 - DNA, 70 bp, Artificial Sequence
  Primer
accttgaaagacgttaccgccaaaatgctcatcaaaagaacgaggaccatgctgacagcacccgcgacaa

SEQ ID NO: 431 - DNA, 70 bp, Artificial Sequence
  Primer
aataattcacgccgtcgctcctgattataggttggaacataacccaaagatgcttgaggctgcctaccgg

SEQ ID NO: 432 - DNA, 70 bp, Artificial Sequence
  Primer
tttgttgacggggcggttttagagactaatggcccagagcgccacaatctctcttttgatgccagtcaga

SEQ ID NO: 433 - DNA, 70 bp, Artificial Sequence
  Primer
attttactagtactaatggtgtcggtgagatcggccgcgggatagcgcttaccctgtttaaccttgctga

SEQ ID NO: 434 - DNA, 66 bp, Artificial Sequence
  Primer
agtccacttacggctcttcgaccggcccagtctatgtctctgactctgtgaccttggttaatgtag

SEQ ID NO: 435 - DNA, 70 bp, Artificial Sequence
  Primer
ggtatagtgtgggttgctgctaagggtgctgatactaaatctagatccaatcagggtacaagagatcctg

SEQ ID NO: 436 - DNA, 70 bp, Artificial Sequence; identical to SEQ ID NO: 435
  Primer

SEQ ID NO: 437 - DNA, 70 bp, Artificial Sequence
  Primer
ccagcccaagcaagtaacgaagcaaagtgccaaagaagtcaggcagaaaattttaaacaagcctcgccaa

SEQ ID NO: 438 - DNA, 70 bp, Artificial Sequence
  Primer
tctaaactttaaggatgtcttttgttcctgggcaagaaaatgccggtggcagaagctcctctgtaaaccg

SEQ ID NO: 439 - DNA, 70 bp, Artificial Sequence
  Primer
aggatcaagaaatagatccaattccggcactagaacacccacctctggtgtgacatctgatatggctgat

SEQ ID NO: 440 - DNA, 70 bp, Artificial Sequence
  Primer
tttaaaacagccgatggcaatcaacgccaattgttgccacgctggtatttttactacttgggaacaggcc

SEQ ID NO: 441 - DNA, 70 bp, Artificial Sequence
  Primer
ttggaacttatgtccgagagactttgtacccaaaggaataggtaacaaggatcaacagattggttattgg

SEQ ID NO: 442 - DNA, 70 bp, Artificial Sequence
  Primer
gctgaatgtgttccatctgtatctagcattctgtttggaagctattggactgcaaaggaagatggcgacc

SEQ ID NO: 443 - DNA, 70 bp, Artificial Sequence
  Primer
caccaccctcgaacaaggagctaaattttggtatgtatgtccgagagactttgttcccaagggaataggt

SEQ ID NO: 444 - DNA, 70 bp, Artificial Sequence
  Primer
ggcactcgtggaaccaacaatgaatccgaaccattgagatttgatggtaagataccaccacaattccagc

SEQ ID NO: 445 - DNA, 70 bp, Artificial Sequence
  Primer
ctgatccaaatgttgagcttcttgttgcacaggtggatgcatttaaaactgggaatgcaaaaccccagag

SEQ ID NO: 446 - DNA, 70 bp, Artificial Sequence
  Primer
atgagcaaattcgctggcgtatgcgccgtggtgagcgaattgaacaaccttcaaattggcatttctacta

SEQ ID NO: 447 - DNA, 70 bp, Artificial Sequence
  Primer
gagagactttgtacccaaaggaataggtaacagggatcaacagattggttattggaatagacaaactcgc

SEQ ID NO: 448 - DNA, 70 bp, Artificial Sequence
  Primer
gatggtgaccagatagaagtcacgttcacacacaaataccacttgccaaaggatgatcctaaaactggac

SEQ ID NO: 449 - DNA, 70 bp, Artificial Sequence
  Primer
tatttttactatcttggaacaggaccgcatgccaaagaccagtatggcaccgacattgacggagtctact

SEQ ID NO: 450 - DNA, 70 bp, Artificial Sequence
  Primer
agaacccctacctctggtgtaacacctgatatggctgatcaaattgctagtcttgttctggctaaacttg

SEQ ID NO: 451 - DNA, 70 bp, Artificial Sequence
  Primer
gagtgtggttaatcaacagggtgaagcgctgagtcaacttaccagtcagttacagaaaaacttccaggct

SEQ ID NO: 452 - DNA, 70 bp, Artificial Sequence
  Primer
ccggcattgtagatggtaataagatggccatgtacacagcatctttaattggaggtatggctttgggctc

SEQ ID NO: 453 - DNA, 70 bp, Artificial Sequence
  Primer
aaatgttaaaacttggaactagtgatccacagttccccattcttgcagagttggccccaacacctggtgc

SEQ ID NO: 454 - DNA, 70 bp, Artificial Sequence
  Primer
cccattactcttggttttcgggcattacccaatttcaaaagggaaaggagttccagtttgcagatgggca

SEQ ID NO: 455 - DNA, 70 bp, Artificial Sequence
  Primer
tagtaaccaggctgatattaataccccggctgacattgtcgatcgggatccaagtagcgatgaggctatt

SEQ ID NO: 456 - DNA, 70 bp, Artificial Sequence
  Primer
ttcttttaaaacagccgatggcaatcagcgtcaactgctgccacgatggtacttttactacctgggaaca

SEQ ID NO: 457 - DNA, 70 bp, Artificial Sequence
  Primer
gtggttccccattactcctggttttctggcattacccaattccagaagggaaaggagtttaagtttgcag

SEQ ID NO: 458 - DNA, 70 bp, Artificial Sequence
  Primer
aagaagtcaggcagaaaattttaaacaagcctcgccaaaagaggactccaaacaagcagtgcccagtgca

SEQ ID NO: 459 - DNA, 70 bp, Artificial Sequence
  Primer
tttggtgatgacaagatgaatgaggaaggtattaaggatgggcgtgttacggcaatgctcaacctagtcc

SEQ ID NO: 460 - DNA, 70 bp, Artificial Sequence; identical to SEQ ID NO: 459
  Primer

SEQ ID NO: 461 - DNA, 70 bp, Artificial Sequence
  Primer
agcctgcctctactgtaaaacctgatatggccgaagaaattgctgctcttgttttggctaagctaggcaa

SEQ ID NO: 462 - DNA, 70 bp, Artificial Sequence
  Primer
ccccattcttgcagagttggccccaacacctggtgccttcttctttggatctaaattagaattggtcaaa

SEQ ID NO: 463 - DNA, 26 bp, Artificial Sequence
  Primer
cacgtctcccaaatgcttgagtgacg

SEQ ID NO: 464 - DNA, 20 bp, Artificial Sequence
  Primer
cctcgaggccagggcgttcc

SEQ ID NO: 465 - DNA, 42 bp, Artificial Sequence
  Primer
tcacttgcttccgttgaggtcggggaccaagacctaatcaga

SEQ ID NO: 466 - DNA, 43 bp, Artificial Sequence
  Primer
ggtttcggatgttacagcgtagccgcaggaagaagagtcacag

SEQ ID NO: 467 - DNA, 38 bp, Artificial Sequence
  Primer
tcacttgcttccgttgaggaggccagggcgttccaatc

SEQ ID NO: 468 - DNA, 41 bp, Artificial Sequence
  Primer
ggtttcggatgttacagcgtcaatagcgcgagggcagtttc

SEQ ID NO: 469 - DNA, 42 bp, Artificial Sequence
  Primer
tcacttgcttccgttgaggggcacccgcaatcctaataacaa

SEQ ID NO: 470 - DNA, 43 bp, Artificial Sequence; identical to SEQ ID NO: 466
  Primer

SEQ ID NO: 471 - DNA, 23 bp, Artificial Sequence
  Primer
tcggggaccaagacctaatcaga

SEQ ID NO: 472 - DNA, 23 bp, Artificial Sequence
  Primer
agccgcaggaagaagagtcacag

SEQ ID NO: 473 - DNA, 19 bp, Artificial Sequence
  Primer
aggccagggcgttccaatc

SEQ ID NO: 474 - DNA, 21 bp, Artificial Sequence
  Primer
caatagcgcgagggcagtttc

SEQ ID NO: 475 - DNA, 23 bp, Artificial Sequence
  Primer
ggcacccgcaatcctaataacaa

SEQ ID NO: 476 - DNA, 23 bp, Artificial Sequence
  Primer
agccgcaggaagaagagtcacag

SEQ ID NO: 477 - DNA, 28 bp, Artificial Sequence
  Primer
acatcacagcttctacacccgttaaggt

SEQ ID NO: 478 - DNA, 28 bp, Artificial Sequence
  Primer
atacagaatacatagattgctgttatcc

SEQ ID NO: 479 - DNA, 27 bp, Artificial Sequence
  Primer
gcatcgttgactatggtgtccgattct

SEQ ID NO: 480 - DNA, 28 bp, Artificial Sequence
  Primer
gctgcattggtttgttatatcgttatgc

SEQ ID NO: 481 - DNA, 42 bp, Artificial Sequence
  Primer
tcacttgcttccgttgaggagccgcttgtcacaatgccaatt

SEQ ID NO: 482 - DNA, 43 bp, Artificial Sequence
  Primer
ggtttcggatgttacagcgtcatcaccaagctcgccaacagtt

SEQ ID NO: 483 - DNA, 43 bp, Artificial Sequence
  Primer
tcacttgcttccgttgaggaggttgccatcattttggcatctt

SEQ ID NO: 484 - DNA, 43 bp, Artificial Sequence
  Primer
ggtttcggatgttacagcgtctttgcgccagcgatagtgactt

SEQ ID NO: 485 - DNA, 41 bp, Artificial Sequence
  Primer
tcacttgcttccgttgaggatggcacccgtttctgcaatgg

SEQ ID NO: 486 - DNA, 44 bp, Artificial Sequence
  Primer
ggtttcggatgttacagcgttcgggcagctgacacgaatgtaga

SEQ ID NO: 487 - DNA, 43 bp, Artificial Sequence
  Primer
tcacttgcttccgttgagggaatggcgatgtagtggctattga

SEQ ID NO: 488 - DNA, 43 bp, Artificial Sequence
  Primer
ggtttcggatgttacagcgttaatgccggcatccaaacataat

SEQ ID NO: 489 - DNA, 43 bp, Artificial Sequence
  Primer
tcacttgcttccgttgaggtagccagcgtggtggttcatacaa

SEQ ID NO: 490 - DNA, 43 bp, Artificial Sequence
  Primer
ggtttcggatgttacagcgtctcccggcagaaagctgtaagct

SEQ ID NO: 491 - DNA, 42 bp, Artificial Sequence
  Primer
tcacttgcttccgttgaggtatagagcccgtgctggtgatgc

SEQ ID NO: 492 - DNA, 44 bp, Artificial Sequence
  Primer
ggtttcggatgttacagcgtatcgccattcaagtctgggaagaa

SEQ ID NO: 493 - DNA, 43 bp, Artificial Sequence
  Primer
tcacttgcttccgttgaggtggctcaggccatactggcattac

SEQ ID NO: 494 - DNA, 43 bp, Artificial Sequence
  Primer
ggtttcggatgttacagcgttttgcgccagcgatagtgacttg

SEQ ID NO: 495 - DNA, 42 bp, Artificial Sequence
  Primer
tcacttgcttccgttgaggttcccgtcaggcaaagttgaagg

SEQ ID NO: 496 - DNA, 44 bp, Artificial Sequence
  Primer
ggtttcggatgttacagcgtgacggcaattcctgtttgagcaga

SEQ ID NO: 497 - DNA, 23 bp, Artificial Sequence
  Primer
agccgcttgtcacaatgccaatt

SEQ ID NO: 498 - DNA, 23 bp, Artificial Sequence
  Primer
catcaccaagctcgccaacagtt

SEQ ID NO: 499 - DNA, 24 bp, Artificial Sequence
  Primer
aggttgccatcattttggcatctt

SEQ ID NO: 500 - DNA, 23 bp, Artificial Sequence
  Primer
ctttgcgccagcgatagtgactt

SEQ ID NO: 501 - DNA, 22 bp, Artificial Sequence
  Primer
atggcacccgtttctgcaatgg

SEQ ID NO: 502 - DNA, 24 bp, Artificial Sequence
  Primer
tcgggcagctgacacgaatgtaga

SEQ ID NO: 503 - DNA, 24 bp, Artificial Sequence
  Primer
gaatggcgatgtagtggctattga

SEQ ID NO: 504 - DNA, 23 bp, Artificial Sequence
  Primer
taatgccggcatccaaacataat

SEQ ID NO: 505 - DNA, 24 bp, Artificial Sequence
  Primer
tagccagcgtggtggttcatacaa

SEQ ID NO: 506 - DNA, 23 bp, Artificial Sequence
  Primer
ctcccggcagaaagctgtaagct

SEQ ID NO: 507 - DNA, 23 bp, Artificial Sequence
  Primer
tatagagcccgtgctggtgatgc

SEQ ID NO: 508 - DNA, 24 bp, Artificial Sequence
  Primer
atcgccattcaagtctgggaagaa

SEQ ID NO: 509 - DNA, 24 bp, Artificial Sequence
  Primer
tggctcaggccatactggcattac

SEQ ID NO: 510 - DNA, 23 bp, Artificial Sequence
  Primer
tttgcgccagcgatagtgacttg

SEQ ID NO: 511 - DNA, 23 bp, Artificial Sequence
  Primer
ttcccgtcaggcaaagttgaagg

SEQ ID NO: 512 - DNA, 24 bp, Artificial Sequence
  Primer
gacggcaattcctgtttgagcaga

SEQ ID NO: 513 - DNA, 43 bp, Artificial Sequence
  Primer
tcacttgcttccgttgaggatgaattaccaagtcaatggttac

SEQ ID NO: 514 - DNA, 41 bp, Artificial Sequence
  Primer
ggtttcggatgttacagcgtataaccagtcggtacagctac

SEQ ID NO: 515 - DNA, 39 bp, Artificial Sequence
  Primer
tcacttgcttccgttgagggaagctattcgtcacgttcg

SEQ ID NO: 516 - DNA, 42 bp, Artificial Sequence
  Primer
ggtttcggatgttacagcgtctgtagaaaatcctagctggag

SEQ ID NO: 517 - DNA, 40 bp, Artificial Sequence
  Primer
tcacttgcttccgttgaggcctctcttgttcttgctcgca

SEQ ID NO: 518 - DNA, 37 bp, Artificial Sequence
  Primer
ggtttcggatgttacagcgtgtgagccgccacacatg

SEQ ID NO: 519 - DNA, 40 bp, Artificial Sequence
  Primer
tcacttgcttccgttgaggctaacatgcttaggataatgg

SEQ ID NO: 520 - DNA, 41 bp, Artificial Sequence
  Primer
ggtttcggatgttacagcgtcaggtaagcgtaaaactcatc

SEQ ID NO: 521 - DNA, 40 bp, Artificial Sequence
  Primer
tcacttgcttccgttgagggcctctcttgttcttgctcgc

SEQ ID NO: 522 - DNA, 45 bp, Artificial Sequence
  Primer
tcacttgcttccgttgaggcaccgtttctacaggttagctaacga

SEQ ID NO: 523 - DNA, 46 bp, Artificial Sequence
  Primer
ggtttcggatgttacagcgtaaatgtttacgcaggtaagcgtaaaa

SEQ ID NO: 524 - DNA, 36 bp, Artificial Sequence
  Primer
tcacttgcttccgttgaggtacacacctcagcgttg

SEQ ID NO: 525 - DNA, 36 bp, Artificial Sequence
  Primer
ggtttcggatgttacagcgtcacgaacgtgacgaat

SEQ ID NO: 526 - DNA, 39 bp, Artificial Sequence
  Primer
tcacttgcttccgttgagggcttaggataatggcctctc

SEQ ID NO: 527 - DNA, 44 bp, Artificial Sequence
  Primer
ggtttcggatgttacagcgtccacgaattcatgatcaacatccc

SEQ ID NO: 528 - DNA, 40 bp, Artificial Sequence
  Primer
tcacttgcttccgttgagggctcgcaaacataacacttgc

SEQ ID NO: 529 - DNA, 41 bp, Artificial Sequence
  Primer
ggtttcggatgttacagcgtgagacactcatagagcctgtg

SEQ ID NO: 530 - DNA, 24 bp, Artificial Sequence
  Primer
atgaattaccaagtcaatggttac

SEQ ID NO: 531 - DNA, 21 bp, Artificial Sequence
  Primer
ataaccagtcggtacagctac

SEQ ID NO: 532 - DNA, 20 bp, Artificial Sequence
  Primer
gaagctattcgtcacgttcg

SEQ ID NO: 533 - DNA, 22 bp, Artificial Sequence
  Primer
ctgtagaaaatcctagctggag

SEQ ID NO: 534 - DNA, 21 bp, Artificial Sequence
  Primer
cctctcttgttcttgctcgca

SEQ ID NO: 535 - DNA, 17 bp, Artificial Sequence
  Primer
gtgagccgccacacatg

SEQ ID NO: 536 - DNA, 21 bp, Artificial Sequence
  Primer
ctaacatgcttaggataatgg

SEQ ID NO: 537 - DNA, 21 bp, Artificial Sequence
  Primer
caggtaagcgtaaaactcatc

SEQ ID NO: 538 - DNA, 21 bp, Artificial Sequence
  Primer
gcctctcttgttcttgctcgc

SEQ ID NO: 539 - DNA, 26 bp, Artificial Sequence
  Primer
caccgtttctacaggttagctaacga

SEQ ID NO: 540 - DNA, 26 bp, Artificial Sequence
  Primer
aaatgtttacgcaggtaagcgtaaaa

SEQ ID NO: 541 - DNA, 17 bp, Artificial Sequence
  Primer
tacacacctcagcgttg

SEQ ID NO: 542 - DNA, 16 bp, Artificial Sequence
  Primer
cacgaacgtgacgaat

SEQ ID NO: 543 - DNA, 20 bp, Artificial Sequence
  Primer
gcttaggataatggcctctc

SEQ ID NO: 544 - DNA, 24 bp, Artificial Sequence
  Primer
ccacgaattcatgatcaacatccc

SEQ ID NO: 545 - DNA, 21 bp, Artificial Sequence
  Primer
gctcgcaaacataacacttgc

SEQ ID NO: 546 - DNA, 21 bp, Artificial Sequence
  Primer
gagacactcatagagcctgtg

SEQ ID NO: 547 - DNA, 26 bp, Artificial Sequence
  Primer
ccagctccaataggaatgtcgcactc

SEQ ID NO: 548 - DNA, 28 bp, Artificial Sequence
  Primer
tccgcagatgtacatattacaatctacg

SEQ ID NO: 549 - DNA, 23 bp, Artificial Sequence
  Primer
ttaaatgcaccggccacggtttg

SEQ ID NO: 550 - DNA, 24 bp, Artificial Sequence
  Primer
atagcgccaggacaaactggtgtt

SEQ ID NO: 551 - DNA, 24 bp, Artificial Sequence
  Primer
tatatgcgccaagctggtgtgagt

SEQ ID NO: 552 - DNA, 24 bp, Artificial Sequence
  Primer
cgaggcggaggtacaaattgacag

SEQ ID NO: 553 - DNA, 24 bp, Artificial Sequence
  Primer
atgaagccgagccaaacataccaa

SEQ ID NO: 554 - DNA, 40 bp, Artificial Sequence
  Primer
tcacttgcttccgttgaggatgcaccggccacggtttgtg

SEQ ID NO: 555 - DNA, 44 bp, Artificial Sequence
  Primer
ggtttcggatgttacagcgtatgcgccaagctggtgtgagttga

SEQ ID NO: 556 - DNA, 43 bp, Artificial Sequence
  Primer
tcacttgcttccgttgaggtgctggcgctgctcttcaaatacc

SEQ ID NO: 557 - DNA, 40 bp, Artificial Sequence
  Primer
ggtttcggatgttacagcgtcggggctgcttgtgggaagg

SEQ ID NO: 558 - DNA, 43 bp, Artificial Sequence
  Primer
tcacttgcttccgttgaggatagcgccaggacaaactggtgtt

SEQ ID NO: 559 - DNA, 44 bp, Artificial Sequence
  Primer
ggtttcggatgttacagcgttatatgcgccaagctggtgtgagt

SEQ ID NO: 560 - DNA, 43 bp, Artificial Sequence
  Primer
tcacttgcttccgttgaggcgaggcggaggtacaaattgacag

SEQ ID NO: 561 - DNA, 44 bp, Artificial Sequence
  Primer
ggtttcggatgttacagcgtatgaagccgagccaaacataccaa

SEQ ID NO: 562 - DNA, 21 bp, Artificial Sequence
  Primer
atgcaccggccacggtttgtg

SEQ ID NO: 563 - DNA, 24 bp, Artificial Sequence
  Primer
atgcgccaagctggtgtgagttga

SEQ ID NO: 564 - DNA, 24 bp, Artificial Sequence
  Primer
tgctggcgctgctcttcaaatacc

SEQ ID NO: 565 - DNA, 20 bp, Artificial Sequence
  Primer
cggggctgcttgtgggaagg

SEQ ID NO: 566 - DNA, 19 bp, Artificial Sequence
  Primer
tttgtgcgacaatgcttca

SEQ ID NO: 567 - DNA, 20 bp, Artificial Sequence
  Primer
gacatttgagaaagcttgcc

SEQ ID NO: 568 - DNA, 20 bp, Artificial Sequence
  Primer
  misc_feature 12: n = a, c, g, or t
agggacaacctngaacctgg

SEQ ID NO: 569 - DNA, 22 bp, Artificial Sequence
  Primer
aggagttgaaccaagacgcatt

SEQ ID NO: 570 - DNA, 22 bp, Artificial Sequence
  Primer
accacattcccttatactggag

SEQ ID NO: 571 - DNA, 24 bp, Artificial Sequence
  Primer
ttagtcatcatctttctcacaaca

SEQ ID NO: 572 - DNA, 22 bp, Artificial Sequence
  Primer
acaaattgcttcaaatgagaac

SEQ ID NO: 573 - DNA, 22 bp, Artificial Sequence
  Primer
tgtctccgaagaaataagatcc

SEQ ID NO: 574 - DNA, 20 bp, Artificial Sequence
  Primer
gcgcagagacttgaagatgt

SEQ ID NO: 575 - DNA, 18 bp, Artificial Sequence
  Primer
ccttccgtagaaggccct

SEQ ID NO: 576 - DNA, 21 bp, Artificial Sequence
  Primer
cacaatggcagaatttagtga

SEQ ID NO: 577 - DNA, 20 bp, Artificial Sequence
  Primer
gtcagtttgatcccgtagtg

SEQ ID NO: 578 - DNA, 20 bp, Artificial Sequence
  Primer
cagatcccagagtggactca

SEQ ID NO: 579 - DNA, 22 bp, Artificial Sequence
  Primer
tgtattacccaagggttgttac

SEQ ID NO: 580 - DNA, 22 bp, Artificial Sequence
  Primer
gatcagcatgacagtaacagga

SEQ ID NO: 581 - DNA, 22 bp, Artificial Sequence
  Primer
atgttcggtaaaagtcgtttat

SEQ ID NO: 582 - DNA, 20 bp, Artificial Sequence
  Primer
ccacaggggagattccaaag

SEQ ID NO: 583 - DNA, 23 bp, Artificial Sequence
  Primer
gacattcttcctgattcataatc

SEQ ID NO: 584 - DNA, 22 bp, Artificial Sequence
  Primer
caaacaacggtagaccaatata

SEQ ID NO: 585 - DNA, 24 bp, Artificial Sequence
  Primer
aggttcagtatctatcacagtctt

SEQ ID NO: 586 - DNA, 21 bp, Artificial Sequence
  Primer
atgtccaacatggatattgac

SEQ ID NO: 587 - DNA, 21 bp, Artificial Sequence
  Primer
gctcttcctataaatcgaatg

SEQ ID NO: 588 - DNA, 21 bp, Artificial Sequence
  Primer
tgatcaagtgatcggaagtag

SEQ ID NO: 589 - DNA, 20 bp, Artificial Sequence
  Primer
gatggtctgcttaattggaa

SEQ ID NO: 590 - DNA, 20 bp, Artificial Sequence
  Primer
acagaagatggagaaggcaa

SEQ ID NO: 591 - DNA, 20 bp, Artificial Sequence
  Primer
attgtttctttggcctggat

SEQ ID NO: 592 - DNA, 20 bp, Artificial Sequence
  Primer
tggcggtataggggtaactg

SEQ ID NO: 593 - DNA, 20 bp, Artificial Sequence
  Primer
attgcggtgatggttaaagg

SEQ ID NO: 594 - DNA, 20 bp, Artificial Sequence
  Primer
ttttgccgatcccacttatc

SEQ ID NO: 595 - DNA, 20 bp, Artificial Sequence
  Primer
gcaagtctaccacggcattt

SEQ ID NO: 596 - DNA, 20 bp, Artificial Sequence
  Primer
ctccgttatcgctccatgtt

SEQ ID NO: 597 - DNA, 20 bp, Artificial Sequence
  Primer
aaggactggtcgttggtgtc

SEQ ID NO: 598 - DNA, 20 bp, Artificial Sequence
  Primer
aaatgccgtggtagatttgc

SEQ ID NO: 599 - DNA, 20 bp, Artificial Sequence
  Primer
gttgaaggggttgacgttgt

SEQ ID NO: 600 - DNA, 20 bp, Artificial Sequence
  Primer
tcctctggatggcataggac

SEQ ID NO: 601 - DNA, 20 bp, Artificial Sequence
  Primer
tgttggtgttagtgggcaaa

SEQ ID NO: 602 - DNA, 20 bp, Artificial Sequence
  Primer
acatggtcctgcaaagttcc

SEQ ID NO: 603 - DNA, 20 bp, Artificial Sequence
  Primer
gcattgtgccacgttgtatc

SEQ ID NO: 604 - DNA, 20 bp, Artificial Sequence
  Primer
cgcttcggagtacctcagtc

SEQ ID NO: 605 - DNA, 20 bp, Artificial Sequence
  Primer
ctgcatcattggtgtcaacc

SEQ ID NO: 606 - DNA, 20 bp, Artificial Sequence
  Primer
ggcaccttttacctcaacca

SEQ ID NO: 607 - DNA, 20 bp, Artificial Sequence
  Primer
tctggaccaagaaccagtcc

SEQ ID NO: 608 - DNA, 20 bp, Artificial Sequence
  Primer
ggcctaccctgctaacttcc

SEQ ID NO: 609 - DNA, 20 bp, Artificial Sequence
  Primer
ataaagaagggtgggctcgt

SEQ ID NO: 610 - DNA, 20 bp, Artificial Sequence
  Primer
atcgcagttgaatgctgttg

SEQ ID NO: 611 - DNA, 20 bp, Artificial Sequence
  Primer
gttgaaggggttgacgttgt

SEQ ID NO: 612 - DNA, 20 bp, Artificial Sequence
  Primer
acatggtcctgcaaagttcc

SEQ ID NO: 613 - DNA, 20 bp, Artificial Sequence
  Primer
gatcgaaccctgatccaaga

SEQ ID NO: 614 - DNA, 20 bp, Artificial Sequence
  Primer
aacaccaaccgaaggagatg

SEQ ID NO: 615 - DNA, 20 bp, Artificial Sequence
  Primer
cctatgccatccagaggaaa

SEQ ID NO: 616 - DNA, 20 bp, Artificial Sequence
  Primer
cagatgctcgccaactacaa

SEQ ID NO: 617 - DNA, 20 bp, Artificial Sequence
  Primer
agccatgtaacccacaaagc

SEQ ID NO: 618 - DNA, 20 bp, Artificial Sequence
  Primer
acggacgttatgtgcctttc

SEQ ID NO: 619 - DNA, 20 bp, Artificial Sequence
  Primer
gggaatattggttgcattgg

SEQ ID NO: 620 - DNA, 20 bp, Artificial Sequence
  Primer
actggttcctggtccagatg

SEQ ID NO: 621 - DNA, 20 bp, Artificial Sequence
  Primer
agccatgtaacccacaaagc

SEQ ID NO: 622 - DNA, 20 bp, Artificial Sequence
  Primer
ctggatatggccagcacttt

SEQ ID NO: 623 - DNA, 20 bp, Artificial Sequence
  Primer
cacctgaggttctggttggt

SEQ ID NO: 624 - DNA, 20 bp, Artificial Sequence
  Primer
taatgaaaagggcggacaag

SEQ ID NO: 625 - DNA, 20 bp, Artificial Sequence
  Primer
gccaatgtagtttggcctgt

SEQ ID NO: 626 - DNA, 20 bp, Artificial Sequence
  Primer
aactccgcggtagacagcta

SEQ ID NO: 627 - DNA, 20 bp, Artificial Sequence
  Primer
cgtaggtgttggtgttggtg

SEQ ID NO: 628 - DNA, 43 bp, Artificial Sequence; identical to SEQ ID NO: 95
  Primer

SEQ ID NO: 629 - DNA, 43 bp, Artificial Sequence; identical to SEQ ID NO: 96
  Primer

SEQ ID NO: 630 - DNA, 24 bp, Artificial Sequence
  Primer
ttggggtgatgggtttcagattaa

SEQ ID NO: 631 - DNA, 23 bp, Artificial Sequence
  Primer
ctcgggaagatcgccttcttcta

SEQ ID NO: 632 - DNA, 42 bp, Artificial Sequence; identical to SEQ ID NO: 79
  Primer

SEQ ID NO: 633 - DNA, 42 bp, Artificial Sequence; identical to SEQ ID NO: 80
  Primer

SEQ ID NO: 634 - DNA, 43 bp, Artificial Sequence; identical to SEQ ID NO: 81
  Primer

SEQ ID NO: 635 - DNA, 44 bp, Artificial Sequence; identical to SEQ ID NO: 82
  Primer

SEQ ID NO: 636 - DNA, 41 bp, Artificial Sequence; identical to SEQ ID NO: 83
  Primer

SEQ ID NO: 637 - DNA, 44 bp, Artificial Sequence; identical to SEQ ID NO: 84
  Primer

SEQ ID NO: 638 - DNA, 43 bp, Artificial Sequence; identical to SEQ ID NO: 85
  Primer

SEQ ID NO: 639 - DNA, 44 bp, Artificial Sequence; identical to SEQ ID NO: 82
  Primer

SEQ ID NO: 640 - DNA, 23 bp, Artificial Sequence
  Primer
ttgggctggcggtttagagttga

SEQ ID NO: 641 - DNA, 22 bp, Artificial Sequence
  Primer
gtgcgaccgcccttgtttatgg

SEQ ID NO: 642 - DNA, 24 bp, Artificial Sequence
  Primer
gcgttgttggcctttttcttgtct

SEQ ID NO: 643 - DNA, 24 bp, Artificial Sequence
  Primer
gcccggcattatttcattgttctg

SEQ ID NO: 644 - DNA, 22 bp, Artificial Sequence
  Primer
acaaaagccgctggtggtaaag

SEQ ID NO: 645 - DNA, 24 bp, Artificial Sequence
  Primer
cagaaatcataacgggcaaactca

SEQ ID NO: 646 - DNA, 24 bp, Artificial Sequence
  Primer
aagagttattgctggcgttgttgg

SEQ ID NO: 647 - DNA, 24 bp, Artificial Sequence
  Primer
gcccggcattatttcattgttctg

SEQ ID NO: 648 - DNA, 20 bp, Artificial Sequence
  Primer
ggtggtaacccctcgcagga

SEQ ID NO: 649 - DNA, 21 bp, Artificial Sequence
  Primer
tggctcttccctttgggcact

SEQ ID NO: 650 - DNA, 26 bp, Artificial Sequence
  Primer
gagaatgaaccttatgtcggcacctg

SEQ ID NO: 651 - DNA, 26 bp, Artificial Sequence
  Primer
ttccgcaagtctttcactttctccaa

SEQ ID NO: 652 - DNA, 22 bp, Artificial Sequence
  Primer
cagctttcagccagggacgtgt

SEQ ID NO: 653 - DNA, 22 bp, Artificial Sequence
  Primer
tttccagcttttgcgcagtggt

SEQ ID NO: 654 - DNA, 26 bp, Artificial Sequence
  Primer
tctgttttggtgcaggtcaatttgtg

SEQ ID NO: 655 - DNA, 26 bp, Artificial Sequence
  Primer
atgaaccaggtcgtaagcatcctcaa

SEQ ID NO: 656 - DNA, 26 bp, Artificial Sequence
  Primer
gttgcttgtcaacccccgtactgtta

SEQ ID NO: 657 - DNA, 26 bp, Artificial Sequence
  Primer
aggacacctgccataggggtagagag

SEQ ID NO: 658 - DNA, 20 bp, Artificial Sequence
  Primer
ggttgttgactcgcggtgga

SEQ ID NO: 659 - DNA, 23 bp, Artificial Sequence
  Primer
ggggtagagaggccaaacactgc

SEQ ID NO: 660 - DNA, 20 bp, Artificial Sequence
  Primer
acatggtcccattggattgt

SEQ ID NO: 661 - DNA, 20 bp, Artificial Sequence
  Primer
tgaggaaatctttcgccact

SEQ ID NO: 662 - DNA, 20 bp, Artificial Sequence
  Primer
atgttgccccctagtctgtg

SEQ ID NO: 663 - DNA, 20 bp, Artificial Sequence
  Primer
ttctgaaggtggtgtgttgc

SEQ ID NO: 664 - DNA, 20 bp, Artificial Sequence
  Primer
tggtattcatgttggcggta

SEQ ID NO: 665 - DNA, 20 bp, Artificial Sequence
  Primer
acagcaggttccttgtcacc

SEQ ID NO: 666 - DNA, 20 bp, Artificial Sequence
  Primer
tcttgcctccaatggctagt

SEQ ID NO: 667 - DNA, 20 bp, Artificial Sequence
  Primer
tgacatgcctgcattgagtt

SEQ ID NO: 668 - DNA, 20 bp, Artificial Sequence
  Primer
tcccaatatgccctcttcag

SEQ ID NO: 669 - DNA, 20 bp, Artificial Sequence
  Primer
cgctgatggggattgagtat

SEQ ID NO: 670 - DNA, 20 bp, Artificial Sequence
  Primer
tgtgctcagtgtgcttcctc

SEQ ID NO: 671 - DNA, 20 bp, Artificial Sequence
  Primer
tgcacccatgatgacaatct

SEQ ID NO: 672 - DNA, 20 bp, Artificial Sequence
  Primer
gcagttcttgccaaagaagg

SEQ ID NO: 673 - DNA, 20 bp, Artificial Sequence
  Primer
tgaagggtttttggtccatc

SEQ ID NO: 674 - DNA, 20 bp, Artificial Sequence
  Primer
tgcctgatgcccttaaaaac

SEQ ID NO: 675 - DNA, 20 bp, Artificial Sequence
  Primer
gggtgtgattgtacccgact

SEQ ID NO: 676 - DNA, 26 bp, Artificial Sequence
  Primer
cttaacagttgtatgcattggaaact

SEQ ID NO: 677 - DNA, 26 bp, Artificial Sequence
  Primer
gtttacggtgtggactactagggtat

SEQ ID NO: 678 - DNA, 26 bp, Artificial Sequence
  Primer
ctatgctgagaagtagaatagccaca

SEQ ID NO: 679 - DNA, 26 bp, Artificial Sequence
  Primer
tggtacagtcaaactctagccattac

SEQ ID NO: 680 - DNA, 26 bp, Artificial Sequence
  Primer
ataccctagtagtccacaccgtaaac

SEQ ID NO: 681 - DNA, 26 bp, Artificial Sequence
  Primer
atgtcaagtctaggtaaggtttttcg

SEQ ID NO: 682 - DNA, 20 bp, Artificial Sequence
  Primer
aggcgaaaacttaggccatt

SEQ ID NO: 683 - DNA, 20 bp, Artificial Sequence
  Primer
ccgtcaattccgtttgagtt

SEQ ID NO: 684 - DNA, 20 bp, Artificial Sequence
  Primer
cgacggtacacgaaaaacct

SEQ ID NO: 685 - DNA, 20 bp, Artificial Sequence
  Primer
tcccttccttcctccaattt

SEQ ID NO: 686 - DNA, 24 bp, Artificial Sequence
  Primer
attcccatggagaaactcctagat

SEQ ID NO: 687 - DNA, 21 bp, Artificial Sequence
  Primer
gtgatcactgacctgcatctg

SEQ ID NO: 688 - DNA, 23 bp, Artificial Sequence
  Primer
gtaagagaccacgtccgatcaat

SEQ ID NO: 689 - DNA, 23 bp, Artificial Sequence
  Primer
gaggacgtgtagggcttctttag

SEQ ID NO: 690 - DNA, 20 bp, Artificial Sequence
  Primer
atcggacctcgcttaggact

SEQ ID NO: 691 - DNA, 20 bp, Artificial Sequence
  Primer
ctgggtatcacggctacgat

SEQ ID NO: 692 - DNA, 20 bp, Artificial Sequence
  Primer
agagaccacgtccgatcaat

SEQ ID NO: 693 - DNA, 20 bp, Artificial Sequence
  Primer
tgaggacgtgtagggcttct

SEQ ID NO: 694 - DNA, 20 bp, Artificial Sequence
  Primer
gtcaacgcctactcctctgg

SEQ ID NO: 695 - DNA, 20 bp, Artificial Sequence
  Primer
gtcttgtgagggtgctggac

SEQ ID NO: 696 - DNA, 20 bp, Artificial Sequence
  Primer
cacattggcatctgaactcg

SEQ ID NO: 697 - DNA, 20 bp, Artificial Sequence
  Primer
tctgtttgaccctcctgtcc

SEQ ID NO: 698 - DNA, 26 bp, Artificial Sequence
  Primer
agattgcaatgcatactactgaggac

SEQ ID NO: 699 - DNA, 25 bp, Artificial Sequence
  Primer
atgcagtgtcaatgtctagaggtgt

SEQ ID NO: 700 - DNA, 24 bp, Artificial Sequence
  Primer
caatgcatactactgaggacagga

SEQ ID NO: 701 - DNA, 24 bp, Artificial Sequence
  Primer
atgcagtgtcaatgtctagaggtg

SEQ ID NO: 702 - DNA, 24 bp, Artificial Sequence
  Primer
taccatcagaggtcaattctcaaa

SEQ ID NO: 703 - DNA, 24 bp, Artificial Sequence
  Primer
ctacttcaaacactcggtacatgc

SEQ ID NO: 704 - DNA, 24 bp, Artificial Sequence
  Primer
catgtcgctgtctctgttagactt

SEQ ID NO: 705 - DNA, 24 bp, Artificial Sequence
  Primer
caagcctggatttcttataacacc

SEQ ID NO: 706 - DNA, 23 bp, Artificial Sequence
  Primer
aaaccaaagaagaaaccaaccat

SEQ ID NO: 707 - DNA, 23 bp, Artificial Sequence
  Primer
tgttctaatgtggttgtgtcgag

SEQ ID NO: 708 - DNA, 23 bp, Artificial Sequence
  Primer
tgctaaaagagatgggagaagtg

SEQ ID NO: 709 - DNA, 23 bp, Artificial Sequence
  Primer
atcctttggtatgagacccttgt

SEQ ID NO: 710 - DNA, 23 bp, Artificial Sequence
  Primer
acaagggtctcataccaaaggat

SEQ ID NO: 711 - DNA, 23 bp, Artificial Sequence
  Primer
gctaaaactccccatcttagcat

SEQ ID NO: 712 - DNA, 20 bp, Artificial Sequence
  Primer
tttatgatgcagccaaagca

SEQ ID NO: 713 - DNA, 20 bp, Artificial Sequence
  Primer
tccatgaaattcaggtgcaa

SEQ ID NO: 714 - DNA, 20 bp, Artificial Sequence
  Primer
aaaaacaccagccaaaacga

SEQ ID NO: 715 - DNA, 20 bp, Artificial Sequence
  Primer
ctgtgggtgtttgtgtggag

SEQ ID NO: 716 - DNA, 20 bp, Artificial Sequence
  Primer
ccaaagcatatgcagagcaa

SEQ ID NO: 717 - DNA, 20 bp, Artificial Sequence
  Primer
tccatgaaattcaggtgcaa

SEQ ID NO: 718 - DNA, 20 bp, Artificial Sequence
  Primer
gcatggaaactagcagcaca

SEQ ID NO: 719 - DNA, 20 bp, Artificial Sequence
  Primer
ggtgttgtggtcttcgaggt

SEQ ID NO: 720 - DNA, 23 bp, Artificial Sequence
  Primer
ggctccatagtatcatcgacaac

SEQ ID NO: 721 - DNA, 23 bp, Artificial Sequence
  Primer
cctagaggccctgtgtatacctt

SEQ ID NO: 722 - DNA, 23 bp, Artificial Sequence
  Primer
acacaacaaacaatgcaaacaac

SEQ ID NO: 723 - DNA, 23 bp, Artificial Sequence
  Primer
ttaacatgcgcttagcaaataca

SEQ ID NO: 724 - DNA, 23 bp, Artificial Sequence
  Primer
ttagctcactcattggacacaga

SEQ ID NO: 725 - DNA, 23 bp, Artificial Sequence
  Primer
gtctctcgttttgacaatgaacc

SEQ ID NO: 726 - DNA, 23 bp, Artificial Sequence
  Primer
tctcactacaaacggtgtcaatg

SEQ ID NO: 727 - DNA, 23 bp, Artificial Sequence
  Primer
tctagatccgcattctctctttg

SEQ ID NO: 728 - DNA, 23 bp, Artificial Sequence
  Primer
acagatgggttcattgtcaaaac

SEQ ID NO: 729 - DNA, 23 bp, Artificial Sequence
  Primer
gctttgaccaacactatccaaac

SEQ ID NO: 730 - DNA, 23 bp, Artificial Sequence
  Primer
gctgaacacccagatttacaaag

SEQ ID NO: 731 - DNA, 23 bp, Artificial Sequence
  Primer
acagctctccatttcatggttta

SEQ ID NO: 732 - DNA, 23 bp, Artificial Sequence
  Primer
atatgcatttgtcaatggaggag

SEQ ID NO: 733 - DNA, 23 bp, Artificial Sequence
  Primer
catttggtgtgtaaaatgcaaga

SEQ ID NO: 734 - DNA, 23 bp, Artificial Sequence
  Primer
cacagaacaccagaacaacaaga

SEQ ID NO: 735 - DNA, 23 bp, Artificial Sequence
  Primer
ttgggactgttaaccaatacacc

SEQ ID NO: 736 - DNA, 20 bp, Artificial Sequence
  Primer
catcccaaaaattgccagat

SEQ ID NO: 737 - DNA, 20 bp, Artificial Sequence
  Primer
tttgggctttgccttaaatg

SEQ ID NO: 738 - DNA, 20 bp, Artificial Sequence
  Primer
acaccctcatcattgcaaca

SEQ ID NO: 739 - DNA, 20 bp, Artificial Sequence
  Primer
gcccttctgactgtggtctc

SEQ ID NO: 740 - DNA, 20 bp, Artificial Sequence
  Primer
cgacacagcagcaggaatta

SEQ ID NO: 741 - DNA, 20 bp, Artificial Sequence
  Primer
tcaaagctgcttgacactgg

SEQ ID NO: 742 - DNA, 20 bp, Artificial Sequence
  Primer
tggggccagacttcgccata

SEQ ID NO: 743 - DNA, 20 bp, Artificial Sequence
  Primer
agcttccgccgaggatgacc

SEQ ID NO: 744 - DNA, 20 bp, Artificial Sequence
  Primer
cttgggggctcaacgccttc

SEQ ID NO: 745 - DNA, 20 bp, Artificial Sequence
  Primer
gcgaagtctggccccactca

SEQ ID NO: 746 - DNA, 20 bp, Artificial Sequence
  Primer
ccacaggccaaccgaatgct

SEQ ID NO: 747 - DNA, 20 bp, Artificial Sequence
  Primer
agcccgaattgccccttgac

SEQ ID NO: 748 - DNA, 25 bp, Artificial Sequence
  Primer
agcgaatcctgggagtcaaactcag

SEQ ID NO: 749 - DNA, 25 bp, Artificial Sequence
  Primer
ggcctcgtactcctctttccagtca

SEQ ID NO: 750 - DNA, 25 bp, Artificial Sequence
  Primer
gcccctttgcataccactcagacat

SEQ ID NO: 751 - DNA, 25 bp, Artificial Sequence
  Primer
tggaatgtgagttccggtgagttgt

SEQ ID NO: 752 - DNA, 25 bp, Artificial Sequence
  Primer
tgtcagtaacaggggtcgccataga

SEQ ID NO: 753 - DNA, 25 bp, Artificial Sequence
  Primer
tgtgacgtatggacgacctttgacc

SEQ ID NO: 754 - DNA, 22 bp, Artificial Sequence
  Primer
cacagacagaggagaaggcaac

SEQ ID NO: 755 - DNA, 26 bp, Artificial Sequence
  Primer
aataggcacattactactacctcctg

SEQ ID NO: 756 - DNA, 25 bp, Artificial Sequence
  Primer
gcggtcggtaggaggataaaggaaa

SEQ ID NO: 757 - DNA, 25 bp, Artificial Sequence
  Primer
ccggggatttgtctacagggtttct

SEQ ID NO: 758 - DNA, 25 bp, Artificial Sequence
  Primer
cagacgctcatccaactcctgagaa

SEQ ID NO: 759 - DNA, 25 bp, Artificial Sequence
  Primer
ccgttgtaccgtctttttggacgtt

SEQ ID NO: 760 - DNA, 25 bp, Artificial Sequence
  Primer
cacgctctacctcattcgagagcaa

SEQ ID NO: 761 - DNA, 25 bp, Artificial Sequence
  Primer
gttgtgttgcaacgaacacgctaca

SEQ ID NO: 762 - DNA, 25 bp, Artificial Sequence
  Primer
agcggtcggtaggaggataaaggaa

SEQ ID NO: 763 - DNA, 25 bp, Artificial Sequence
  Primer
accggggatttgtctacagggtttc

SEQ ID NO: 764 - DNA, 24 bp, Artificial Sequence
  Primer
aacacgatccgctacgactactac

SEQ ID NO: 765 - DNA, 24 bp, Artificial Sequence
  Primer
ccctatacccgttcgcaatcaaag

SEQ ID NO: 766 - DNA, 20 bp, Artificial Sequence
  Primer
atgggcgcagcctcaatgac

SEQ ID NO: 767 - DNA, 20 bp, Artificial Sequence
  Primer
ccccaaatccccaggagctg

SEQ ID NO: 768 - DNA, 25 bp, Artificial Sequence
  Primer
gggacagctacaaccatcccttcag

SEQ ID NO: 769 - DNA, 25 bp, Artificial Sequence
  Primer
gacctgattgctgtgtcctgtgtca

SEQ ID NO: 770 - DNA, 25 bp, Artificial Sequence
  Primer
gggatggaaaggatcaccagcaata

SEQ ID NO: 771 - DNA, 25 bp, Artificial Sequence
  Primer
gtctggtgtggtaagtccccacctc

SEQ ID NO: 772 - DNA, 25 bp, Artificial Sequence
  Primer
aaggatcaacagctcctggggattt

SEQ ID NO: 773 - DNA, 25 bp, Artificial Sequence
  Primer
ttcttgctggttttgcgattcttca

SEQ ID NO: 774 - DNA, 27 bp, Artificial Sequence
  Primer
taatccacctatcccagtaggagaaat

SEQ ID NO: 775 - DNA, 25 bp, Artificial Sequence
  Primer
ggtccttgtcttatgtccagaatgc

SEQ ID NO: 776 - DNA, 25 bp, Artificial Sequence
  Primer
tgggaagttcaattaggaataccac

SEQ ID NO: 777 - DNA, 27 bp, Artificial Sequence
  Primer
tcctacatacaaatcatccatgtattg

SEQ ID NO: 778 - DNA, 20 bp, Artificial Sequence
  Primer
gccggcgatgactgcttgat

SEQ ID NO: 779 - DNA, 20 bp, Artificial Sequence
  Primer
tccggaagtccgtggtcagg

SEQ ID NO: 780 - DNA, 20 bp, Artificial Sequence
  Primer
acggtgggagtcgcgttgac

SEQ ID NO: 781 - DNA, 20 bp, Artificial Sequence
  Primer
ggccacgcaaaccaacaagg

SEQ ID NO: 782 - DNA, 20 bp, Artificial Sequence
  Primer
cggccaaaaggtggtggatg

SEQ ID NO: 783 - DNA, 20 bp, Artificial Sequence
  Primer
cgggctcggtttaacgacga

SEQ ID NO: 784 - DNA, 20 bp, Artificial Sequence
  Primer
gccacgggcaaaatcagtgg

SEQ ID NO: 785 - DNA, 20 bp, Artificial Sequence
  Primer
tgtcgcgatccgatgatcca

SEQ ID NO: 786 - DNA, 25 bp, Artificial Sequence
  Primer
cgcgtgtgagctaaagtgggaaagt

SEQ ID NO: 787 - DNA, 25 bp, Artificial Sequence
  Primer
atcgtcaccaacaggaagaccatga

SEQ ID NO: 788 - DNA, 25 bp, Artificial Sequence
  Primer
tcgctctcgggttggttttgtattc

SEQ ID NO: 789 - DNA, 25 bp, Artificial Sequence
  Primer
catccaccttaggctccctgttgac

SEQ ID NO: 790 - DNA, 22 bp, Artificial Sequence
  Primer
gggttggtaggtcgtaaatccc

SEQ ID NO: 791 - DNA, 19 bp, Artificial Sequence
  Primer
gtacgtgggcgtcgtttgc

SEQ ID NO: 792 - DNA, 20 bp, Artificial Sequence
  Primer
cctttccaccatccagcagt

SEQ ID NO: 793 - DNA, 21 bp, Artificial Sequence
  Primer
cgagctttacccaccttcagc

SEQ ID NO: 794 - DNA, 20 bp, Artificial Sequence
  Primer
ctggcggtgggctctgtcat

SEQ ID NO: 795 - DNA, 20 bp, Artificial Sequence
  Primer
accgaggcgggagcaagtct

SEQ ID NO: 796 - DNA, 20 bp, Artificial Sequence
  Primer
acgggcggatcgattgtgag

SEQ ID NO: 797 - DNA, 20 bp, Artificial Sequence
  Primer
ggcagcgacatagcgcacct

SEQ ID NO: 798 - DNA, 20 bp, Artificial Sequence
  Primer
agctcaccaccacggctgct

SEQ ID NO: 799 - DNA, 20 bp, Artificial Sequence
  Primer
ctgagacgacggggcgagag

SEQ ID NO: 800 - DNA, 20 bp, Artificial Sequence
  Primer
atcgcgccccttttctgtcc

SEQ ID NO: 801 - DNA, 20 bp, Artificial Sequence
  Primer
gggggcgaccatcaagtgtg

SEQ ID NO: 802 - DNA, 20 bp, Artificial Sequence
  Primer
gacgggccggctgttcttct

SEQ ID NO: 803 - DNA, 20 bp, Artificial Sequence
  Primer
gactccgggcctgggaagag

SEQ ID NO: 804 - DNA, 20 bp, Artificial Sequence
  Primer
actccggccgaaaggtcgag

SEQ ID NO: 805 - DNA, 20 bp, Artificial Sequence
  Primer
ggcggaacacccaccgacta

SEQ ID NO: 806 - DNA, 25 bp, Artificial Sequence
  Primer
ccatgactctggagacatcctggaa

SEQ ID NO: 807 - DNA, 25 bp, Artificial Sequence
  Primer
cgtcagagctctctgttcgctgaag

SEQ ID NO: 808 - DNA, 25 bp, Artificial Sequence
  Primer
ccttctctcgtcttcctcggtcaac

SEQ ID NO: 809 - DNA, 25 bp, Artificial Sequence
  Primer
ccgaacggaccagatggagatagac

SEQ ID NO: 810 - DNA, 25 bp, Artificial Sequence
  Primer
gctcccgagagggataaaacggtaa

SEQ ID NO: 811 - DNA, 25 bp, Artificial Sequence
  Primer
gagtgctctccaaacttggcagttg

SEQ ID NO: 812 - DNA, 25 bp, Artificial Sequence
  Primer
tctcgtcttcctcggtcaacctctt

SEQ ID NO: 813 - DNA, 25 bp, Artificial Sequence
  Primer
ccgaacggaccagatggagatagac

SEQ ID NO: 814 - DNA, 20 bp, Artificial Sequence
  Primer
aacattccgaaggggaccgt

SEQ ID NO: 815 - DNA, 18 bp, Artificial Sequence
  Primer
ggcatccgaaggaggacg

SEQ ID NO: 816 - DNA, 25 bp, Artificial Sequence
  Primer
ggcgctggaaagagggtctactacc

SEQ ID NO: 817 - DNA, 25 bp, Artificial Sequence
  Primer
tgttcaagctgatccctggctatga

SEQ ID NO: 818 - DNA, 25 bp, Artificial Sequence
  Primer
acatctgggactggatatgcgaggt

SEQ ID NO: 819 - DNA, 25 bp, Artificial Sequence
  Primer
atcctcatcgtcccgtttttgacat

SEQ ID NO: 820 - DNA, 25 bp, Artificial Sequence
  Primer
tgtgccaggaccatcttgaattttg

SEQ ID NO: 821 - DNA, 25 bp, Artificial Sequence
  Primer
aggcggatcaaacacttccacatct

SEQ ID NO: 822 - DNA, 25 bp, Artificial Sequence
  Primer
ggggtgcaaatgatacggatgtctt

SEQ ID NO: 823 - DNA, 25 bp, Artificial Sequence
  Primer
agagtatgtggcttccggatgcttg

SEQ ID NO: 824 - DNA, 20 bp, Artificial Sequence
  Primer
acacgccgtgggcctattca

SEQ ID NO: 825 - DNA, 20 bp, Artificial Sequence
  Primer
gccgggaccttggtgctctt

SEQ ID NO: 826 - DNA, 20 bp, Artificial Sequence
  Primer
cacgccgtgggcctattcag

SEQ ID NO: 827 - DNA, 20 bp, Artificial Sequence
  Primer
gccgggaccttggtgctctt

SEQ ID NO: 828 - DNA, 24 bp, Artificial Sequence
  Primer
ctcgcaagcaccctatcaggcagt

SEQ ID NO: 829 - DNA, 24 bp, Artificial Sequence
  Primer
gcagaaagcgtctagccatggcgt

SEQ ID NO: 830 - DNA, 20 bp, Artificial Sequence
  Primer
gcgcctgctgctcgaaatgt

SEQ ID NO: 831 - DNA, 20 bp, Artificial Sequence
  Primer
gtcgcggctgttgcggtagt

SEQ ID NO: 832 - DNA, 20 bp, Artificial Sequence
  Primer
ccccacgtccatctgcgtct

SEQ ID NO: 833 - DNA, 20 bp, Artificial Sequence
  Primer
gcccccagcagtctcaccag

SEQ ID NO: 834 - DNA, 20 bp, Artificial Sequence
  Primer
gctcacgcaccctggaggac

SEQ ID NO: 835 - DNA, 20 bp, Artificial Sequence
  Primer
agttccagcccacgcaccag

SEQ ID NO: 836 - DNA, 25 bp, Artificial Sequence
  Primer
gtgcagtttaggtggcagttcatgc

SEQ ID NO: 837 - DNA, 25 bp, Artificial Sequence
  Primer
ggaaaggggagggtagaaacgtgag

SEQ ID NO: 838 - DNA, 25 bp, Artificial Sequence
  Primer
tgtgattgcgtgtgcagtttaggtg

SEQ ID NO: 839 - DNA, 25 bp, Artificial Sequence
  Primer
ggggagggtagaaacgtgagtctcc

SEQ ID NO: 840 - DNA, 21 bp, Artificial Sequence
  Primer
attccaagcggcctctgataa

SEQ ID NO: 841 - DNA, 21 bp, Artificial Sequence
  Primer
tcttcctctggggcaacttcc

SEQ ID NO: 842 - DNA, 20 bp, Artificial Sequence
  Primer
tcgcagtccccaacctccaa

SEQ ID NO: 843 - DNA, 20 bp, Artificial Sequence
  Primer
cagggtcccgtgctggttgt

SEQ ID NO: 844 - DNA, 20 bp, Artificial Sequence
  Primer
gcagccggtctggagcaaaa

SEQ ID NO: 845 - DNA, 20 bp, Artificial Sequence
  Primer
gcagacggagaaggggacga

SEQ ID NO: 846 - DNA, 20 bp, Artificial Sequence
  Primer
cgcctcattttgcgggtcac

SEQ ID NO: 847 - DNA, 20 bp, Artificial Sequence
  Primer
tggttggcttgtggccagtg

SEQ ID NO: 848 - DNA, 25 bp, Artificial Sequence
  Primer
atcaaggtatgttgcccgtttgtcc

SEQ ID NO: 849 - DNA, 25 bp, Artificial Sequence
  Primer
aggcccactcccataggtattttgc

SEQ ID NO: 850 - DNA, 25 bp, Artificial Sequence
  Primer
cctaggacccctgctcgtgttacag

SEQ ID NO: 851 - DNA, 25 bp, Artificial Sequence
  Primer
gcgataaccaggacaaattggagga

SEQ ID NO: 852 - DNA, 25 bp, Artificial Sequence
  Primer
ctgcgcaccattatcatgcaacttt

SEQ ID NO: 853 - DNA, 25 bp, Artificial Sequence
  Primer
agtagatcccggacggaaggaaaga

SEQ ID NO: 854 - DNA, 20 bp, Artificial Sequence
  Primer
gttcaagcctccaagctgtg

SEQ ID NO: 855 - DNA, 25 bp, Artificial Sequence
  Primer
tcagaaggcaaaaaagagagtaact

SEQ ID NO: 856 - DNA, 20 bp, Artificial Sequence
  Primer
gatgtttgggacgtcacctt

SEQ ID NO: 857 - DNA, 20 bp, Artificial Sequence
  Primer
ctggatgagagccagtcctc

SEQ ID NO: 858 - DNA, 20 bp, Artificial Sequence
  Primer
attgcattggcaaccaaaat

SEQ ID NO: 859 - DNA, 20 bp, Artificial Sequence
  Primer
atctcattgggcatcctgac

SEQ ID NO: 860 - DNA, 20 bp, Artificial Sequence
  Primer
gactggaggttgggaaacaa

SEQ ID NO: 861 - DNA, 20 bp, Artificial Sequence
  Primer
agcagccagagagaatccaa

SEQ ID NO: 862 - DNA, 20 bp, Artificial Sequence
  Primer
taagcatttttcccgcaaag

SEQ ID NO: 863 - DNA, 20 bp, Artificial Sequence
  Primer
aggcattcatgacccatctc

SEQ ID NO: 864 - DNA, 25 bp, Artificial Sequence
  Primer
ccaaccaaatatcattcaggtagac

SEQ ID NO: 865 - DNA, 25 bp, Artificial Sequence
  Primer
gacttcgtgtacctattcactcgat

SEQ ID NO: 866 - DNA, 25 bp, Artificial Sequence
  Primer
gggtttccttatgttcaagaaaaat

SEQ ID NO: 867 - DNA, 25 bp, Artificial Sequence
  Primer
ccaaaactttctctaatggtctcaa

SEQ ID NO: 868 - DNA, 25 bp, Artificial Sequence
  Primer
ttttgctcctctttaccatgctatg

SEQ ID NO: 869 - DNA, 25 bp, Artificial Sequence
  Primer
ggaaatgtctcaggtactttctttg

SEQ ID NO: 870 - DNA, 25 bp, Artificial Sequence
  Primer
aacccaatagcatgacagccaatcc

SEQ ID NO: 871 - DNA, 25 bp, Artificial Sequence
  Primer
tcagccccagagacacggtatatga

SEQ ID NO: 872 - DNA, 25 bp, Artificial Sequence
  Primer
tgaacctgggacctattgatgcaga

SEQ ID NO: 873 - DNA, 25 bp, Artificial Sequence
  Primer
caggggaatctctgccaactttgag

SEQ ID NO: 874 - DNA, 25 bp, Artificial Sequence
  Primer
tgcacagtgacagtgggagaaacac

SEQ ID NO: 875 - DNA, 25 bp, Artificial Sequence
  Primer
aagaatggaaagggttggcagtgtg

SEQ ID NO: 876 - DNA, 25 bp, Artificial Sequence
  Primer
gtgcacagtgacagtgggagaaaca

SEQ ID NO: 877 - DNA, 25 bp, Artificial Sequence
  Primer
aagaatggaaagggttggcagtgtg

SEQ ID NO: 878 - DNA, 17 bp, Artificial Sequence
  Primer
cccacgcgcgcataatg

SEQ ID NO: 879 - DNA, 20 bp, Artificial Sequence
  Primer
ttcacttcggtctcccctag

SEQ ID NO: 880 - DNA, 20 bp, Artificial Sequence
  Primer
tgggccgccaagtacaggaa

SEQ ID NO: 881 - DNA, 20 bp, Artificial Sequence
  Primer
gggttgcccgcctaaaatgg

SEQ ID NO: 882 - DNA, 25 bp, Artificial Sequence
  Primer
ccctattagtggggcagcatgtgtt

SEQ ID NO: 883 - DNA, 25 bp, Artificial Sequence
  Primer
ccaccaagcttttccctgctacatc

SEQ ID NO: 884 - DNA, 25 bp, Artificial Sequence
  Primer
cagtgtcacagccataccaccactg

SEQ ID NO: 885 - DNA, 25 bp, Artificial Sequence
  Primer
tgctgggttcctttattggggaaat

SEQ ID NO: 886 - DNA, 25 bp, Artificial Sequence
  Primer
cccattgcattaatgtaggggcttg

SEQ ID NO: 887 - DNA, 25 bp, Artificial Sequence
  Primer
atcactttcccaccatttgccactt

SEQ ID NO: 888 - DNA, 20 bp, Artificial Sequence
  Primer
cctttccaccatccagcagt

SEQ ID NO: 889 - DNA, 21 bp, Artificial Sequence
  Primer
cgagctttacccaccttcagc

SEQ ID NO: 890 - DNA, 20 bp, Artificial Sequence
  Primer
ggaacaggacctgccgctga

SEQ ID NO: 891 - DNA, 20 bp, Artificial Sequence
  Primer
atcaggtccgccatccgaga

SEQ ID NO: 892 - DNA, 26 bp, Artificial Sequence
  Primer
aaaggtggaagaaaaccagtcccaga

SEQ ID NO: 893 - DNA, 26 bp, Artificial Sequence
  Primer
gccatccgagaatcgtagtgggtatt

SEQ ID NO: 894 - DNA, 20 bp, Artificial Sequence
  Primer
cagcgccagcctgcctctac

SEQ ID NO: 895 - DNA, 20 bp, Artificial Sequence
  Primer
tgctgcactgggcactgctt

SEQ ID NO: 896 - DNA, 25 bp, Artificial Sequence
  Primer
ggaaattaccgactgccctcaaaca

SEQ ID NO: 897 - DNA, 25 bp, Artificial Sequence
  Primer
tgattatttggtccacgctcggttt

SEQ ID NO: 898 - DNA, 20 bp, Artificial Sequence
  Primer
tcccgcgcatccagtagagc

SEQ ID NO: 899 - DNA, 20 bp, Artificial Sequence
  Primer
ctgcggctttgtggcatcct

SEQ ID NO: 900 - DNA, 25 bp, Artificial Sequence
  Primer
tttgctgaaggacaaggtgtgccta

SEQ ID NO: 901 - DNA, 25 bp, Artificial Sequence
  Primer
ccagaagactccgtcaatgttggtg

SEQ ID NO: 902 - DNA, 25 bp, Artificial Sequence
  Primer
aaaaacgtggtcgttccaattctcg

SEQ ID NO: 903 - DNA, 25 bp, Artificial Sequence
  Primer
ccatgcgatagcggctttgtctatt

SEQ ID NO: 904 - DNA, 20 bp, Artificial Sequence
  Primer
tgggaacggtgccaagcatt

SEQ ID NO: 905 - DNA, 21 bp, Artificial Sequence
  Primer
gccacctctgatggacgagca

SEQ ID NO: 906 - DNA, 21 bp, Artificial Sequence
  Primer
cgcgtcaactggggagatgaa

SEQ ID NO: 907 - DNA, 20 bp, Artificial Sequence
  Primer
gcgcgcctgtctgttccaat

SEQ ID NO: 908 - DNA, 25 bp, Artificial Sequence
  Primer
gagtcttctgggttgcaaaggatgg

SEQ ID NO: 909 - DNA, 25 bp, Artificial Sequence
  Primer
cccctggattgagacctgtttcttg

SEQ ID NO: 910 - DNA, 25 bp, Artificial Sequence
  Primer
gcagcattgctctttggtggtaatg

SEQ ID NO: 911 - DNA, 25 bp, Artificial Sequence
  Primer
tgctgaatggtttcacgcttgttct

SEQ ID NO: 912 - DNA, 20 bp, Artificial Sequence
  Primer
ccgcaaacgggtgccattat

SEQ ID NO: 913 - DNA, 20 bp, Artificial Sequence
  Primer
tcgccgtgaggtcctgttcc

SEQ ID NO: 914 - DNA, 20 bp, Artificial Sequence
  Primer
tcgctccaattcccgtggtc

SEQ ID NO: 915 - DNA, 20 bp, Artificial Sequence
  Primer
acgttggcccttcaccatgc

SEQ ID NO: 916 - DNA, 25 bp, Artificial Sequence
  Primer
caagcattacccacaattggctgaa

SEQ ID NO: 917 - DNA, 25 bp, Artificial Sequence
  Primer
ttcttttgccacttctgatggacga

SEQ ID NO: 918 - DNA, 25 bp, Artificial Sequence
  Primer
ttcctttaaaacagccgatggcaac

SEQ ID NO: 919 - DNA, 25 bp, Artificial Sequence
  Primer
tcggaatagcctcatcgctacttgg

SEQ ID NO: 920 - DNA, 20 bp, Artificial Sequence
  Primer
ttccgcctggcacggtactc

SEQ ID NO: 921 - DNA, 20 bp, Artificial Sequence
  Primer
tggcttagcggcatccttgc

SEQ ID NO: 922 - DNA, 20 bp, Artificial Sequence
  Primer
caccatggcctcagccttga

SEQ ID NO: 923 - DNA, 20 bp, Artificial Sequence
  Primer
gtgccgccaacctgccagta

SEQ ID NO: 924 - DNA, 25 bp, Artificial Sequence
  Primer
ggtcttggcactgtggatgatgatt

SEQ ID NO: 925 - DNA, 25 bp, Artificial Sequence
  Primer
gaaaaagggacagctacagcggatg

SEQ ID NO: 926 - DNA, 25 bp, Artificial Sequence
  Primer
cccaatcagaattttggaggctctg

SEQ ID NO: 927 - DNA, 25 bp, Artificial Sequence
  Primer
agcgaattgcacctgaatactgcaa

SEQ ID NO: 928 - DNA, 20 bp, Artificial Sequence
  Primer
tgaccaaaccgagcgtgcag

SEQ ID NO: 929 - DNA, 20 bp, Artificial Sequence
  Primer
cagtggcggggattccattg

SEQ ID NO: 930 - DNA, 20 bp, Artificial Sequence
  Primer
agcgtcaactgctgccacga

SEQ ID NO: 931 - DNA, 20 bp, Artificial Sequence
  Primer
agtaccgtgccaggcggaaa

SEQ ID NO: 932 - DNA, 25 bp, Artificial Sequence
  Primer
aaggtgtgcctattgcaccaggagt

SEQ ID NO: 933 - DNA, 25 bp, Artificial Sequence
  Primer
actagcgacccagaagactccgtca

SEQ ID NO: 934 - DNA, 25 bp, Artificial Sequence
  Primer
agaagaccacttgggctgaccaaac

SEQ ID NO: 935 - DNA, 25 bp, Artificial Sequence
  Primer
ttggcaataggcactccttgtcctt

SEQ ID NO: 936 - DNA, 20 bp, Artificial Sequence
  Primer
gcgccagcctgcctctattg

SEQ ID NO: 937 - DNA, 20 bp, Artificial Sequence
  Primer
tggggcccctctttccaaaa

SEQ ID NO: 938 - DNA, 20 bp, Artificial Sequence
  Primer
atggctcaccgccggtattg

SEQ ID NO: 939 - DNA, 20 bp, Artificial Sequence
  Primer
tgggcgtcactctgcttcca

SEQ ID NO: 940 - DNA, 25 bp, Artificial Sequence
  Primer
gctaaggctgatgaaatggctcacc

SEQ ID NO: 941 - DNA, 25 bp, Artificial Sequence
  Primer
tccaaaaagacaagcatggctgcta

SEQ ID NO: 942 - DNA, 25 bp, Artificial Sequence
  Primer
tctatgttgaaggctcgggaaggtc

SEQ ID NO: 943 - DNA, 25 bp, Artificial Sequence
  Primer
tacttgcttaggctgtccggcatct

SEQ ID NO: 944 - DNA, 20 bp, Artificial Sequence
  Primer
agcagtgcccagtgcagcag

SEQ ID NO: 945 - DNA, 20 bp, Artificial Sequence
  Primer
tgggttcatcaacgccacca